SEQUENCE LISTING (43 sequences)

SEQ ID NO: 1 - protein, 432 aa, Homo sapiens
QVQLVESGGGWQPGRSLRLDCKASGITFSNSGMHWVRQAPGKGLEWVAVRWYDGSKRYYADSVKGRFTISRDNSKNTLFLQMNSLRAEDTAVYYCATNDDYWGQGTLVTVSSASTKGPSVFPLAPCSRSTSESTAALGCLVDYFPEPVTVSWNSGALTSGVHTFPAVLQSSGLYSLSSVVTVPSSSLGTTYTCNVDHKPSNTKVDRVESYGPPCPPCPAPEFLGGPSVFLFPPKPKDTLMISRTPEVTCWVDVSQEDPEVQFNWYYDGVEVHNATKPREEQFNSTYRVVSVLTVLHQDWLNGKEYKCKVSNKGLPSSIEKTISKAGQPREPQVYTLPPSQEEMTKNQVSLTCLVKGFYPSDIAVEWESNGQPEKNYKTTPPVLDSDGSFFLYSRLTVDKSRWQEGNVFSCSVMHEALHNHYTQKSLSLSLGK

SEQ ID NO: 2 - protein, 208 aa, Homo sapiens
EIVLTQSPATLSLSPGERATLSCRASQSVSSYLAWYQQPGQAPRLLIYDASNRATGIPARFSGSGSGTDFTLTISSLEPEDFAVYYCQQSSNWPRTFGQGTKVEIRTVAAPSVFIFPPSDEQLSGTASVVCLLNNFYPREAVQWKVDNALQSGNSQESVTEQDSDSTYSLSSTLTLSKADYEKHKVYACEVTHQGLSSPVTSFNRGEC

SEQ ID NO: 3 - protein, 447 aa, Artificial Sequence
  Pembrolizumab Heavy Chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 218 aa, Artificial Sequence
  Pembrolizumab Light Chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 113 aa, Artificial Sequence
  Humanized Light Chain Variable Domain Sequence
DIVMTQSPDSLAVSLGERATINCKSSQSLWDSGNQKNFLTWYQQKPGQPPKLLIYWTSTRESGVPDRFSGSGSGTDFTLTISSLQAEDVAVYYCQNDYFYPLTFGGGTKVEIK

SEQ ID NO: 6 - protein, 117 aa, Artificial Sequence
  Humanized Heavy Chain Variable Domain Sequence
QVQLVQSGAEVKKPGASVKVSCKASGYTFTSYWINWVRQAPGQGLEQMGNIYPGSSLTNYNEKFKNRVTMTRDTSTSTVYMELSSLRSEDTAVYYCARLLTGTFAYWGQGTLVTVSS

SEQ ID NO: 7 - protein, 113 aa, Artificial Sequence
  Humanized Light Chain Variable Domain Sequence
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPLTFGGGTKVEIK

SEQ ID NO: 8 - protein, 117 aa, Artificial Sequence
  Humanized Heavy Chain Variable Domain
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLLTGTFAYWGQGTLVTVSS

SEQ ID NO: 9 - protein, 113 aa, Artificial Sequence
  Humanized Light Chain Variable Domain
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIK

SEQ ID NO: 10 - protein, 117 aa, Artificial Sequence; identical to SEQ ID NO: 8
  Humanized Heavy Chain Variable Domain

SEQ ID NO: 11 - protein, 113 aa, Artificial Sequence
  Human Light Chain Variable Domain
DIVMTQSPDSLAVSLGERATINCKSSQSLWDSTNQKNFLTWYQQKPGQPPKLLIYWTSTRESGVPDRFSGSGSGTDFTLTISSLQAEDVAVYYCQNDYFYPLTFGGGTKVEIK

SEQ ID NO: 12 - protein, 117 aa, Artificial Sequence; identical to SEQ ID NO: 8
  Human Heavy Chain Variable Domain

SEQ ID NO: 13 - protein, 112 aa, Artificial Sequence
  Humanized Light Chain Variable Domain
DIVTQSPDSLAVSLGERATINCKSSQSLWDSGNQKNFLTWYQQKPGQPPKLLIYWTSTRESGVPDRFSGSGSGTDFTLTISSLQAEDVAVYYCQNDYFYPLTFGGGTKVEIK

SEQ ID NO: 14 - protein, 117 aa, Artificial Sequence
  Humanized Heavy Chain Variable Domain
QVQLVWSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSS

SEQ ID NO: 15 - protein, 113 aa, Artificial Sequence; identical to SEQ ID NO: 7
  Humanized Light Chain Variable Domain

SEQ ID NO: 16 - protein, 117 aa, Artificial Sequence
  Humanized Heavy Chain Variable Domain
QVQLVQSGAEVKKPGASVKVSCKASGYTFTSYWINWVRQAPGQGLEQMGNIYPGSSLTNYNEKFKNRVTMTRDTSTSTVYMELSSLRSEDTAVYYCARLSTGTFAYWGQGTLCTVSS

SEQ ID NO: 17 - protein, 444 aa, Artificial Sequence
  Humanized Heavy Chain
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 443 aa, Artificial Sequence
  Humanized Heavy Chain
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 19 - protein, 221 aa, Artificial Sequence
  Humanized Light Chain
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 117 aa, Artificial Sequence
  Humanized Light Chain Variable Domain
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSS

SEQ ID NO: 21 - protein, 117 aa, Artificial Sequence
  Humanized Heavy Chain Variable Domain
QVQLVQSGAEVKKPGASVKVSCKASGYTFTSYWINWVRQAPGQGLEWMGNIWPGSSLTNYNEKFKNRVTMTRDTSTSTVYMELSSLRSEDTAVYYCARLLTGTFAYWGQGTLVTVSS

SEQ ID NO: 22 - protein, 112 aa, Artificial Sequence
  Humanized Light Chain Variable Domain
DIVTQSPDSLAVSLGERATINCKSSQSLWDSTNQKNFLTWYQQKPGQPPKLLIYWTSTRESGVPDRFSGSGSGTDFTLTISSLQAEDVAVYYCQNDYFYPLTFGGGTKVEIK

SEQ ID NO: 23 - protein, 116 aa, Artificial Sequence
  Humanized Heavy Chain Variable Domain
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQTLVTVSS

SEQ ID NO: 24 - protein, 113 aa, Artificial Sequence; identical to SEQ ID NO: 5
  Light Chain Variable Domain

SEQ ID NO: 25 - protein, 117 aa, Artificial Sequence
  Humanized Heavy Chain Variable Domain
QVQLVQSGAEVKKPGASVKVSCKASGYTFTSYWINWVRQAPGQGLEWMGNIYPGSSITNYNEKFKNRVTMTRDTSTSTVYMELSSLRSEDTAVYYCARLTTGTFAYWGQGTLVTVSS

SEQ ID NO: 26 - protein, 113 aa, Artificial Sequence; identical to SEQ ID NO: 7
  Humanized Light Chain Variable Domain

SEQ ID NO: 27 - protein, 117 aa, Artificial Sequence; identical to SEQ ID NO: 25
  Humanized Heavy Chain Variable Domain

SEQ ID NO: 28 - protein, 113 aa, Artificial Sequence; identical to SEQ ID NO: 9
  Humanized Light Chain Variable Domain

SEQ ID NO: 29 - protein, 117 aa, Artificial Sequence; identical to SEQ ID NO: 25
  Humanized Heavy Chain Variable Domain

SEQ ID NO: 30 - protein, 113 aa, Artificial Sequence; identical to SEQ ID NO: 11
  Humanized Light Chain Variable Domain

SEQ ID NO: 31 - protein, 117 aa, Artificial Sequence; identical to SEQ ID NO: 25
  Humanized Heavy Chain Variable Domain

SEQ ID NO: 32 - protein, 113 aa, Artificial Sequence; identical to SEQ ID NO: 5
  Humanized Light Chain Variable Domain

SEQ ID NO: 33 - protein, 117 aa, Artificial Sequence; identical to SEQ ID NO: 21
  Humanized Heavy Chain Variable Domain

SEQ ID NO: 34 - protein, 113 aa, Artificial Sequence; identical to SEQ ID NO: 7
  Humanized Light Chain Variable Domain

SEQ ID NO: 35 - protein, 117 aa, Artificial Sequence; identical to SEQ ID NO: 21
  Humanized Heavy Chain Variable Domain

SEQ ID NO: 36 - protein, 113 aa, Artificial Sequence; identical to SEQ ID NO: 9
  Humanized Light Chain Variable Domain

SEQ ID NO: 37 - protein, 113 aa, Artificial Sequence; identical to SEQ ID NO: 11
  Humanized Light Chain Variable Domain

SEQ ID NO: 38 - protein, 117 aa, Artificial Sequence; identical to SEQ ID NO: 21
  Humanized Heavy Chain Variable Domain

SEQ ID NO: 39 - protein, 432 aa, Artificial Sequence
  Anti-PD-1 fusion protein AMP-224
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWEPKSCDKTHTCPPCPAPELLGGPSVFLFPPKPKDTLMISRTPEVTCWVDVSHEDPEVKFNWYVDGVEVHNAKTKPREEQYNSTYRWSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - DNA, 25 bp, Artificial Sequence
  PD-1 Binding Antagonist Aptamer
gctactgtacatcacgcctctcccc

SEQ ID NO: 41 - DNA, 24 bp, Artificial Sequence
  PD-1 Binding Antagonist Aptamer
ctactgtacatcacgcctctcccc

SEQ ID NO: 42 - DNA, 25 bp, Artificial Sequence
  PD-1 Binding Antagonist Aptamer
gtacagttcccgtccctgcactaca

SEQ ID NO: 43 - DNA, 24 bp, Artificial Sequence
  PD-1 Binding Antagonist Aptamer
gtacagttcccgtcctgcactaca

(19) United States
(12) Patent Application Publication
(10) Pub. No.: US 2019/0211102 A1
(43) Pub. Date: Jul. 11, 2019

(54) METHODS AND COMBINATION THERAPY TO TREAT CANCER

(71) Applicant: Array BioPharma Inc., Boulder, CO (US)

(72) Inventors: Patrice A. Lee, Boulder, CO (US); David Chantry, Boulder, CO (US); Shannon L. Winski, Boulder, CO (US)

(21) Appl. No.: 16/244,827

(22) Filed: Jan. 10, 2019

Related U.S. Application Data
(60) Provisional application No. 62/615,911, filed on Jan. 10, 2018.

(51) Int. Cl. C07K 16/28; A61P 35/04; A61K 9/00; A61K 31/4439

(52) U.S. Cl. CPC C07K 16/2818; A61P 35/04; A61K 31/4439; A61K 9/0053; A61K 9/0019

(57) ABSTRACT

This invention relates to a method of treating cancer by administering a combination therapy comprising a combination of CSF-1R inhibitor, which is ARRY-382 or a pharmaceutically acceptable salt thereof, and a PD-1 binding antagonist, to a patient in need thereof.

### CROSS-REFERENCE TO RELATED APPLICATIONS

This application claims priority to U.S. Application Ser. No. 62/615,911, filed on Jan. 10, 2018, the entire contents of which are hereby incorporated by reference.

### FIELD

The present invention relates to methods and combination therapies useful for the treatment of cancer. In particular, this invention relates to methods and combination therapies for treating cancer by administering a combination therapy comprising a CSF-1R inhibitor which is ARRY-382 or a pharmaceutically acceptable salt thereof, and PD-1 binding antagonist. Pharmaceutical uses of the combination of the present invention are also described.

### BACKGROUND

Macrophages are among the immune cells that infiltrate solid tumors. In many cancers, higher levels of macrophage infiltration have been associated with poorer prognosis; head and neck, skin, melanoma, mesothelioma, breast, ovarian, uterine, cervical, bladder, kidney, pancreatic, liver, thyroid and brain are among the primary cancers that have shown to have a clinical outcome adversely affected by the presence of macrophages. Ruffell & Coussens, Cancer Cell (2015) 27 (4):462.

Tumor-associated macrophages (TAM) are abundant in tumors, and promote growth, angiogenesis, and metastasis through secretion of proangiogenic factors and remodeling of the tumor stroma. In addition, TAMs are known to cause suppression of anticancer immune responses through direct inhibition of anti-tumor T cells by production of reactive oxygen species and suppressive cytokines. Id. Therefore, TAMs have been identified as a potential target for cancer therapeutics.

CSF-1R, also known as M-CSFR or CD-I 15, is a tyrosine kinase receptor expressed selectively on macrophage and granulocyte cell lineages in normal individuals and on tumor cells in cancer. Upon ligand binding, CSF-1R dimerizes, leading to trans-phosphorylation of the receptor and phosphorylation, and activation of downstream signaling molecules, such as MAPK and Akt. Phosphorylation of CSF-1R results in: (1) the proliferation and differentiation of macrophages from hematopoietic progenitor stem cells, (2) survival and migration of macrophages to various organs and tissues in the body, particularly the tumor stroma, and (3) maintenance of the immune suppressive phenotype of TAM and other CSF-1R expressing cells of the myeloid lineage. Given the critical role of CSF-1R in the regulation and survival of TAMs and other myeloid cells, CSF-1R inhibition has been identified as a potential cancer target.

Programmed cell death 1 (PD-1) and PD-L1 are part of an immune checkpoint pathway used normally in maintenance of self-tolerance and control of T cell activation, but cancer cells can use the pathway to suppress the anti-tumor response and prevent their destruction. Clinical research has found that targeting the PD-1/PD-L1 axis with antagonist antibodies to either protein can cause tumor regression in patients and prolong survival. However, many patients still do not receive a benefit. CSF-1R expression may be one factor that impacts response. CSF-1R expressing cells of the myeloid linage, including TAM and myeloid derived suppressor cells (MDSCs) have been implicated in potentially contributing to the immune suppression that prevent the effectiveness of PD-1/PD-LI targeted therapies.

Unfortunately, despite more therapeutic options for solid tumors using immunotherapy, treatment options for indications where macrophages play a major role in immune suppression still remain elusive. There exists a need for more and different therapies that can effectively eliminate the influence of TAM in the tumor microenvironment and relieve the suppression they exert. TAMs and MDSC also appear to be an adaptive mechanism of suppression that is activated during immunotherapy and limits its usefulness. Targeting these cells could increase the number of patients that respond to immunotherapy and prevent recurrence. Targeting CSF-1R on TAMs and MDSCs could relieve immune suppression of anti-tumor T cells, reducing their exhaustion in the tumor and enhancing their activity.

There remains a need for such an advantageous therapy for treating, stabilizing, preventing, and/or delaying development of various cancers.

### SUMMARY

In one embodiment, provided herein is a combination therapy method comprising administering, over a period of time, to a patient in need thereof, therapeutically effective amounts, independently, of a CSF-1R inhibitor which is ARRY-382 or a pharmaceutically acceptable salt thereof, and a PD-1 binding antagonist.

In one embodiment, provided herein is a combination therapy method comprising administering to a patient in need thereof, therapeutically effective amounts, independently, of a CSF-1R inhibitor which is ARRY-382 or a pharmaceutically acceptable salt thereof, and a PD-1 binding antagonist.

Also provided herein are combination therapy methods that consist essentially of administering, over a period of time, to a patient in need thereof, therapeutically effective amounts, independently, of a CSF-1R inhibitor which is ARRY-382 or a pharmaceutically acceptable salt thereof, and a PD-1 binding antagonist.

Also provided herein are combination therapy methods that consist essentially of administering to a patient in need thereof, therapeutically effective amounts, independently, of a CSF-1R inhibitor which is ARRY-382 or a pharmaceutically acceptable salt thereof, and a PD-1 binding antagonist.

In one embodiment, the invention provides a method for treating cancer comprising administering to a patient in need thereof, over a period of time, a combination therapy comprising an amount of a PD-1 binding antagonist and an amount of a CSF-1R inhibitor which is ARRY-382 or a pharmaceutically acceptable salt thereof, wherein the amounts together are effective in treating cancer.

In one embodiment, the invention provides a method for treating cancer comprising administering to a patient in need thereof, a combination therapy comprising an amount of a PD-1 binding antagonist and an amount of a CSF-1R inhibitor which is ARRY-382 or a pharmaceutically acceptable salt thereof, wherein the amounts together are effective in treating cancer.

Also provided are methods for treating cancer that consist essentially of administering to a patient need thereof, over a period of time, a combination therapy comprising, consisting essentially of, or consisting of an amount of a PD-1 binding antagonist and an amount of a CSF-1R inhibitor which is ARRY-382 or a pharmaceutically acceptable salt thereof, wherein the amounts together are effective in treating cancer.

Also provided are methods for treating cancer that consist essentially of administering to a patient need thereof, a combination therapy comprising, consisting essentially of, or consisting of an amount of a PD-1 binding antagonist and an amount of a CSF-1R inhibitor which is ARRY-382 or a pharmaceutically acceptable salt thereof, wherein the amounts together are effective in treating cancer.

In one embodiment, the PD-1 binding antagonist is an anti PD-1 antibody. In one embodiment, the PD-1 binding antagonist is an anti PD-1 antibody selected from nivolumab, pembrolizumab, a biosimilar of nivolumab, or a biosimilar of pembrolizumab.

In another embodiment, the invention provides a method for treating cancer comprising or consisting essentially of administering to a patient in need thereof, during a period of time, a combination therapy comprising, consisting essentially of, or consisting of an amount of a PD-1 binding antagonist which is pembrolizumab or a biosimilar of pembrolizumab, and an amount of a CSF-1R inhibitor which is ARRY-382 or a pharmaceutically acceptable salt thereof, wherein the amounts together are effective in treating cancer.

In another embodiment, the invention provides a method for treating cancer comprising or consisting essentially of administering to a patient in need thereof, a combination therapy comprising, consisting essentially of, or consisting of an amount of a PD-1 binding antagonist which is pembrolizumab or a biosimilar of pembrolizumab, and an amount of a CSF-1R inhibitor which is ARRY-382 or a pharmaceutically acceptable salt thereof, wherein the amounts together are effective in treating cancer.

In another embodiment, the invention provides a method for treating cancer comprising or consisting essentially of administering to a patient in need thereof, during a period of time, a combination therapy comprising, consisting essentially of, or consisting of an amount of a PD-1 binding antagonist which is nivolumab or a biosimilar thereof, and an amount of a CSF-1R inhibitor which is ARRY-382 or a pharmaceutically acceptable salt thereof, wherein the amounts together are effective in treating cancer.

In another embodiment, the invention provides a method for treating cancer comprising or consisting essentially of administering to a patient in need thereof, a combination therapy comprising, consisting essentially of, or consisting of an amount of a PD-1 binding antagonist which is nivolumab or a biosimilar thereof, and an amount of a CSF-1R inhibitor which is ARRY-382 or a pharmaceutically acceptable salt thereof, wherein the amounts together are effective in treating cancer.

In some embodiments of any of the methods described herein, before the period of time, the patient was treated with one or more therapeutic agent(s) that did not comprise, consist essentially of, or consist of a PD-1 binding antagonist and an amount of ARRY-382 or a pharmaceutically acceptable salt thereof (e.g., therapeutic agents that consist of a PD-1 binding antagonist and an amount of a ARRY-382 or a pharmaceutically acceptable salt thereof). In some embodiments of any of the methods described herein, before the period of time, the patient has been treated with a platinum-based chemotherapeutic agent, and optionally, the patient has been previously determined to be non-responsive to treatment with the platinum-based chemotherapeutic agent. In some embodiments of any of the methods described herein, before the period of time, the patient has been treated with a BRAF kinase inhibitor (e.g., encorafenib), and optionally, the prior treatment with the BRAF kinase inhibitor (e.g., encorafenib) was unsuccessful. In some embodiments of any of the methods described herein, before the period of time, the patient was treated with a CSF-1R inhibitor (e.g., ARRY-382) as a monotherapy, and, optionally, the prior treatment with the CSF-1R inhibitor as a monotherapy was unsuccessful. In some embodiments of any of the methods described herein, before the period of time, the patient was treated with a PD-1 binding antagonist (e.g., nivolumab or pembrolizumab, or a biosimilar thereof) as a monotherapy, and optionally, the prior treatment with the PD-1 binding antagonist (e.g., nivolumab or pembrolizumab, or a biosimilar thereof) was unsuccessful. In some embodiments of any of the methods described herein, before the period of time, the patient was treated with one or more of a chemotherapy, a targeted anticancer agent, radiation therapy, and surgery, and optionally, the prior treatment was unsuccessful. In some embodiments of any of the methods described herein, before the period of time, the patient was treated with one or both of a platinum-based chemotherapy and a fluoropyrimidine-containing therapy or therapeutic agent. In some embodiments of any of the methods described herein, before the period of time, the patient was treated with one or both of a EGFR inhibitor and an ALK inhibitor, and optionally, the prior treatment was unsuccessful. In some embodiments of any of the methods described herein, before the period of time, the patient was treated with one or more of folinic acid, fluorouracil, oxaliplatin, and irinotecan, and optionally, the prior treatment was unsuccessful. In some embodiments of any of the methods described herein, before the period of time, the patient was treated with one or more therapeutic agents selected from the group of paclitaxel, gemcitabine, carboplatin, cisplatin, and doxorubicin, and optionally, the prior treatment was unsuccessful.

In some embodiments of any of the methods described herein, after the period of time, the patient is treated with therapeutic agents that did not comprise, consist essentially of, or consist of a PD-1 binding antagonist and ARRY-382 or a pharmaceutically acceptable salt thereof.

In some embodiments, administration of the PD-1 binding antagonist and administration of ARRY-382 or a pharmaceutically acceptable salt thereof during the period of time, occurs at substantially the same time. In some embodiments, administration of the PD-1 binding antagonist to the patient occurs prior to administration of ARRY-382 or a pharmaceutically acceptable salt thereof to the patient, during the period of time. In some embodiments, administration of ARRY-382 or a pharmaceutically acceptable salt thereof to the patient occurs prior to administration of the PD-1 binding antagonist to the patient, during the period of time.

In some embodiments, the patient is also administered surgical treatment (e.g., resection of a solid tumor and/or lymph node) and/or chemotherapy during the period of time. In some embodiments, the patient is also administered a targeted anticancer agent during the period of time. In some embodiments, the patient is also administered radiation therapy during the period of time. In some embodiments, a patient is administered one or more agents to ameliorate side effects of treatment during the period of time (e.g., one or more of corticosteroids, serotonin antagonists, dopamine antagonists, NK-1 inhibitors, cannabinoids, anti-anxiety drugs (e.g., lorazepam or diazepam), antibiotics, anti-fungal agents, colony-stimulating factor, iron supplements, Procrit, epoetin alfa, darbepoetin alfa, anti-emetics, diuretics, NSAIDs, analgesics, methotrexate, anti-diuretics, probiotics, blood pressure medications, anti-nausea agents, laxatives, etc.).

In some embodiments of any of the methods described herein, the patient is not administered a BRAF kinase inhibitor (e.g., encorafenib) during the period of time. In some embodiments of any of the methods described herein, the patient is not administered an additional targeted anticancer agent during the period of time. In some embodiments of any of the methods described herein, the subject is not administered chemotherapy during the period of time. In some embodiments of any of the methods described herein, the subject is not administered a kinase inhibitor during the period of time. In some embodiments of any of the methods described herein, the patient is not administered one or more of alkylating agents, anthracyclines, cytoskeletal disruptors (e.g., taxanes), epothilones, histone deacetylase inhibitors, topoisomerase I inhibitors, topoisomerase II inhibitors, nucleotide analogs, nucleotide precursor analogs, peptide antibiotics, platinum-based agents, retinoids, and vinca alkaloids and derivatives thereof, during the period of time. In some embodiments of any of the methods described herein, the patient is not administered a c-MET inhibitor during the period of time. In some embodiments of any of the methods described herein, the subject is not administered a CDK4/6 inhibitor during the period of time. In some embodiments of any of the methods described herein, the patient is not administered a PI3K inhibitor during the period of time. In some embodiments of any of the methods described herein, the subject is not administered a BRAF inhibitor (e.g., encorafenib) during the period of time. In some embodiments of any of the methods described herein, the patient is not administered a FGFR inhibitor during the period of time. In some embodiments of any of the methods described herein, the subject is not administered a MEK inhibitor during the period of time. In some embodiments of any of the methods described herein, the patient is not administered a BCR-ABL inhibitor during the period of time. In some embodiments of any of the methods described herein, the patient is not administered a different CSR-1R inhibitor (e.g., pexidartinib (PLX3397)) than the one administered during the period of time.

In some embodiments, “consisting essentially of,” during the period of time, can include a surgical treatment and/or chemotherapy. In some embodiments, “consisting essential of,” during the period of time, can include one or more types of chemotherapeutic agents selected from the group of: alkylating agents, anthracyclines, cytoskeletal disruptors (e.g., taxanes), epothilones, histone deacetylase inhibitors, topoisomerase I inhibitors, topoisomerase II inhibitors, kinase inhibitors with the exclusion of a BRAF kinase inhibitor), nucleotide analogs, nucleotide precursor analogs, peptide antibiotics, platinum-based agents, retinoids, and vinca alkaloids and derivatives. In some embodiments, “consisting essentially of” during the period of time, can include treatment with any targeted chemotherapeutic agent, except for one or more of the following: a c-MET inhibitor, a CDK4/6 inhibitor, a PI3K inhibitor, a BRAF inhibitor, a FGFR inhibitor, a MEK inhibitor, and a BCR-ABL inhibitor. In some embodiments, “consisting essentially of” during the period of time can include radiation therapy.

### BRIEF DESCRIPTION OF DRAWINGS

FIG. 1A is a schematic representation of an immunosuppressive tumor microenvironment showing an interaction between a colony-stimulating-factor-1 (CSF-1)-induced tumor-associated macrophage (TAM), an effector T cell and a tumor cell. PD-1=programmed cell death protein 1 (PD-1); PD-L1=programmed death ligand 1

FIG. 1B is a schematic representation of a putative mechanism of action of combined inhibition of CSF-1 receptor (CSF-1R) and programmed cell death protein 1 (PD-1) (Zhu et al. Cancer Res. 2014; 74(18):5057-5069). Array-382=a CSF-1R inhibitor; pembrolizumab=a PD-1 inhibitor

FIG. 2 is a flow chart of a study design in three parts: Part A, Part B and Part C. Part A is a dose escalation study in which patients received a combined therapy of pembrolizumab and ARRY-382 (200 mg, 400 mg or 300 mg). Part B is a melanoma biomarker cohort study to explore the effect on circulating biomarkers of immune response and bone turnover. Part C is a non-small cell lung cancer (NSCLC) study to estimate the efficacy of pembrolizumab and ARRY-382, based on overall response rate (ORR).

FIG. 3 is a graph showing the geometric mean plasma concentration of ARRY-384 (ng/mL) over time. AUC₀₋₂₄=area under the plasma concentration-time curve over the dosing interval of 0-24 hours; GMean=geometric mean; IC₅₀=half-maximal inhibitory concentration; QD=once daily FIG. 4 is a bar graph showing the mean maximum-fold change from baseline serum in CSF-1 levels for ARRY-382 200 mg+pembrolizumab, ARRY-382 300 mg+pembrolizumab; and ARRY-382 400 mg+pembrolizumab.

FIG. 5 is a bar graph of the percentage of change from baseline in patients enrolled in the Part A study.

### DETAILED DESCRIPTION

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

### General Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

“About” when used to modify a numerically defined parameter (e.g., the dose of a CSF-1R inhibitor or a PD-1 binding antagonist, or the length of treatment time with a combination therapy described herein) means that the parameter may vary by as much as 10% below or above the stated numerical value for that parameter. For example, a dose of about 5 mg/kg may vary between 4.5 mg/kg and 5.5 mg/kg. “About” when used at the beginning of a listing of parameters is meant to modify each parameter. For example, about 0.5 mg, 0.75 mg or 1.0 mg means about 0.5 mg, about 0.75 mg or about 1.0 mg. Likewise, about 5% or more, 10% or more, 15% or more, 20% or more, and 25% or more means about 5% or more, about 10% or more, about 15% or more, about 20% or more, and about 25% or more.

The phrases “prior to a period of time” or “before a period of time” refer to (1) the completion of administration of surgery and/or radiation treatment to the subject before the first administration of a therapeutic agent during the period of time, and/or (2) the administration of one or more therapeutic agents to the subject before a first administration of a therapeutic agent in the combination therapy during the period of time, such that the one or more therapeutic agents are present in subtherapeutic and/or undetectable levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in subtherapeutic levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in undetectable levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in subtherapeutic and/or undetectable levels in the subject at the time the first administration of a therapeutic agent in the combination therapy is performed during the period of time.

The phrase “pharmaceutically acceptable” indicates that the substance or composition must be compatible chemically and/or toxicologically, with the other ingredients comprising a formulation, and/or the mammal being treated therewith. Some embodiments relate to the pharmaceutically acceptable salts of the compounds described herein. The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined.

“Administration”, “administering”, “treating”, and “treatment,” as it applies to a patient, individual, animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell.

“Treatment” and “treating”, as used in a clinical setting, is intended for obtaining beneficial or desired clinical results. For purposes of this invention, beneficial or desired clinical results include, but are not limited to, one or more of the following: reducing the proliferation of (or destroying) neoplastic or cancerous cells, inhibiting metastasis of neoplastic cells, shrinking or decreasing the size of a tumor, remission of a disease (e.g., cancer), decreasing symptoms resulting from a disease (e.g., cancer), increasing the quality of life of those suffering from a disease (e.g., cancer) (e.g., assessed using FACT-G or EORTC-QLQC30), decreasing the dose of other medications required to treat a disease (e.g., cancer), delaying the progression of a disease (e.g., cancer), and/or prolonging survival of patients having a disease (e.g., cancer). For example, treatment can be the diminishment of one or several symptoms of a disorder, such as cancer. Within the meaning of the present invention, the term “treat” also denotes to arrest, delay the onset (i.e., the period prior to clinical manifestation of a disease) and/or reduce the risk of developing or worsening a disease. “Treatment” can also mean prolonging survival as compared to expected survival if not receiving treatment, for example, an increase in overall survival (OS) compared to a subject not receiving treatment as described herein, and/or an increase in progression-free survival (PFS) compared to a subject not receiving treatment as described herein. The term “treating” can also mean an improvement in the condition of a subject having a cancer, e.g., one or more of a decrease in the size of one or more tumor(s) in a subject, a decrease or no substantial change in the growth rate of one or more tumor(s) in a subject, a decrease in metastasis in a subject, and an increase in the period of remission for a subject (e.g., as compared to the one or more metric(s) in a subject having a similar cancer receiving no treatment or a different treatment, or as compared to the one or more metric(s) in the same subject prior to treatment). Additional metrics for assessing response to a treatment in a subject having a cancer are disclosed herein below.

The term “subject” includes any organism, preferably an animal, more preferably a mammal (e.g., rat, mouse, dog, cat, and rabbit) and most preferably a human.

A “patient” to be treated according to this invention includes any warm-blooded animal, such as, but not limited to human, monkey or other lower-order primate, horse, dog, rabbit, guinea pig, or mouse. In one embodiment the patient is human. In one embodiment, the patient is a pediatric patient. Those skilled in the medical art are readily able to identify individuals who are afflicted with cancer and who are in need of treatment.

The term “pediatric patient” as used herein refers to a patient under the age of 16 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994.

The terms “treatment regimen” and “dosing regimen” are used interchangeably to refer to the dose and timing of administration of each therapeutic agent in a combination of the invention.

The term “regulatory agency” is a country's agency for the approval of the medical use of pharmaceutical agents with the country. For example, a non-limiting example of a regulatory agency is the U.S. Food and Drug Administration (FDA).

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also antigen binding fragments thereof (such as Fab, Fab′, F (ab′)₂, Fv), single chain (scFv) and domain antibodies (including, for example, shark and camelid antibodies), and fusion proteins comprising an antibody, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding fragment” or “antigen binding portion” of an antibody, as used herein, refers to one or more fragments of an intact antibody that retain the ability to specifically bind to a given antigen (e.g., PD-1). Antigen binding functions of an antibody can be performed by fragments of an intact antibody. Examples of binding fragments encompassed within the term “antigen binding fragment” of an antibody include Fab; Fab′; F(ab′)₂; an Fd fragment consisting of the VH and CH1 domains; an Fv fragment consisting of the VL and VH domains of a single arm of an antibody; a single domain antibody (dAb) fragment (Ward et al., Nature 341:544-546, 1989), and an isolated complementarity determining region (CDR).

An antibody, an antibody conjugate, or a polypeptide that “preferentially binds” or “specifically binds” (used interchangeably herein) to a target (e.g., PD-1 protein) is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a PD-1 epitope is an antibody that binds this epitope with greater affinity, avidity, more readily, and/or with greater duration than it binds to other PD-1 epitopes or non-PD-1 epitopes. It is also understood that by reading this definition, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al., 1997, J. Molec. Biol. 273:927-948). As used herein, a CDR may refer to CDRs defined by either approach or by a combination of both approaches.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs may be identified as the residues that make enthalpic contributions to antigen binding. See, e.g., Makabe et al., Journal of Biological Chemistry, 283:1156-1166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original | |
|---|---|
| residue | Conservative substitution |
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

The term “PD-1 binding antagonist” as used herein refers to a molecule that binds specifically to PD-1 and decreases the interaction of PD-1 with one or more of its binding partners, such as PD-L1 and/or PD-L2. For example, PD-1 binding antagonists include anti-PD-1 antibodies, antigen binding fragments thereof, immunoadhesins, aptamers, fusion proteins, and oligopeptides. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional. In some embodiments, the PD-1 binding antagonist is an anti-PD-1 antibody. In one embodiment, the PD-1 binding antagonist is an anti-PD-1 antibody selected from nivolumab, a biosimilar of nivolumab, pembrolizumab, and a biosimilar of pembrolizumab. In one embodiment, the PD-1 binding antagonist is nivolumab or a biosimilar thereof. In one embodiment, the PD-1 binding antagonist is pembrolizumab or a biosimilar thereof.

An anti-PD-1 antibody as described herein can also be an antigen-binding antibody fragment of nivolumab or a biosimilar thereof, or an antigen-binding antibody fragment of pembrolizumab or a biosimilar thereof.

A “biosimilar” means an antibody or antigen-binding fragment that has the same primary amino acid sequence as compared to a reference antibody (e.g., nivolumab or pembrolizumab) and optionally, may have detectable differences in post-translation modifications (e.g., glycosylation and/or phosphorylation) as compared to the reference antibody (e.g., a different glycoform).

In some examples, an anti-PD-1 antibody can be a biosimilar of nivolumab, or a biosimilar of pembrolizumab. In some embodiments, a biosimilar is an antibody or antigen-binding fragment thereof that has a light chain that has the same primary amino acid sequence as compared to a reference antibody (e.g., nivolumab or pembrolizumab) and a heavy chain that has the same primary amino acid sequence as compared to the reference antibody. In some examples, a biosimilar is an antibody or antigen-binding fragment thereof that has a light chain that includes the same light chain variable domain sequence as a reference antibody (e.g., nivolumab or pembrolizumab) and a heavy chain that includes the same heavy chain variable domain sequence as a reference antibody. In some embodiments, a biosimilar can have a similar glycosylation pattern as compared to the reference antibody (e.g., nivolumab or pembrolizumab). In other embodiments, a biosimilar can have a different glycosylation pattern as compared to the reference antibody (e.g., nivolumab or pembrolizumab).

Table 2 below provides a list of the amino acid sequences of exemplary PD-1 binding antagonists for use in the treatment method, medicaments and uses of the present invention. CDRs are underlined for mAb7 and mAb15. The mAB7 is also known as RN888 or PF-6801591. mAb7 (aka RN888) and mAb15 are disclosed in International Patent Publication No. WO2016/092419, the disclosure of which is hereby incorporated by reference in its entirety.

TABLE 2

| | |
|---|---|
| Nivolumab, MDX1106, | QVQLVESGGGWQPGRSLRLDCKASGITFSNSGMHWVRQ |
| full length heavy chain | APGKGLEWVAVrWYDGSKRYYADSVKGRFTISRDNSKNT |
| From WO 2006/121168 | LFLQMNSLRAEDTAVYYCATNDDYWGQGTLVTVSSASTK |
| | GPSVFPLAPCSRSTSESTAALGCLVDYFPEPVTVSWNSG |
| | ALTSGVHTFPAVLQSSGLYSLSSVVTVPSSSLGTTYTCNV |
| | DHKPSNTKVDRVESYGPPCPPCPAPEFLGGPSVFLFPPK |
| | PKDTLMISRTPEVTCWVDVSQEDPEVQFNWYYDGVEVHN |
| | ATKPREEQFNSTYRWSVLTVLHQDWLNGKEYKCKVSNK |
| | GLPSSIEKTISKA |
| | GQPREPQVYTLPPSQEEMTKNQVSLTCLVKGFYPSDIAVE |
| | WESNGQPEKNYKTTPPVLDSDGSFFLYSRLTVDKSRWQE |
| | GNVFSCSVMHEALHNHYTQKSLSLSLGK (SEQ ID NO: 1) |
| Nivolumab, MDX1106, | EIVLTQSPATLSLSPGERATLSCRASQSVSSYLAWYQQPG |
| full length light chain | QAPRLLIYDASNRATGIPARFSGSGSGTDFTLTISSLEPED |
| From WO 2006/121168 | FAVYYCQQSSNWPRTFGQGTKVEIRTVAAPSVFIFPPSDE |
| | QLSGTASVVCLLNNFYPREAVQWKVDNALQSGNSQESVT |
| | EQDSDSTYSLSSTLTLSKADYEKHKVYACEVTHQGLSSPV |
| | TSFNRGEC (SEQ ID NO: 2) |
| Pembrolizumab, | QVQLVQSGVEVKKPGASVKVSCKASGYTFTNYYMYWV |
| MK3475, full length | RQAPGQGLEWMGGINPSNGGTNFNEKFKNRVTLTTDS |
| heavy chain | STTTAYMELKSLQFDDTAVYYCARRDYRFDMGFDYWG |
| From WO 2009/114335 | QGTTVTVSSASTKGPSVFPLAPCSRSTSESTAALGCLVK |
| | DYFPEPVTVSWNSGALTSGVHTFPAVLQSSGLYSLSSV |
| | VTVPSSSLGTKTYTCNVDHKPSNTKVDKRVESKYGPPC |
| | PPCPAPEFLGGPSVFLFPPKPKDTLMISRTPEVTCVVVD |
| | VSQEDPEVQFNWYVDGVEVHNAKTKPREEQFNSTYRV |
| | VSVLTVLHQDWLNGKEYKCKVSNKGLPSSIEKTISKAKG |
| | QPREPQVYTLPPSQEEMTKNQVSLTCLVKGFYPSDIAV |
| | EWESNGQPENNYKTTPPVLDSDGSFFLYSRLTVDKSR |
| | WQEGNVFSCSVMHEALHNHYTQKSLSLSLGK (SEQ ID |
| | NO: 3) |
| Pembrolizumab, | EIVLTQSPATLSLSPGERATLSCRASKGVSTSGYSYLHW |
| MK3475, full length light | YQQKPGQAPRLLIYLASYLESGVPARFSGSGSGTDFTLT |
| chain | ISSLEPEDFAVYYCQHSRDLPLTFGGGTKVEIKRTVAAP |
| From WO 2009/114335 | SVFIFPPSDEQLKSGTASVVCLLNNFYPREAKVQWKVD |
| | NALQSGNSQESVTEQDSKDSTYSLSSTLTLSKADYEKH |
| | KVYACEVTHQGLSSPVTKSFNRGEC (SEQ ID NO: 4) |
| mAb1 light chain variable | DIVMTQSPDSLAVSLGERATINCKSSQSLWDSGNQKNF |
| domain with CDRs in | LTWYQQKPGQPPKLLIYWTSTRESGVPDRFSGSGSGT |
| bold (from WO | DFTLTISSLQAEDVAVYYCQNDYFYPLTFGGGTKVEIK |
| 16/92419) | (SEQ ID NO: 5) |
| mAb1 heavy chain | QVQLVQSGAEVKKPGASVKVSCKASGYTFTSYWINWVRQ |
| variable domain with | APGQGLEQMGNIYPGSSLTNYNEKFKNRVTMTRDTSTST |
| CDRs underlined (from | VYMELSSLRSEDTAVYYCARLLTGTFAYWGQGTLVTVSS |
| WO 16/92419) | (SEQ ID NO: 6) |
| mAb2 light chain variable | DIVMTQSPDSLAVSLGERATINCKSSQSLWDSGNQKNFLT |
| domain with CDRs | WYQQKPGQPPKLLIYWTSYRESGVPDRFSGSGSGTDFTL |
| underlined (from WO | TISSLQAEDVAVYYCQNDYFYPLTFGGGTKVEIK (SEQ ID |
| 16/92419) | NO: 7) |
| mAb2 heavy chain | QVQLVQSGAEVKKPGASVKVSCKASGYTFTSYWINWVRQ |
| variable domain with | APGQGLEWMGNIYPGSSLTNYNEKFKNRVTMTRDTSTST |
| CDRs underlined (from | VYMELSSLRSEDTAVYYCARLLTGTFAYWGQGTLVTVSS |
| WO 16/92419) | (SEQ ID NO: 8) |
| mAb3 light chain variable | DIVMTQSPDSLAVSLGERATINCKSSQSLWDSGNQKNFLT |
| domain with CDRs | WYQQKPGQPPKLLIYWTSYRESGVPDRFSGSGSGTDFTL |
| underlined (from WO | TISSLQAEDVAVYYCQNDYFYPHTFGGGTKVEIK (SEQ ID |
| 16/92419) | NO: 9) |
| mAb3 heavy chain | QVQLVQSGAEVKKPGASVKVSCKASGYTFTSYWINWVRQ |
| variable domain with | APGQGLEWMGNIYPGSSLTNYNEKFKNRVTMTRDTSTST |
| CDRs underlined (from | VYMELSSLRSEDTAVYYCARLLTGTFAYWGQGTLVTVSS |
| WO 16/92419) | (SEQ ID NO: 10) |
| mAb4 light chain variable | DIVMTQSPDSLAVSLGERATINCKSSQSLWDSTNQKNFLT |
| domain with CDRs | WYQQKPGQPPKLLIYWTSTRESGVPDRFSGSGSGTDFTL |
| underlined (from WO | TISSLQAEDVAVYYCQNDYFYPLTFGGGTKVEIK (SEQ ID |
| 16/92419) | NO: 11) |
| mAb4 heavy chain | QVQLVQSGAEVKKPGASVKVSCKASGYTFTSYWINWVRQ |
| variable domain with | APGQGLEWMGNIYPGSSLTNYNEKFKNRVTMTRDTSTST |
| CDRs underlined (from | VYMELSSLRSEDTAVYYCARLLTGTFAYWGQGTLVTVSS |
| WO 16/92419) | (SEQ ID NO: 12) |
| mAb5 light chain variable | DIVTQSPDSLAVSLGERATINCKSSQSLWDSGNQKNFLT |
| domain with CDRs | WYQQKPGQPPKLLIYWTSTRESGVPDRFSGSGSGTDFTL |
| underlined (from WO | TISSLQAEDVAVYYCQNDYFYPLTFGGGTKVEIK (SEQ ID |
| 16/92419) | NO: 13) |
| mAb5 heavy chain | QVQLVWSGAEVKKPGASVKVSCKASGYTFTSYWINWVR |
| variable domain with | QAPGQGLEWMGNIYPGSSLTNYNEKFKNRVTMTRDTSTS |
| CDRs underlined (from | TVYMELSSLRSEDTAVYYCARLSTGTFAYWGQGTLVTVS |
| WO 16/92419) | S (SEQ ID NO: 14) |
| mAb6 light chain variable | DIVMTQSPDSLAVSLGERATINCKSSQSLWDSGNQKNFLT |
| domain with CDRs | WYQQKPGQPPKLLIYWTSYRESGVPDRFSGSGSGTDFTL |
| underlined (from WO | TISSLQAEDVAVYYCQNDYFYPLTFGGGTKVEIK (SEQ ID |
| 16/92419) | NO: 15) |
| mAb6 heavy chain | QVQLVQSGAEVKKPGASVKVSCKASGYTFTSYWINWVRQ |
| variable domain with | APGQGLEQMGNIYPGSSLTNYNEKFKNRVTMTRDTSTST |
| CDRs underlined (from | VYMELSSLRSEDTAVYYCARLSTGTFAYWGQGTLCTVSS |
| WO 16/92419) | (SEQ ID NO: 16) |
| mAb7(also known as | QVQLVQSGAEVKKPGASVKVSCKASGYTFTSYWINWVRQ |
| RN888) or mAb15 full- | APGQGLEWMGNIYPGSSLTNYNEKFKNRVTMTRDTSTST |
| length heavy chain | VYMELSSLRSEDTAVYYCARLSTGTFAYWGQGTLVTVSS |
| | ASTKGPSVFPLAPCSRSTSESTAALGCLVKDYFPEPVTVS |
| | WNSGALTSGVHTFPAVLQSSGLYSLSSVVTVPSSSLGTKT |
| | YTCNVDHKPSNTKVDKRVESKYGPPCPPCPAPEFLGGPS |
| | VFLFPPKPKDTLMISRTPEVTCVVVDVSQEDPEVQFNWYV |
| | DGVEVHNAKTKPREEQFNSTYRVVSVLTVLHQDWLNGKE |
| | YKCKVSNKGLPSSIEKTISKAKGQPREPQVYTLPPSQEEM |
| | TKNQVSLTCLVKGFYPSDIAVEWESNGQPENNYKTTPPVL |
| | DSDGSFFLYSRLTVDKSRWQEGNVFSCSVMHEALHNHYT |
| | QKSLSLSLGK (SEQ ID NO: 17) |
| mAb7 or mAb 15 full- | QVQLVQSGAEVKKPGASVKVSCKASGYTFTSYWINWVRQ |
| length heavy chain | APGQGLEWMGNIYPGSSLTNYNEKFKNRVTMTRDTSTST |
| without the C-terminal | VYMELSSLRSEDTAVYYCARLSTGTFAYWGQGTLVTVSS |
| lysine | ASTKGPSVFPLAPCSRSTSESTAALGCLVKDYFPEPVTVS |
| | WNSGALTSGVHTFPAVLQSSGLYSLSSVVTVPSSSLGTKT |
| | YTCNVDHKPSNTKVDKRVESKYGPPCPPCPAPEFLGGPS |
| | VFLFPPKPKDTLMISRTPEVTCVVVDVSQEDPEVQFNWYV |
| | DGVEVHNAKTKPREEQFNSTYRVVSVLTVLHQDWLNGKE |
| | YKCKVSNKGLPSSIEKTISKAKGQPREPQVYTLPPSQEEM |
| | TKNQVSLTCLVKGFYPSDIAVEWESNGQPENNYKTTPPVL |
| | DSDGSFFLYSRLTVDKSRWQEGNVFSCSVMHEALHNHYT |
| | QKSLSLSLG (SEQ ID NO: 18) |
| mAb7 full-length light | DIVMTQSPDSLAVSLGERATINCKSSQSLWDSGNQKNFLT |
| chain | WYQQKPGQPPKLLIYWTSYRESGVPDRFSGSGSGTDFTL |
| | TISSLQAEDVAVYYCQNDYFYPHTFGGGTKVEIKRGTVAA |
| | PSVFIFPPSDEQLKSGTASVVCLLNNFYPREAKVQWKVDN |
| | ALQSGNSQESVTEQDSKDSTYSLSSTLTLSKADYEKHKVY |
| | ACEVTHQGLSSPVTKSFNRGEC (SEQ ID NO: 19) |
| mAb7 light chain variable | QVQLVQSGAEVKKPGASVKVSCKASGYTFTSYWINWVRQ |
| region | APGQGLEWMGNIYPGSSLTNYNEKFKNRVTMTRDTSTST |
| | VYMELSSLRSEDTAVYYCARLSTGTFAYWGQGTLVTVSS |
| | (SEQ ID NO: 20) |
| mAB7 and mAB15 heavy | QVQLVQSGAEVKKPGASVKVSCKASGYTFTSYWINWVRQ |
| chain variable region | APGQGLEWMGNIWPGSSLTNYNEKFKNRVTMTRDTSTST |
| | VYMELSSLRSEDTAVYYCARLLTGTFAYWGQGTLVTVSS |
| | (SEQ ID NO: 21) |
| mAb8 light chain variable | DIVTQSPDSLAVSLGERATINCKSSQSLWDSTNQKNFLTW |
| domain with CDRs in | YQQKPGQPPKLLIYWTSTRESGVPDRFSGSGSGTDFTLTI |
| bold (from WO | SSLQAEDVAVYYCQNDYFYPLTFGGGTKVEIK (SEQ ID |
| 16/92419) | NO: 22) |
| mAb8 heavy chain | QVQLVQSGAEVKKPGASVKVSCKASGYTFTSYWINWVRQ |
| variable domain with | APGQGLEWMGNIYPGSSLTNYNEKFKNRVTMTRDTSTST |
| CDRs in bold (from WO | VYMELSSLRSEDTAVYYCARLSTGTFAYWGQTLVTVSS |
| 16/92419) | (SEQ ID NO: 23) |
| mAb9 light chain variable | DIVMTQSPDSLAVSLGERATINCKSSQSLWDSGNQKNF |
| domain with CDRs in | LTWYQQKPGQPPKLLIYWTSTRESGVPDRFSGSGSGT |
| bold (from WO | DFTLTISSLQAEDVAVYYCQNDYFYPLTFGGGTKVEIK |
| 16/92419) | (SEQ ID NO: 24) |
| mAb9 heavy chain | QVQLVQSGAEVKKPGASVKVSCKASGYTFTSYWINWVRQ |
| variable domain with | APGQGLEWMGNIYPGSSITNYNEKFKNRVTMTRDTSTST |
| CDRs in bold (from WO | VYMELSSLRSEDTAVYYCARLTTGTFAYWGQGTLVTVSS |
| 16/92419) | (SEQ ID NO: 25) |
| mAb10 light chain | DIVMTQSPDSLAVSLGERATINCKSSQSLWDSGNQKNFLT |
| variable domain with | WYQQKPGQPPKLLIYWTSYRESGVPDRFSGSGSGTDFTL |
| CDRs in bold (from WO | TISSLQAEDVAVYYCQNDYFYPLTFGGGTKVEIK (SEQ ID |
| 16/92419) | NO: 26) |
| mAb10 heavy chain | QVQLVQSGAEVKKPGASVKVSCKASGYTFTSYWINWVRQ |
| variable domain with | APGQGLEWMGNIYPGSSITNYNEKFKNRVTMTRDTSTST |
| CDRs in bold (from WO | VYMELSSLRSEDTAVYYCARLTTGTFAYWGQGTLVTVSS |
| 16/92419) | (SEQ ID NO: 27) |
| mAb11 light chain | DIVMTQSPDSLAVSLGERATINCKSSQSLWDSGNQKNFLT |
| variable domain with | WYQQKPGQPPKLLIYWTSYRESGVPDRFSGSGSGTDFTL |
| CDRs in bold (from WO | TISSLQAEDVAVYYCQNDYFYPHTFGGGTKVEIK (SEQ ID |
| 16/92419) | NO: 28) |
| mAb11 heavy chain | QVQLVQSGAEVKKPGASVKVSCKASGYTFTSYWINWVRQ |
| variable domain with | APGQGLEWMGNIYPGSSITNYNEKFKNRVTMTRDTSTST |
| CDRs in bold (from WO | VYMELSSLRSEDTAVYYCARLTTGTFAYWGQGTLVTVSS |
| 16/92419) | (SEQ ID NO: 29) |
| mAb12 light chain | DIVMTQSPDSLAVSLGERATINCKSSQSLWDSTNQKNFLT |
| variable domain with | WYQQKPGQPPKLLIYWTSTRESGVPDRFSGSGSGTDFTL |
| CDRs in bold (from WO | TISSLQAEDVAVYYCQNDYFYPLTFGGGTKVEIK (SEQ ID |
| 16/92419) | NO: 30) |
| mAb12 heavy chain | QVQLVQSGAEVKKPGASVKVSCKASGYTFTSYWINWVRQ |
| variable domain with | APGQGLEWMGNIYPGSSITNYNEKFKNRVTMTRDTSTST |
| CDRs in bold (from WO | VYMELSSLRSEDTAVYYCARLTTGTFAYWGQGTLVTVSS |
| 16/92419) | (SEQ ID NO: 31) |
| mAb13 light chain | DIVMTQSPDSLAVSLGERATINCKSSQSLWDSGNQKNFLT |
| variable domain with | WYQQKPGQPPKLLIYWTSTRESGVPDRFSGSGSGTDFTL |
| CDRs in bold (from WO | TISSLQAEDVAVYYCQNDYFYPLTFGGGTKVEIK (SEQ ID |
| 16/92419) | NO: 32) |
| mAb13 heavy chain | QVQLVQSGAEVKKPGASVKVSCKASGYTFTSYWINWVRQ |
| variable domain with | APGQGLEWMGNIWPGSSLTNYNEKFKNRVTMTRDTSTS |
| CDRs in bold (from WO | TVYMELSSLRSEDTAVYYCARLLTGTFAYWGQGTLVTVS |
| 16/92419) | S (SEQ ID NO: 33) |
| mAb14 light chain | DIVMTQSPDSLAVSLGERATINCKSSQSLWDSGNQKNFLT |
| variable domain with | WYQQKPGQPPKLLIYWTSYRESGVPDRFSGSGSGTDFTL |
| CDRs in bold (from WO | TISSLQAEDVAVYYCQNDYFYPLTFGGGTKVEIK (SEQ ID |
| 16/92419) | NO: 34) |
| mAb14 heavy chain | QVQLVQSGAEVKKPGASVKVSCKASGYTFTSYWINWVRQ |
| variable domain with | APGQGLEWMGNIWPGSSLTNYNEKFKNRVTMTRDTSTS |
| CDRs in bold (from WO | TVYMELSSLRSEDTAVYYCARLLTGTFAYWGQGTLVTVS |
| 16/92419) | S (SEQ ID NO: 35) |
| mAb15 light chain | DIVMTQSPDSLAVSLGERATINCKSSQSLWDSGNQKNFLT |
| variable region | WYQQKPGQPPKLLIYWTSYRESGVPDRFSGSGSGTDFTL |
| | TISSLQAEDVAVYYCQNDYFYPHTFGGGTKVEIK (SEQ ID |
| | NO: 36) |
| mAb16 light chain | DIVMTQSPDSLAVSLGERATINCKSSQSLWDSTNQKNFLT |
| variable domain with | WYQQKPGQPPKLLIYWTSTRESGVPDRFSGSGSGTDFTL |
| CDRs in bold (from WO | TISSLQAEDVAVYYCQNDYFYPLTFGGGTKVEIK (SEQ ID |
| 16/92419) | NO: 37) |
| mAb16 heavy chain | QVQLVQSGAEVKKPGASVKVSCKASGYTFTSYWINWVRQ |
| variable domain with | APGQGLEWMGNIWPGSSLTNYNEKFKNRVTMTRDTSTS |
| CDRs in bold (from WO | TVYMELSSLRSEDTAVYYCARLLTGTFAYWGQGTLVTVS |
| 16/92419) | S (SEQ ID NO: 38) |
| AMP224, without signal | LFTVTVPKELYIIEHGSNVTLECNFDTGSHVNLGAITASLQK |
| sequence | VENDTSPHRERATLLEEQLPLGKASFHIPQVQVRDEGQYQ |
| From WO 2010/027827 | CIIIYGVA |
| and WO 2011/066342 | WDYKYLTLKVKASYRKINTHILKVPETDEVELTCQATGYPL |
| | AEVSWPNVSVPANTSHSRTPEGLYQVTSVLRLKPPPGRN |
| | FSCVFWNTHVRELTLASIDLQSQMEPRTHPTWEPKSCDK |
| | THTCPPCPAPELLGGPSVFLFPPKPKDTLMISRTPEVTCW |
| | VDVSHEDPEVKFNWYVDGVEVHNAKTKPREEQYNSTYR |
| | WSVLTVLHQDWLNGKEYKCKVSNKALPAPIEKTISKAKGQ |
| | PREPQVYTLPPSRDELTKNQVSLTCLVKGFY |
| | PSDIAVEWES NGQPENNYKT TPPVLDSDGS |
| | FFLYSKLTVDKSRWQQGNVFSCSVMHEALHNHYTQKSLS |
| | LSPGK (SEQ ID NO: 39) |

Further examples of an anti-PD-1 antibody include CT-011 (pidilizumab, which is described in WO 09/101611), IBI-308, atezolizumab (MPDL3280A), MED14736, avelumab, BMS 936559, ANB011 (TSR-042), mDX-400, BGB-108, MEDI-0680, SHR-1210, PF-06801591, PDR-001, GB-226, STI-1110, MEDI-0680 (AMP-514), PDR001, REGN2810, BGB-108, and BGB-A317, or a biosimilar of any of these antibodies. Additional exemplary anti-PD-1 antibodies are described in U.S. Patent Application Nos. 2017/0349666, 2017/0327590, 2017/0327582, 2017/0320949, 2017/0290913, 2017/0290808, 2017/0275705, 2017/0274073, 2017/0267762, 2017/0247456, 2017/0239351, 2017/0239351, 2017/0198037, 2017/0166641, 2017/0121409, 2017/0112925, 2017/0112925, 2017/0088615, 2017/0052188, 2017/0044260, 2017/0044256, 2017/0039345, 2017/0037127, 2017/0037125, 2017/0021019, 2017/0008971, 2017/0000885, 2016/0376367, 2016/0362492, 2016/0312297, 2016/0312295, 2016/0304969, 2016/0304606, 2016/0303231, 2016/0289315, 2016/0257752, 2016/0222118, 2016/0222113, 2016/0206719, 2016/0206719, 2016/0193334, 2016/0166685, 2016/0158360, 2016/0130348, 2016/0130345, 2016/0075783, 2016/0068586, 2016/0052990, 2016/0051672, 2016/0039903, 2016/0031990, 2016/0022814, 2016/0002334, 2015/0265705, 2015/0250837, 2015/0232555, 2015/0216970, 2015/0210772, 2015/0210769, 2015/0203579 2015/0190506, 2015/0152180, 2015/0125955, 2015/0118245, 2015/0118234, 2015/0071910, 2014/0356363, 2014/0348743, 2014/0341902, 2014/0335093, 2014/0294852, 2014/0271684, 2014/0234296, 2014/0227262, 2014/0178370, 2014/0044738, 2013/0230514, 2013/0164294, 2013/0156774, 2013/0133091, 2013/0109843, 2013/0108651, 2013/0095098, 2013/0022629, 2013/0022600, 2012/0114649, 2012/0114648, 2012/0039906, 2011/0280878, 2011/0229461, 2011/0195068, 2011/0171220, 2011/0171215, 2011/0159023, 2011/0123550, 2011/0008777, 2011/0008369, 2010/0285013, 2010/0266617, 2010/0055102, 2009/0263865, 2009/0217401, 2009/0076250, 2009/0028857, 2008/0311117, 2008/0025979, 2007/0092504, 2006/0210567, 2006/0034826, 2004/0241745, and 2004/0213795.

In some embodiments, a PD-1 binding antagonist can be a fusion protein (e.g., an immunoadhesin, e.g., AMP-224, also called B7-DCIg, which is described in WO 10/027827 and WO 11/066342). For example, an immunoadhesin can include an extracellular or PD-1 binding portion of PD-L1 or PD-L2 fused to an antibody constant region (e.g., an Fc region of an immunoglobulin (e.g., a human immunoglobulin) sequence).

In some embodiments, a PD-1 binding antagonist can be an aptamer. Non-limiting examples of PD-1 binding antagonists that are aptamers are described in, e.g., US 2017/0218369. Additional examples of aptamers that are PD-1 binding antagonists are described in Prodeus et al., Mol. Ther. Nucleic Acids 4:e237, 2015; Wang et al., doi: 10.1016/j.biochi.2017.09.006 Biochimie. For example, a PD-1 binding antagonist that is an aptamer can include a sequence of one of:

(SEQ ID NO: 40)
GCTACTGTACATCACGCCTCTCCCC,
(SEQ ID NO: 41)
CTACTGTACATCACGCCTCTCCCC,
(SEQ ID NO: 42)
GTACAGTTCCCGTCCCTGCACTACA,
or
(SEQ ID NO: 43)
GTACAGTTCCCGTCCTGCACTACA.

The CSF-1R inhibitor in the combination therapies of the invention is ARRY-382 or pharmaceutically acceptable salts thereof. ARRY-382 has the following structure:

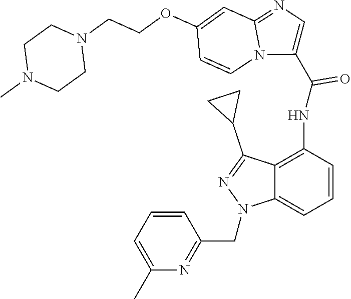 [SMILES: Cc1cccc(Cn2nc(C3CC3)c3c(NC(=O)c4cnc5cc(OCCN6CCN(C)CC6)ccn45)cccc32)n1]

ARRY-382 is also known as N-(3-cyclopropyl-1-((6-methylpyridin-2-yl)methyl)-1H-indazol-4-yl)-7-(2-(4-methylpiperazin-1-yl)ethoxy)imidazo[1,2-a]pyridine-3-carboxamide. Methods of preparing ARRY-382 and its pharmaceutically acceptable salts are described in PCT publication No. WO 2011/079076, in Example 65, the disclosure of which is herein incorporated by reference in its entirety. In one embodiment, the ARRY-382 is a free base. In one embodiment, ARRY-382 is a pharmaceutically acceptable salt.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer (including metastatic non-small cell lung cancer, PD-L1 positive non-small cell lung cancer and advanced or metastatic PD-L1 positive non-small cell lung cancer), glioma, Hodgkin's lymphoma, non-Hodgkin's lymphoma, acute myeloid leukemia (AML), multiple myeloma, gastrointestinal (tract) cancer, renal cancer (including advanced renal cell carcinoma), ovarian cancer, liver cancer, lymphoblastic leukemia, lymphocytic leukemia, colorectal cancer (including metastatic colorectal cancer, such as microsatellite stable metastatic colorectal cancer), endometrial cancer, kidney cancer, prostate cancer, thyroid cancer, melanoma (including advanced, unresectable or metastatic melanoma, including BRAF V600 mutant melanoma, such as BRAF V600E mutant melanoma), chondrosarcoma, neuroblastoma, pancreatic cancer (including microsatellite stable (MSS)/mismatch repair (MMR)-proficient pancreatic ductal adenocarcinoma), glioblastoma multiforme, cervical cancer, brain cancer, stomach cancer, urothelial carcinoma (including local advanced or metastatic urothelial carcinoma), bladder cancer, hepatoma, breast cancer (including triple negative breast cancer), colon carcinoma, head and neck cancer (including head and neck squamous cell carcinoma), mesothelioma, and uterine cancer. In one embodiment, the cancer is pancreatic cancer. In one embodiment, the pancreatic cancer is MMS/MMR-proficient pancreatic ductal adenocarcinoma. In one embodiment, the cancer is ovarian cancer. In one embodiment, the cancer is colorectal cancer. In one embodiment, the cancer is metastatic colorectal cancer. In one embodiment, the cancer is gastric cancer. In one embodiment, the cancer is melanoma. In one embodiment, the cancer is advanced, unresectable or metastatic melanoma. In one embodiment, the cancer is breast cancer. In one embodiment, the cancer is triple negative breast cancer. In one embodiment, the cancer is bladder cancer. In one embodiment, the cancer is non-small cell lung cancer. In one embodiment, the cancer is advanced or metastatic PD-L1 positive non-small cell lung cancer. In one embodiment, the advanced or metastatic PD-L1 positive non-small cell lung cancer does not have a EGFR or ALK genomic aberration. In one embodiment, the advanced or metastatic PD-L1 positive non-small cell lung cancer does not have an EGFR or ALK genomic aberration and the patient has not received prior systemic chemotherapy. In one embodiment, the advanced or metastatic PD-L1 positive non-small cell lung cancer does not have an EGFR or ALK genomic aberration and the patient had disease progression on or after platinum-containing chemotherapy. In one embodiment, the advanced or metastatic PD-L1 positive non-small cell lung cancer has a EGFR or ALK genomic aberrations. In one embodiment, the advanced or metastatic PD-L1 positive non-small cell lung cancer has an EGFR or ALK genomic aberrations and the patient had disease progression on a targeted therapy for EGFR or ALK genomic tumor aberrations (i.e., an EGFR or ALK inhibitor) such as erlotinib, osimertinib, or certinib.

The term “combination therapy” as used herein refers to a dosing regimen of at least two different therapeutically active agents (i.e., the components or combination partners of the combination) (e.g., ARRY-382 or a pharmaceutically acceptable salt thereof and a PD-1 binding antagonist) during a period of time, wherein the therapeutically active agents are administered together or separately in a manner prescribed by a medical care taker or according to a regulatory agency as defined herein. In one embodiment, a combination therapy comprises combination of a CSF-1R inhibitor which is ARRY-382 or a pharmaceutically acceptable salt thereof, and a PD-1 binding antagonist which is nivolumab or a biosimilar thereof. In one embodiment, a combination therapy comprises a combination of a CSF-1R inhibitor which is ARRY-382 or a pharmaceutically acceptable salt thereof, and a PD-1 binding antagonist which is pembrolizumab or a biosimilar thereof.

As can be appreciated in the art, a combination therapy can be administered to a patient for a period of time. In some embodiments, the period of time occurs following the administration of a different cancer therapeutic treatment/agent or a different combination of cancer therapeutic treatments/agents to the patient. In some embodiments, the period of time occurs before the administration of a different cancer therapeutic treatment/agent or a different combination of cancer therapeutic treatments/agents to the patient. In some embodiments, administration of the PD-1 binding antagonist and administration of ARRY-382 or a pharmaceutically acceptable salt thereof occurs at substantially the same time. In some embodiments, administration of the PD-1 binding antagonist to the patient occurs prior to administration of ARRY-382 or a pharmaceutically acceptable salt thereof to the patient, during the period of time. In some embodiments, administration of ARRY-382 or a pharmaceutically acceptable salt thereof to the patient occurs prior to administration of the PD-1 binding antagonist to the patient, during the period of time. In some embodiments, the patient is administered a surgical treatment (e.g., tumor resection and/or lymph node resection) and/or chemotherapy (e.g., a chemotherapeutic agent that does not include a BRAF kinase inhibitor, e.g., encorafenib) during the period of time. In some embodiments, the patient is not administered a BRAF kinase inhibitor (e.g., encorafenib) during the period of time.

A suitable period of time can be determined by one skilled in the art (e.g., a physician). As can be appreciated in the art, a suitable period of time can be determined by one skilled in the art based on one or more of: the stage of disease in the patient, the mass and sex of the patient, clinical trial guidelines (e.g., those on the fda.gov website), and information on the approved drug label. For example a suitable period of time can be, e.g., from 1 week to 2 years, 1 week to 22 months, 1 week to 20 months, 1 week to 18 months, 1 week to 16 months, 1 week to 14 months, 1 week to 12 months, 1 week to 10 months, 1 week to 8 months, 1 week to 6 months, 1 week to 4 months 1 week to 2 months, 1 week to 1 month, 2 weeks to 2 years, 2 weeks to 22 months, 2 weeks to 20 months, 2 weeks to 18 months, 2 weeks to 16 months, 2 weeks to 14 months, 2 weeks to 12 months, 2 weeks to 10 months, 2 weeks to 8 months, 2 weeks to 6 months, 2 weeks to 4 months, 2 weeks to 2 months, 2 weeks to 1 month, 1 month to 2 years, 1 month to 22 months, 1 month to 20 months, 1 month to 18 months, 1 month to 16 months, 1 month to 14 months, 1 month to 12 months, 1 month to 10 months, 1 month to 8 months, 1 month to 6 months, 1 month to 4 months, 1 month to 2 months, 2 months to 2 years, 2 months to 22 months, 2 months to 20 months, 2 months to 18 months, 2 months to 16 months, 2 months to 14 months, 2 months to 12 months, 2 months to 10 months, 2 months to 8 months, 2 months to 6 months, 2 months to 4 months, 3 months to 2 years, 3 months to 22 months, 3 months to 20 months, 3 months to 18 months, 3 months to 16 months, 3 months to 14 months, 3 months to 12 months, 3 months to 10 months, 3 months to 8 months, 3 months to 6 months, 4 months to 2 years, 4 months to 22 months, 4 months to 20 months, 4 months to 18 months, 4 months to 16 months, 4 months to 14 months, 4 months to 12 months, 4 months to 10 months, 4 months to 8 months, 4 months to 6 months, 6 months to 2 years, 6 months to 22 months, 6 months to 20 months, 6 months to 18 months, 6 months to 16 months, 6 months to 14 months, 6 months to 12 months, 6 months to 10 months, 6 months to 8 months, 8 months to 2 years, 8 months to 22 months, 8 months to 20 months, 8 months to 18 months, 8 months to 16 months, 8 months to 14 months, 8 months to 12 months, 8 months to 10 months, 10 months to 2 years, 10 months to 22 months, 10 months to 20 months, 10 months to 18 months, 10 months to 16 months, 10 months to 14 months, 10 months to 12 months, 12 months to 2 years, 12 months to 22 months, 12 months to 20 months, 12 months to 18 months, 12 months to 16 months, or 12 months to 14 months, inclusive.

As used herein, an “effective dosage” or “effective amount” or “therapeutically effective amount” of a drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various diseases or conditions (such as for example cancer), decreasing the dose of other medications required to treat the disease, enhancing the effect of another medication, and/or delaying the progression of the disease. An effective dosage can be administered in one or more administrations. For purposes of this invention, an effective dosage of a drug, compound, or pharmaceutical composition is an amount sufficient to accomplish prophylactic or therapeutic treatment either directly or indirectly. As is understood in the clinical context, an effective dosage of a drug, compound, or pharmaceutical composition may be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved. In reference to the treatment of cancer, an effective amount may also refer to that amount which has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens may also be characterized as the ability to induce, enhance, maintain or prolong disease control and/or overall survival in patients with these specific tumors, which may be measured as prolongation of the time before disease progression

The term “Q2W” as used herein means once every two weeks.

The term “Q3W” as used herein means once every three weeks.

The term “BID” as used herein means twice a day.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

The term “advanced”, as used herein, as it relates to solid tumors, includes locally advanced (non-metastatic) disease and metastatic disease. Locally advanced solid tumors, which may or may not be treated with curative intent, and metastatic disease, which cannot be treated with curative intent, are included within the scope of “advanced solid tumors as used herein. Those skilled in the art will be able to recognize and diagnose advanced solid tumors in a patient.

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer.

In one embodiment, such benefit includes any one or more of: extending survival (including overall survival and/or progression-free survival); resulting in an objective response (including a complete response or a partial response); or improving signs or symptoms of cancer. Additional methods of determining an effective response are described herein.

An “objective response” or “OR” refers to a measurable response, including complete response (CR) or partial response (PR). An “objective response rate” (ORR) refers to the proportion of patients with tumor size reduction of a predefined amount and for a minimum time period. Generally, ORR refers to the sum of complete response (CR) rate and partial response (PR) rate.

“Complete response” or “CR” as used herein means the disappearance of all signs of cancer (e.g., disappearance of all target lesions) in response to treatment. This does not always mean the cancer has been cured.

As used herein, “partial response” or “PR” refers to a decrease in the size of one or more tumors or lesions, or in the extent of cancer in the body, in response to treatment. For example, in some embodiments, PR refers to at least a 30% decrease in the sum of the longest diameters (SLD) of target lesions, taking as reference the baseline SLD.

“Sustained response” refers to the sustained effect on reducing tumor growth after cessation of a treatment. For example, the tumor size may be the same size or smaller as compared to the size at the beginning of the medicament administration phase. In some embodiments, the sustained response has a duration of at least the same as the treatment duration, at least 1.5×, 2×, 2.5×, or 3× length of the treatment duration, or longer.

As used herein, “progression-free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall survival” (OS) refers to the percentage of individuals in a group who are likely to be alive after a particular duration of time.

“Duration of Response” for purposes of the present invention means the time from documentation of tumor model growth inhibition due to drug treatment to the time of acquisition of a restored growth rate similar to pretreatment growth rate.

By “extending survival” is meant increasing overall or progression-free survival in a treated patient relative to an untreated patient (i.e. relative to a patient not treated with the medicament).

As used herein, “drug related toxicity”, “infusion related reactions” and “immune related adverse events” (“irAE”), and the severity or grades thereof are as exemplified and defined in the National Cancer Institute's Common Terminology Criteria for Adverse Events v 4.0 (NCI CTCAE v 4.0).

“Loss of heterozygosity score” or “LOH score” as used here in, refers to the percentage of genomic LOH in the tumor tissues of an individual. Percentage genomic LOH, and the calculation thereof are described in Swisher et al (The Lancet Oncology, 18(1):75-87, January 2017), the disclosure of which is incorporated herein by reference in its entirety. Exemplary genetic analysis includes, without limitation, DNA sequencing, and Foundation Medicine's NGS-based T5 assay.

“Homologous recombination deficiency score” or “HRD score” as used here in, refers to the unweighted numeric sum of loss of heterozygosity (“LOH”), telomeric allelic imbalance (“TAI”) and large-scale state transitions (“LST”) in the tumor tissues of an individual. HRD score, together with LOH, and LOH score, and the calculation thereof are described in Timms et al., Breast Cancer Res 2014 Dec. 5; 16(6):475, Telli et al., Clin Cancer Res; 22(15); 3764-73.2016, the disclosures of which are incorporated herein by reference in their entireties. Exemplary genetic analysis includes, without limitation, DNA sequencing, Myriad's HRD or HRD Plus assay (Mirza et al., N Engl J Med 2016 Dec. 1; 375(22):2154-2164, 2016).

The term “tumor proportion score” or “TPS” as used herein refers to the percentage of viable tumor cells showing partial or complete membrane staining in an immunohistochemistry test of a sample. “Tumor proportion score of PD-L1 expression” as used here in refers to the percentage of viable tumor cells showing partial or complete membrane staining in a PD-L1 expression immunohistochemistry test of a sample. Exemplary samples include, without limitation, a biological sample, a tissue sample, a formalin-fixed paraffin-embedded (FFPE) human tissue sample and a formalin-fixed paraffin-embedded (FFPE) human tumor tissue sample. Exemplary PD-L1 expression immunohistochemistry tests include, without limitation, the PD-L1 IHC 22C3 PharmDx (FDA approved, Daco), Ventana PD-L1 SP263 assay, and the tests described in international patent application PCT/EP2017/073712.

In some embodiments, the anti-cancer effects of the methods described herein, including, but not limited to “objective response”, “complete response”, “partial response”, “progressive disease”, “stable disease”, “progression free survival”, “duration of response”, are as defined and assessed by the investigators using RECIST v1.1 (Eisenhauer et al., Eur J of Cancer 2009; 45(2):228-47) in patients with locally advanced or metastatic solid tumors other than metastatic castration-resistant prostate cancer (CRPC), and RECIST v1.1 and PCWG3 (Scher et al., J Clin Oncol 2016 Apr. 20; 34(12): 1402-18) in patients with metastatic CRPC. The disclosures of Eisenhauer et al., Eur J of Cancer 2009; 45(2):228-47 and Scher et al., J Clin Oncol 2016 Apr. 20; 34(12): 1402-18 are herein incorporated by references in their entireties.

In some embodiments, the anti-cancer effect of the methods described herein, including, but not limited to “immune-related objective response” (irOR), “immune-related complete response” (irCR), “immune-related partial response” (irCR), “immune-related progressive disease” (irPD), “immune-related stable disease” (irSD), “immune-related progression free survival” (irPFS), and “immune-related duration of response” (irDR), are as defined and assessed by Immune-related response criteria (irRECIST, Nishino et. al. J Immunother Cancer 2014; 2:17) for patients with locally advanced or metastatic solid tumors other than patients with metastatic CRPC. The disclosure of Nishino et. al. J Immunother Cancer 2014; 2:17 is herein incorporated by reference in its entirety.

As used herein, “in combination with” refers to the administration of a CSF-1R inhibitor which is ARRY-382 or a pharmaceutically acceptable salt thereof, and a PD-1 binding antagonist, concurrently, sequentially or intermittently as separate dosage.

The term “additive” is used to mean that the result of the combination of two components of the combination therapy is no greater than the sum of each compound, component or targeted agent individually. The term “additive” means that there is no improvement in the disease condition or disorder being treated over the use of each component individually.

The term “synergy” or “synergistic” is used herein to mean that the effect of the combination of the two therapeutic agents of the combination therapy is greater than the sum of the effect of each agent when administered alone. A “synergistic amount” or “synergistically effective amount” is an amount of the combination of the two combination partners that results in a synergistic effect, as “synergistic” is defined herein. Determining a synergistic interaction between two combination partners, the optimum range for the effect and absolute dose ranges of each component for the effect may be definitively measured by administration of the combination partners over different w/w (weight per weight) ratio ranges and doses to patients in need of treatment. However, the observation of synergy in in vitro models or in vivo models can be predictive of the effect in humans and other species and in vitro models or in vivo models exist, as described herein, to measure a synergistic effect and the results of such studies can also be used to predict effective dose and plasma concentration ratio ranges and the absolute doses and plasma concentrations required in humans and other species by the application of pharmacokinetic/pharmacodynamic methods. For example, art-accepted in vitro and animal models of cancers described herein are known in the art, and are described in the Examples. Exemplary synergistic effects include, but are not limited to, enhanced therapeutic, efficacy, decreased dosage at equal or increased level of efficacy, reduced or delayed development of drug resistance, and simultaneous enhancement or equal therapeutic actions and reduction of unwanted side effects.

For example, a synergistic ratio of two therapeutic agents can be identified by determining a synergistic effect in an art-accepted in vitro (e.g., cancer cell line) or in vivo (animal model) model of any of the cancers described herein. Non-limiting examples of cancer cell lines and in vivo animal models of the cancers described herein are described in the Examples. Additional examples of art-accepted cancer cell lines and in vivo animal models are known in the art.

In some embodiments, “synergistic effect” as used herein refers to combination of a CSF-1R inhibitor which is ARRY-382 or a pharmaceutically acceptable salt thereof, and a PD-1 binding antagonist producing an effect, for example, any of the beneficial or desired results including clinical results as described herein, for example slowing the symptomatic progression of a proliferative disease, particularly cancer, or symptoms thereof, which is greater than the sum of the effect observed when ARRY-382 or a pharmaceutically acceptable salt thereof and a PD-1 binding antagonist are administered alone.

In some embodiments, the methods provided herein can result in a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the volume of one or more solid tumors in a patient following treatment with the combination therapy for a period of time between 1 day and 2 years (e.g., between 1 day and 22 months, between 1 day and 20 months, between 1 day and 18 months, between 1 day and 16 months, between 1 day and 14 months, between 1 day and 12 months, between 1 day and 10 months, between 1 day and 9 months, between 1 day and 8 months, between 1 day and 7 months, between 1 day and 6 months, between 1 day and 5 months, between 1 day and 4 months, between 1 day and 3 months, between 1 day and 2 months, between 1 day and 1 month, between one week and 2 years, between 1 week and 22 months, between 1 week and 20 months, between 1 week and 18 months, between 1 week and 16 months, between 1 week and 14 months, between 1 week and 12 months, between 1 week and 10 months, between 1 week and 9 months, between 1 week and 8 months, between 1 week and 7 months, between 1 week and 6 months, between 1 week and 5 months, between 1 week and 4 months, between 1 week and 3 months, between 1 week and 2 months, between 1 week and 1 month, between 2 weeks and 2 years, between 2 weeks and 22 months, between 2 weeks and 20 months, between 2 weeks and 18 months, between 2 weeks and 16 months, between 2 weeks and 14 months, between 2 weeks and 12 months, between 2 weeks and 10 months, between 2 weeks and 9 months, between 2 weeks and 8 months, between 2 weeks and 7 months, between 2 weeks and 6 months, between 2 weeks and 5 months, between 2 weeks and 4 months, between 2 weeks and 3 months, between 2 weeks and 2 months, between 2 weeks and 1 month, between 1 month and 2 years, between 1 month and 22 months, between 1 month and 20 months, between 1 month and 18 months, between 1 month and 16 months, between 1 month and 14 months, between 1 month and 12 months, between 1 month and 10 months, between 1 month and 9 months, between 1 month and 8 months, between 1 month and 7 months, between 1 month and 6 months, between 1 month and 6 months, between 1 month and 5 months, between 1 month and 4 months, between 1 month and 3 months, between 1 month and 2 months, between 2 months and 2 years, between 2 months and 22 months, between 2 months and 20 months, between 2 months and 18 months, between 2 months and 16 months, between 2 months and 14 months, between 2 months and 12 months, between 2 months and 10 months, between 2 months and 9 months, between 2 months and 8 months, between 2 months and 7 months, between 2 months and 6 months, or between 2 months and 5 months, between 2 months and 4 months, between 3 months and 2 years, between 3 months and 22 months, between 3 months and 20 months, between 3 months and 18 months, between 3 months and 16 months, between 3 months and 14 months, between 3 months and 12 months, between 3 months and 10 months, between 3 months and 8 months, between 3 months and 6 months, between 4 months and 2 years, between 4 months and 22 months, between 4 months and 20 months, between 4 months and 18 months, between 4 months and 16 months, between 4 months and 14 months, between 4 months and 12 months, between 4 months and 10 months, between 4 months and 8 months, between 4 months and 6 months, between 6 months and 2 years, between 6 months and 22 months, between 6 months and 20 months, between 6 months and 18 months, between 6 months and 16 months, between 6 months and 14 months, between 6 months and 12 months, between 6 months and 10 months, or between 6 months and 8 months) (e.g., as compared to the size of the one or more solid tumors in the patient prior to treatment).

In some embodiments, any of the methods described herein can provide for a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the risk of developing a metastasis or the risk of developing an additional metastasis in a patient having a cancer.

The phrase “time of survival” means the length of time between the identification or diagnosis of cancer (e.g., any of the cancers described herein) in a mammal by a medical professional and the time of death of the mammal (caused by the cancer). Methods of increasing the time of survival in a mammal having a cancer are described herein.

In some embodiments, any of the methods described herein can result in an increase (e.g., a 1% to 400%, 1% to 380%, 1% to 360%, 1% to 340%, 1% to 320%, 1% to 300%, 1% to 280%, 1% to 260%, 1% to 240%, 1% to 220%, 1% to 200%, 1% to 180%, 1% to 160%, 1% to 140%, 1% to 120%, 1% to 100%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 5% to 400%, 5% to 380%, 5% to 360%, 5% to 340%, 5% to 320%, 5% to 300%, 5% to 280%, 5% to 260%, 5% to 240%, 5% to 220%, 5% to 200%, 5% to 180%, 5% to 160%, 5% to 140%, 5% to 120%, 5% to 100%, 5% to 90%, 5% to 80%, 5% to 70%, 5% to 60%, 5% to 50%, 5% to 40%, 5% to 30%, 5% to 20%, 5% to 10%, 10% to 400%, 10% to 380%, 10% to 360%, 10% to 340%, 10% to 320%, 10% to 300%, 10% to 280%, 10% to 260%, 10% to 240%, 10% to 220%, 10% to 200%, 10% to 180%, 10% to 160%, 10% to 140%, 10% to 120%, 10% to 100%, 10% to 90%, 10% to 80%, 10% to 70%, 10% to 60%, 10% to 50%, 10% to 40%, 10% to 30%, 10% to 20%, 20% to 400%, 20% to 380%, 20% to 360%, 20% to 340%, 20% to 320%, 20% to 300%, 20% to 280%, 20% to 260%, 20% to 240%, 20% to 220%, 20% to 200%, 20% to 180%, 20% to 160%, 20% to 140%, 20% to 120%, 20% to 100%, 20% to 90%, 20% to 80%, 20% to 70%, 20% to 60%, 20% to 50%, 20% to 40%, 20% to 30%, 30% to 400%, 30% to 380%, 30% to 360%, 30% to 340%, 30% to 320%, 30% to 300%, 30% to 280%, 30% to 260%, 30% to 240%, 30% to 220%, 30% to 200%, 30% to 180%, 30% to 160%, 30% to 140%, 30% to 120%, 30% to 100%, 30% to 90%, 30% to 80%, 30% to 70%, 30% to 60%, 30% to 50%, 30% to 40%, 40% to 400%, 40% to 380%, 40% to 360%, 40% to 340%, 40% to 320%, 40% to 300%, 40% to 280%, 40% to 260%, 40% to 240%, 40% to 220%, 40% to 200%, 40% to 180%, 40% to 160%, 40% to 140%, 40% to 120%, 40% to 100%, 40% to 90%, 40% to 80%, 40% to 70%, 40% to 60%, 40% to 50%, 50% to 400%, 50% to 380%, 50% to 360%, 50% to 340%, 50% to 320%, 50% to 300%, 50% to 280%, 50% to 260%, 50% to 240%, 50% to 220%, 50% to 200%, 50% to 180%, 50% to 160%, 50% to 140%, 50% to 140%, 50% to 120%, 50% to 100%, 50% to 90%, 50% to 80%, 50% to 70%, 50% to 60%, 60% to 400%, 60% to 380%, 60% to 360%, 60% to 340%, 60% to 320%, 60% to 300%, 60% to 280%, 60% to 260%, 60% to 240%, 60% to 220%, 60% to 200%, 60% to 180%, 60% to 160%, 60% to 140%, 60% to 120%, 60% to 100%, 60% to 90%, 60% to 80%, 60% to 70%, 70% to 400%, 70% to 380%, 70% to 360%, 70% to 340%, 70% to 320%, 70% to 300%, 70% to 280%, 70% to 260%, 70% to 240%, 70% to 220%, 70% to 200%, 70% to 180%, 70% to 160%, 70% to 140%, 70% to 120%, to 100%, 70% to 90%, 70% to 80%, 80% to 400%, 80% to 380%, 80% to 360%, 80% to 340%, 80% to 320%, 80% to 300%, 80% to 280%, 80% to 260%, 80% to 240%, 80% to 220%, 80% to 200%, 80% to 180%, 80% to 160%, 80% to 140%, 80% to 120%, 80% to 100%, 80% to 90%, 90% to 400%, 90% to 380%, 90% to 360%, 90% to 340%, 90% to 320%, 90% to 300%, 90% to 280%, 90% to 260%, 90% to 240%, 90% to 220%, 90% to 200%, 90% to 180%, 90% to 160%, 90% to 140%, 90% to 120%, 90% to 100%, 100% to 400%, 100% to 380%, 100% to 360%, 100% to 340%, 100% to 320%, 100% to 300%, 100% to 280%, 100% to 260%, 100% to 240%, 100% to 220%, 100% to 200%, 100% to 180%, 100% to 160%, 100% to 140%, 100% to 120%, 120% to 400%, 120% to 380%, 120% to 360%, 120% to 340%, 120% to 320%, 120% to 300%, 120% to 280%, 120% to 260%, 120% to 240%, 120% to 220%, 120% to 200%, 120% to 180%, 120% to 160%, 120% to 140%, 140% to 400%, 140% to 380%, 140% to 360%, 140% to 340%, 140% to 320%, 140% to 300%, 140% to 280%, 140% to 260%, 140% to 240%, 140% to 220%, 140% to 200%, 140% to 180%, 140% to 160%, 160% to 400%, 160% to 380%, 160% to 360%, 160% to 340%, 160% to 320%, 160% to 300%, 160% to 280%, 160% to 260%, 160% to 240%, 160% to 220%, 160% to 200%, 160% to 180%, 180% to 400%, 180% to 380%, 180% to 360%, 180% to 340%, 180% to 320%, 180% to 300%, 180% to 280%, 180% to 260%, 180% to 240%, 180% to 220%, 180% to 200%, 200% to 400%, 200% to 380%, 200% to 360%, 200% to 340%, 200% to 320%, 200% to 300%, 200% to 280%, 200% to 260%, 200% to 240%, 200% to 220%, 220% to 400%, 220% to 380%, 220% to 360%, 220% to 340%, 220% to 320%, 220% to 300%, 220% to 280%, 220% to 260%, 220% to 240%, 240% to 400%, 240% to 380%, 240% to 360%, 240% to 340%, 240% to 320%, 240% to 300%, 240% to 280%, 240% to 260%, 260% to 400%, 260% to 380%, 260% to 360%, 260% to 340%, 260% to 320%, 260% to 300%, 260% to 280%, 280% to 400%, 280% to 380%, 280% to 360%, 280% to 340%, 280% to 320%, 280% to 300%, 300% to 400%, 300% to 380%, 300% to 360%, 300% to 340%, or 300% to 320%) in the time of survival of the patient (e.g., as compared to a patient having a similar cancer and administered a different treatment or not receiving a treatment).

As used herein, the term “cytokine” refers generically to proteins released by one cell population that act on another cell as intercellular mediators or have an autocrine effect on the cells producing the proteins. Examples of such cytokines include lymphokines, monokines; interleukins (“ILs”) such as IL-1, IL-1a, IL-2, IL-3, IL-4, IL-5, IL-6, IL-7, IL-8, IL-9, IL10, IL-11, IL-12, IL-13, IL-15, IL-17A-F, IL-18 to IL-29 (such as IL-23), IL-31, including PROLEUKIN® rIL-2; a tumor-necrosis factor such as TNF-a or TNF-β, TGF-I-3; and other polypeptide factors including leukemia inhibitory factor (“LIF”), ciliary neurotrophic factor (“CNTF”), CNTF-like cytokine (“CLC”), cardiotrophin (“CT”), and kit ligand (“L”).

As used herein, the term “chemokine” refers to soluble factors (e.g., cytokines) that have the ability to selectively induce chemotaxis and activation of leukocytes. They also trigger processes of angiogenesis, inflammation, wound healing, and tumorigenesis. Example chemokines include IL-8, a human homolog of murine keratinocyte chemoattractant (KC).

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. As used herein, the singular form “a”, “an”, and “the” include plural references unless indicated otherwise. For example, “an” excipient includes one or more excipients. It is understood that aspects and variations of the invention described herein include “consisting of” and/or “consisting essentially of” aspects and variations.

Exemplary methods and materials are described herein, although methods and materials similar or equivalent to those described herein can also be used in the practice or testing of the invention. The materials, methods, and examples are illustrative only and not intended to be limiting.

### Methods, Uses, and Medicaments

In one embodiment, an amount of a CSF-1R inhibitor which is ARRY-382 or a pharmaceutically acceptable salt thereof, is used in combination with an amount of a PD-1 binding antagonist, wherein the amounts together are effective in the treatment of cancer.

In one embodiment, a therapeutically effective amount of each of the combination partners of a combination therapy of the invention are administered separately and may be administered simultaneously, sequentially, or intermittently, and in any order, at specific or varying time intervals.

In one embodiment, provided herein is a method of treating a proliferative disease, including cancer, which comprises or consists essentially of administering, during a period of time, a combination therapy comprising, consisting essentially of, or consisting of a CSF-1R inhibitor which is ARRY-382 or a pharmaceutically acceptable salt thereof, and a PD-1 binding antagonist, to a patient in need thereof, wherein the individual combination partners are administered in jointly therapeutically effective amounts, (for example in synergistically effective amounts). The individual combination partners of a combination therapy of the invention may be administered in daily or intermittent dosages during the period of time. The individual combination partners of a combination therapy of the invention may be administered separately at different times and in any order during the period of time, or concurrently in divided combination forms during the period of time. In one embodiment, the CSF-1R inhibitor, which is ARRY-382 or a pharmaceutically acceptable salt thereof, is administered on a daily basis, either once daily or twice daily, during the period of time. In one embodiment, the CSF-1R inhibitor which is ARRY-382 or a pharmaceutically acceptable salt thereof is administered twice daily on a daily basis during the period of time. In one embodiment, the PD-1 binding antagonist is administered on a weekly basis during the period of time. In one embodiment, the PD-1 binding antagonist is administered every 2 weeks (Q2W) during the period of time. In one embodiment, the PD-1 binding antagonist is administered every 3 weeks (Q3W) during the period of time. In one embodiment, the PD-1 binding antagonist is administered every 4 weeks (Q4W), during the period of time. In one embodiment, the PD-1 binding antagonist is administered every 8 weeks (Q8W), during the period of time. In one embodiment, the PD-1 binding antagonist is administered every 4 weeks (Q4W) and the CSF-1R inhibitor, which is ARRY-382 or a pharmaceutically acceptable salt thereof, is administered on a daily basis, during the period of time. In some embodiments, the administration of the inhibitor of the PD-1 binding antagonist starts on the same day as the administration of the CSF-1R inhibitor, which is ARRY-382 or a pharmaceutically acceptable salt thereof. In some embodiments, the PD-1 binding antagonist is administered after the CSF-1R inhibitor, which is ARRY-382 or a pharmaceutically acceptable salt thereof, has been administered for about four weeks or more. The instant invention is therefore to be understood as embracing all such regimens of simultaneous or alternating treatment during the period of time and the term “administering” is to be interpreted accordingly.

The term “jointly therapeutically effective amount” as used herein means when the therapeutic agents of a combination described herein are given to the patient simultaneously or separately (e.g., in a chronologically staggered manner, for example a sequence-specific manner) in such time intervals that they show an interaction (e.g., a joint therapeutic effect, for example a synergistic effect). Whether this is the case can, inter alia, be determined by following the blood levels and showing that the combination components are present in the blood of the human to be treated at least during certain time intervals.

In one embodiment, provided herein is a method of treating a subject having a proliferative disease comprising, consisting essentially of, or consisting of administering to said subject a combination therapy as described herein, during a period of time, in a quantity which is jointly therapeutically effective against a proliferative disease. In one embodiment, the proliferative disease is cancer. In one embodiment, the cancer is selected from squamous cell carcinoma, myeloma, small-cell lung cancer, non-small cell lung cancer (including metastatic non-small cell lung cancer, PD-L1 positive non-small cell lung cancer and advanced or metastatic PD-L1 positive non-small cell lung cancer), glioma, Hodgkin's lymphoma, non-Hodgkin's lymphoma, acute myeloid leukemia (AML), multiple myeloma, gastrointestinal (tract) cancer, renal cancer (including advanced renal cell carcinoma), ovarian cancer, liver cancer, lymphoblastic leukemia, lymphocytic leukemia, colorectal cancer (including metastatic colorectal cancer, such as microsatellite stable metastatic colorectal cancer), endometrial cancer, kidney cancer, prostate cancer, thyroid cancer, melanoma (including advanced, unresectable or metastatic melanoma, including BRAF V600 mutant melanoma, such as BRAF V600E mutant melanoma), chondrosarcoma, neuroblastoma, pancreatic cancer (including microsatellite stable (MSS)/mismatch repair (MMR)-proficient pancreatic ductal adenocarcinoma), glioblastoma multiforme, cervical cancer, brain cancer, stomach cancer, urothelial carcinoma (including local advanced or metastatic urothelial carcinoma), bladder cancer, hepatoma, breast cancer (including triple negative breast cancer), colon carcinoma, head and neck cancer (including head and neck squamous cell carcinoma), mesothelioma, and uterine cancer.

In one embodiment, the cancer is selected from non-small cell lung cancer, pancreatic cancer, ovarian cancer, colorectal cancer, gastric cancer, melanoma, breast cancer, bladder cancer, non-small cell lung cancer, head and neck cancer, uterine cancer, cervical cancer, liver cancer, thyroid cancer, kidney cancer, brain cancer, skin cancer, and mesothelioma.

In one embodiment, the cancer is pancreatic cancer. In one embodiment, the pancreatic cancer is MMS/MMR-proficient pancreatic ductal adenocarcinoma. In one embodiment, the cancer is ovarian cancer. In one embodiment, the cancer is colorectal cancer. In one embodiment, the cancer is metastatic colorectal cancer. In one embodiment, the cancer is gastric cancer. In one embodiment, the cancer is melanoma. In one embodiment, the cancer is advanced, unresectable or metastatic melanoma. In one embodiment, the cancer is breast cancer. In one embodiment, the cancer is triple negative breast cancer. In one embodiment, the cancer is bladder cancer. In one embodiment, the cancer is non-small cell lung cancer. In one embodiment, the cancer is advanced or metastatic PD-L1 positive non-small cell lung cancer. In one embodiment, the advanced or metastatic PD-L1 positive non-small cell lung cancer does not have a EGFR or ALK genomic aberration. In one embodiment, the advanced or metastatic PD-L1 positive non-small cell lung cancer does not have a EGFR or ALK genomic aberration and the patient has not received prior systemic chemotherapy. In one embodiment, the advanced or metastatic PD-L1 positive non-small cell lung cancer does not have a EGFR or ALK genomic aberration and the patient had disease progression on or after platinum-containing chemotherapy. In one embodiment, the advanced or metastatic PD-L1 positive non-small cell lung cancer has a EGFR or ALK genomic aberrations. In one embodiment, the advanced or metastatic PD-L1 positive non-small cell lung cancer has a EGFR or ALK genomic aberrations and the patient had disease progression on a US Food and Drug Administration-approved therapy for EGFR or ALK genomic tumor aberrations.

In some embodiments, the patient was treated, before the period of time, with one or more therapeutic agents that do not include a combination of a PD-1 binding antagonist and ARRAY-382 or a pharmaceutically acceptable salt thereof, e.g., at least one treatment with another anticancer treatment, e.g., treatment with chemotherapy, one more targeted therapeutic agents, or radiation therapy, or surgery.

The term “chemotherapy” as used herein refers to a chemotherapeutic agent, or a combination of two, three, four, or more chemotherapeutic agents, for the treatment of cancer. When a chemotherapy consists more than one chemotherapeutic agents, the chemotherapeutic agents can be administered to the patient on the same day or on different days in the same treatment cycle.

A “chemotherapeutic agent” or “chemotherapy” is a chemical compound useful in the treatment of cancer. Examples of chemotherapeutic agents include alkylating agents such as thiotepa and cyclophosphamide (CYTOXAN®); alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB 1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g. calicheamicin, especially calicheamicin gamma I I and calicheamicin omegal I (see, e.g., Nicolaou et ai, Angew. Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HC1 liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and fluoropyrimidine-containing chemotherapies such as 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative); anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDIS1NE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel, also known as nab-paclitaxel (ABRAXANE™), and docetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylomithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, FOLFOX, an abbreviation for a treatment regimen with oxaliplatin (ELOXATIN™) combined with 5-FU and leucovovin, and FOLFOXIRI (an abbreviation for a treatment regimen with folinic acid, 5-fluorouracil, oxaliplatin and irinotecan).

Additional examples of chemotherapeutic agents include anti-hormonal agents that act to regulate, reduce, block, or inhibit the effects of hormones that can promote the growth of cancer, and are often in the form of systemic or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY 1 1 7018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRFI) agonists such as leuprolide acetate (LUPRON® and ELIGARD®), goserelin acetate, buserelin acetate and tripterelin; anti-androgens such as fiutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RJVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); anti-sense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); an anti-estrogen such as fulvestrant; a Kit inhibitor such as imatinib or EXEL-0862 (a tyrosine kinase inhibitor); EGFR inhibitor such as erlotinib or cetuximab; an anti-VEGF inhibitor such as bevacizumab; irinotecan; rmRH (e.g., ABARELIX®); lapatinib and lapatinib ditosylate (an ErbB-2 and EGFR dual tyrosine kinase small-molecule inhibitor also known as GW572016); 17AAG (geldanamycin derivative that is a heat shock protein (Hsp) 90 poison), and pharmaceutically acceptable salts, acids or derivatives of any of the above.

A “platinum-based chemotherapy” as used herein, refers to a chemotherapy wherein at least one chemotherapeutic agent is a coordination complex of platinum. Exemplary platinum-based chemotherapy includes, without limitation, cisplatin, carboplatin, oxaliplatin, nedaplatin, gemcitabine in combination with cisplatin, carboplatin in combination with pemetrexed.

A “targeted therapeutic agent” as used herein includes, refers to a molecule that blocks the growth of cancer cells by interfering with specific targeted molecules needed for carcinogenesis and tumor growth, rather than by simply interfering with all rapidly dividing cells (e.g. with traditional chemotherapy), and includes but is not limited to, receptor tyrosine kinase-targeted therapeutic agents (for example cabozantinib, crizotinib, erlotinib, gefitinib, imatinib, lapatinib, nilotinib, pazopanib, pertuzumab, regorafenib, sunitinib, and trastuzumab), signal transduction pathway inhibitors (for example, Ras-Raf-MEK-ERK pathway inhibitors (e.g. sorafenib, trametinib, vemurafenib), PI3K-Akt-mTOR-S6K pathway inhibitors (e.g. everolimus, rapamycin, perifosine, temsirolimus) and modulators of the apoptosis pathway (e.g. obataclax)), and angiogenesis-targeted therapies (for example, aflibercept and bevacizumab).

In one embodiment, the one or more therapeutic agents that were administered to the patient before the period of time is chemotherapy.

In one embodiment, the one or more therapeutic agents that were administered to the patient before the period of time is paclitaxel.

In one embodiment, the one or more therapeutic agents that were administered to the patient before the period of time is selected from one or more of a platinum-based chemotherapy and a fluoropyrimidine-containing chemotherapy.

In one embodiment, the one or more therapeutic agents that were administered to the patient before the period of time is a platinum-based chemotherapy (e.g., gemcitabine).

In one embodiment, the one or more therapeutic agents that were administered to the patient before the period of time is a fluoropyrimidine-containing chemotherapy (e.g., fluorouracil (5-FU)).

In one embodiment, the one or more therapeutic agents that were administered to the patient before the period of time is FOLFIRINOX (a chemotherapy treatment regimen of folinic acid (leucovorin), fluorouracil (5-FU), irinotecan, and oxaliplatin).

In one embodiment, the one or more therapeutic agents that were administered to the patient before the period of time is FOLFOXIRI (a chemotherapy treatment regimen of folinic acid, 5-fluorouracil, oxaliplatin and irinotecan).

In some embodiments of any of the methods described herein, the one or more therapeutic agents that were administered to the patient before the period of time was unsuccessful (e.g., therapeutically unsuccessful as determined by a physician).

In one embodiment, the cancer is refractory to standard of care.

In one embodiment, the cancer has progressed after prior treatment with a platinum-based chemotherapy.

In one embodiment, the patient has pancreatic ductal adenocarcinoma and has been treated with one or more therapeutic agents prior to the period of time, and optionally, the prior treatment was determined to be unsuccessful. In one embodiment, the cancer is stage III pancreatic ductal adenocarcinoma. In one embodiment, the one or more therapeutic agents that were administered to the subject before the period of time was one or both of gemcitabine and paclitaxel. In one embodiment, the one or more therapeutic agents that were administered to the patient before the period of time comprises FOLFOXIRI. In one embodiment, the one or more therapeutic agents that were administered to the patient before the period of time comprises FOLFOX. In one embodiment, the combination therapy comprises administration of a CSF-1R inhibitor which is ARRY-382 or a pharmaceutically acceptable salt thereof and a PD-1 binding antagonist which is pembrolizumab or a biosimilar thereof. In one embodiment, the combination therapy comprises oral administration of ARRY-382 once a day on a daily basis (during the period of time), e.g., in an amount of about 100 mg to about 400 mg (e.g., about 10 mg to about 380 mg, about 10 mg to about 360 mg, about 10 mg to about 340 mg, about 10 mg to about 320 mg, about 10 mg to about 300 mg, about 10 mg to about 280 mg, about 10 mg to about 260 mg, about 10 mg to about 240 mg, about 10 mg to about 220 mg, about 10 mg to about 200 mg, about 10 mg to about 180 mg, about 10 mg to about 160 mg, about 10 mg to about 140 mg, about 10 mg to about 120 mg, about 10 mg to about 100 mg, about 10 mg to about 80 mg, about 10 mg to about 60 mg, about 10 mg to about 40 mg, about 10 mg to about 20 mg, about 20 mg to about 400 mg, about 20 mg to about 380 mg, about 20 mg to about 360 mg, about 20 mg to about 340 mg, about 20 mg to about 320 mg, about 20 mg to about 300 mg, about 20 mg to about 280 mg, about 20 mg to about 260 mg, about 20 mg to about 240 mg, about 20 mg to about 220 mg, about 20 mg to about 200 mg, about 20 mg to about 180 mg, about 20 mg to about 160 mg, about 20 mg to about 140 mg, about 20 mg to about 120 mg, about 20 mg to about 100 mg, about 20 mg to about 80 mg, about 20 mg to about 60 mg, about 20 mg to about 40 mg, about 40 mg to about 400 mg, about 40 mg to about 380 mg, about 40 mg to about 360 mg, about 40 mg to about 340 mg, about 40 mg to about 320 mg, about 40 mg to about 300 mg, about 40 mg to about 280 mg, about 40 mg to about 260 mg, about 40 mg to about 240 mg, about 40 mg to about 220 mg, about 40 mg to about 200 mg, about 40 mg to about 180 mg, about 40 mg to about 160 mg, about 40 mg to about 140 mg, about 40 mg to about 120 mg, about 40 mg to about 100 mg, about 40 mg to about 80 mg, about 40 mg to about 60 mg, about 60 mg to about 400 mg, about 60 mg to about 380 mg, about 60 mg to about 360 mg, about 60 mg to about 340 mg, about 60 mg to about 320 mg, about 60 mg to about 300 mg, about 60 mg to about 280 mg, about 60 mg to about 260 mg, about 60 mg to about 240 mg, about 60 mg to about 220 mg, about 60 mg to about 200 mg, about 60 mg to about 180 mg, about 60 mg to about 160 mg, about 60 mg to about 140 mg, about 60 mg to about 120 mg, about 60 mg to about 100 mg, about 60 mg to about 80 mg, about 80 mg to about 400 mg, about 80 mg to about 380 mg, about 80 mg to about 360 mg, about 80 mg to about 340 mg, about 80 mg to about 320 mg, about 80 mg to about 300 mg, about 80 mg to about 280 mg, about 80 mg to about 260 mg, about 80 mg to about 240 mg, about 80 mg to about 220 mg, about 80 mg to about 200 mg, about 80 mg to about 180 mg, about 80 mg to about 160 mg, about 80 mg to about 140 mg, about 80 mg to about 120 mg, about 80 mg to about 100 mg, about 100 mg to about 400 mg, about 100 mg to about 380 mg, about 100 mg to about 360 mg, about 100 mg to about 340 mg, about 100 mg to about 320 mg, about 100 mg to about 300 mg, about 100 mg to about 280 mg, about 100 mg to about 260 mg, about 100 mg to about 240 mg, about 100 mg to about 220 mg, about 100 mg to about 200 mg, about 100 mg to about 180 mg, about 100 mg to about 160 mg, about 100 mg to about 140 mg, about 100 mg to about 120 mg, about 120 mg to about 400 mg, about 120 mg to about 380 mg, about 120 mg to about 360 mg, about 120 mg to about 340 mg, about 120 mg to about 320 mg, about 120 mg to about 300 mg, about 120 mg to about 280 mg, about 120 mg to about 260 mg, about 120 mg to about 240 mg, about 120 mg to about 220 mg, about 120 mg to about 200 mg, about 120 mg to about 180 mg, about 120 mg to about 160 mg, about 120 mg to about 140 mg, about 140 mg to about 400 mg, about 140 mg to about 380 mg, about 140 mg to about 360 mg, about 140 mg to about 340 mg, about 140 mg to about 320 mg, about 140 mg to about 300 mg, about 140 mg to about 280 mg, about 140 mg to about 260 mg, about 140 mg to about 240 mg, about 140 mg to about 220 mg, about 140 mg to about 200 mg, about 140 mg to about 180 mg, about 140 mg to about 160 mg, about 160 mg to about 400 mg, about 160 mg to about 380 mg, about 160 mg to about 360 mg, about 160 mg to about 360 mg, about 160 mg to about 340 mg, about 160 mg to about 320 mg, about 160 mg to about 300 mg, about 160 mg to about 280 mg, about 160 mg to about 260 mg, about 160 mg to about 240 mg, about 160 mg to about 220 mg, about 160 mg to about 200 mg, about 160 mg to about 180 mg, about 180 mg to about 400 mg, about 180 mg to about 380 mg, about 180 mg to about 360 mg, about 180 mg to about 340 mg, about 180 mg to about 320 mg, about 180 mg to about 300 mg, about 180 mg to about 280 mg, about 180 mg to about 260 mg, about 180 mg to about 240 mg, about 180 mg to about 220 mg, about 180 mg to about 200 mg, about 200 mg to about 400 mg, about 200 mg to about 380 mg, about 200 mg to about 360 mg, about 200 mg to about 340 mg, about 200 mg to about 320 mg, about 200 mg to about 300 mg, about 200 mg to about 280 mg, about 200 mg to about 260 mg, about 200 mg to about 240 mg, about 200 mg to about 220 mg, about 220 mg to about 400 mg, about 220 mg to about 380 mg, about 220 mg to about 360 mg, about 220 mg to about 340 mg, about 220 mg to about 320 mg, about 220 mg to about 300 mg, about 220 mg to about 280 mg, about 220 mg to about 260 mg, about 220 mg to about 240 mg, about 240 mg to about 400 mg, about 240 mg to about 380 mg, about 240 mg to about 360 mg, about 240 mg to about 340 mg, about 240 mg to about 320 mg, about 240 mg to about 300 mg, about 240 mg to about 280 mg, about 240 mg to about 260 mg, about 260 mg to about 400 mg, about 260 mg to about 380 mg, about 260 mg to about 360 mg, about 260 mg to about 340 mg, about 260 mg to about 320 mg, about 260 mg to about 300 mg, about 260 mg to about 280 mg, about 280 mg to about 400 mg, about 280 mg to about 380 mg, about 280 mg to about 360 mg, about 280 mg to about 340 mg, about 280 mg to about 320 mg, about 280 mg to about 300 mg, about 300 mg to about 400 mg, about 300 mg to about 380 mg, about 300 mg to about 360 mg, about 300 mg to about 340 mg, about 300 mg to about 320 mg, about 320 mg to about 400 mg, about 320 mg to about 380 mg, about 320 mg to about 360 mg, about 340 mg to about 360 mg, about 340 mg to about 400 mg, about 340 mg to about 380 mg, about 340 mg to about 360 mg, about 360 mg to about 400 mg, about 360 mg to about 380 mg, about 380 mg to about 400 mg, about 100 mg, about 200 mg, about 300 mg, or about 400 mg), and intravenous administration of between about 1.0 mg/kg to about 5.0 mg/kg (e.g., about 1.0 mg/kg to about 4.5 mg/kg, about 1.0 mg/kg to about 4.0 mg/kg, about 1.0 mg/kg to about 3.5 mg/kg, about 1.0 mg/kg to about 3.0 mg/kg, about 1.0 mg/kg to about 2.5 mg/kg, about 1.0 mg/kg to about 2.0 mg/kg, about 1.0 mg/kg to about 1.5 mg/kg, about 1.5 mg/kg to about 5.0 mg/kg, about 1.5 mg/kg to about 4.5 mg/kg, about 1.5 mg/kg to about 4.0 mg/kg, about 1.5 mg/kg to about 3.5 mg/kg, about 1.5 mg/kg to about 3.0 mg/kg, about 1.5 mg/kg to about 2.5 mg/kg, about 1.5 mg/kg to about 2.0 mg/kg, about 2.0 mg/kg to about 5.0 mg/kg, about 2.0 mg/kg to about 4.5 mg/kg, about 2.0 mg/kg to about 4.0 mg/kg, about 2.0 mg/kg to about 3.5 mg/kg, about 2.0 mg/kg to about 3.0 mg/kg, about 2.0 mg/kg to about 2.5 mg/kg, about 2.5 mg/kg to about 5.0 mg/kg, about 2.5 mg/kg to about 4.5 mg/kg, about 2.5 mg/kg to about 4.0 mg/kg, about 2.5 mg/kg to about 3.5 mg/kg, about 2.5 mg/kg to about 3.0 mg/kg, about 3.0 mg/kg to about 5.0 mg/kg, about 3.0 mg/kg to about 4.5 mg/kg, about 3.0 mg/kg to about 4.0 mg/kg, about 3.0 mg/kg to about 3.5 mg/kg, about 3.5 mg/kg to about 5.0 mg/kg, about 3.5 mg/kg to about 4.5 mg/kg, about 3.5 mg/kg to about 4.0 mg/kg, about 4.0 mg/kg to about 5.0 mg/kg, about 4.0 mg/kg to about 4.5 mg/kg, about 4.5 mg/kg to about 5.0 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, or about 5.0 mg/kg) PD-1 binding antagonist (e.g., pembrolizumab or a biosimilar thereof, or nivolumab or a biosimilar thereof) every 1, 2, 3, or 4 weeks during the period of time.

In one embodiment, the patient has ovarian cancer and, before the period of time, the patient has been treated with one or more therapeutic agents, and optionally, the prior treatment with the one or more therapeutic agents was unsuccessful. In one embodiment, the cancer is stage IV (metastatic) ovarian cancer. In one embodiment, the cancer is metastatic ovarian cancer. In one embodiment, before the period of time, the patient has been administered one or more therapeutic agents selected from the group consisting of: paclitaxel, gemcitabine, carboplatin, cisplatin, and doxorubicin. In one embodiment, the combination therapy comprises administration of a CSF-1R inhibitor which is ARRY-382 or a pharmaceutically acceptable salt thereof and a PD-1 binding antagonist which is pembrolizumab or a biosimilar thereof, during the period of time. In one embodiment, the combination therapy comprises oral administration of ARRY-382 once a day on a daily basis in an amount of 200 mg, or 300 mg, or 400 mg, and intravenous administration of 2 mg/kg pembrolizumab or a biosimilar thereof every 3 weeks, during the period of time.

In one embodiment, before the period of time, the patient was administered one or more therapeutic agents that is an angiogenesis-targeted agent, e.g., an optionally, the prior treatment was not successful.

In one embodiment, before the period of time, the patient was administered surgery or a surgical treatment, and optionally, the surgery was not successful. Non-limiting examples of surgery include, e.g., open surgery or minimally invasive surgery. Surgery can include, e.g., removing an entire tumor, debulking of a tumor, or removing a tumor that is causing pain or pressure in the subject. Methods for performing open surgery and minimally invasive surgery on a subject having a cancer are known in the art. In some embodiments, surgery can include removal of at least one lymph node.

In one embodiment, before the period of time, the patient was administered radiotherapy, e.g., and optionally, the radiotherapy was not successful. Non-limiting examples of radiation therapy include external radiation beam therapy (e.g., external beam therapy using kilovoltage X-rays or megavoltage X-rays) or internal radiation therapy. Internal radiation therapy (also called brachytherapy) can include the use of, e.g., low-dose internal radiation therapy or high-dose internal radiation therapy. Low-dose internal radiation therapy includes, e.g., inserting small radioactive pellets (also called seeds) into or proximal to a cancer tissue in the subject. High-dose internal radiation therapy includes, e.g., inserting a thin tube (e.g., a catheter) or an implant into or proximal to a cancer tissue in the subject, and delivering a high dose of radiation to the thin tube or implant using a radiation machine. Methods for performing radiation therapy on a subject having a cancer are known in the art.

In some embodiments of any of the methods described herein, the patient has not been treated with an immune checkpoint inhibitor prior to the period of time. As used herein, the term “immune checkpoint inhibitor” or “checkpoint inhibitor” refers to molecules that totally or partially reduce, inhibit, interfere with, or modulate the expression and/or activity of one or more checkpoint proteins. In some embodiments, the immunotherapy includes one or more immune checkpoint inhibitors. In some embodiments, the immune checkpoint inhibitor is a CTLA-4 inhibitor (e.g., an anti-CTLA-4 antibody), a PD-1 inhibitor (e.g., PD-1 binding antagonist) or a PD-L1 inhibitor (e.g., an anti-PD-LI monoclonal antibody). In some embodiments, the CTLA-4 inhibitor is ipilimumab (Yervoy®) or tremelimumab (CP-675,206). In some embodiments, the PD-1 inhibitor is pembrolizumab (Keytruda®), nivolumab (Opdivo®), or pidilizumab. In some embodiments, the anti-PD-1 monoclonal antibody is nivolumab or pembrolizumab. In some embodiments, the anti-PD-1 antibody is pembrolizumab. In some embodiments, the PD-L1 inhibitor is atezolizumab (Tecentriq®), avelumab (Bavencio®), durvalumab (Imfinzi™), MED14736, or MPDL3280A. In some embodiments, the PD-L1 inhibitor is atezolizumab (Tecentriq®), avelumab (Bavencio®), or durvalumab (Imfinzi™). In some embodiments, a checkpoint inhibitor can target 4-1BB (e.g., urelumab (BMS-663513) and PF-05082566 (PF-2566)), CD27 (e.g., varlilumab (CDX-1127), CD40 (e.g., CP-870,893), OX40, TIM-3, ICOS, BTLA, A2AR, B7-H3, B7-H4, BTLA, IDO, KIR, LAG3, TIM-3, and VISTA. Additional non-limiting examples of immune checkpoint inhibitors include ulocuplumab, urelumab, PF 05082566, TRX518, varlilumab, CP 870893, PDR001MEDI4736, avelumab, BMS 986016, MGA271, IPH2201, emactuzumab, INCB024360, MED16469, galunisertib, BKT140, bavituximab, lirilumab, bevacizumab, MNRP1685A, lambroizumab, CC 90002, BMS-936559, and MGA271.

In one embodiment, the CSF-1R inhibitor is ARRY-382 as the free base. In one embodiment, the CSF-1R inhibitor is a pharmaceutically acceptable salt of ARRY-382. In one embodiment, ARRY-382 is orally administered during the period of time. In one embodiment, ARRY-382 is administered as a tablet during the period of time. In one embodiment, ARRY-382 is administered as a capsule during the period of time. In one embodiment, a capsule formulation of ARRY-382 comprises about 10 mg to about 100 mg (e.g., about 10 mg to about 95 mg, about 10 mg to about 90 mg, about 10 mg to about 85 mg, about 10 mg to about 80 mg, about 10 mg to about 75 mg, about 10 mg to about 70 mg, about 10 mg to about 65 mg, about 10 mg to about 60 mg, about 10 mg to about 55 mg, about 10 mg to about 50 mg, about 10 mg to about 45 mg, about 10 mg to about 40 mg, about 10 mg to about 35 mg, about 10 mg to about 30 mg, about 10 mg to about 25 mg, about 10 mg to about 20 mg, about 10 mg to about 15 mg, about 15 mg to about 100 mg, about 15 mg to about 95 mg, about 15 mg to about 90 mg, about 15 mg to about 85 mg, about 15 mg to about 80 mg, about 15 mg to about 75 mg, about 15 mg to about 70 mg, about 15 mg to about 65 mg, about 15 mg to about 60 mg, about 15 mg to about 55 mg, about 15 mg to about 50 mg, about 15 mg to about 45 mg, about 15 mg to about 40 mg, about 15 mg to about 35 mg, about 15 mg to about 30 mg, about 15 mg to about 25 mg, about 15 mg to about 20 mg, about 20 mg to about 100 mg, about 20 mg to about 95 mg, about 20 mg to about 90 mg, about 20 mg to about 85 mg, about 20 mg to about 80 mg, about 20 mg to about 75 mg, about 20 mg to about 70 mg, about 20 mg to about 65 mg, about 20 mg to about 60 mg, about 20 mg to about 55 mg, about 20 mg to about 50 mg, about 20 mg to about 45 mg, about 20 mg to about 40 mg, about 20 mg to about 35 mg, about 20 mg to about 30 mg, about 20 mg to about 25 mg, about 25 mg to about 100 mg, about 25 mg to about 95 mg, about 25 mg to about 90 mg, about 25 mg to about 85 mg, about 25 mg to about 80 mg, about 25 mg to about 75 mg, about 25 mg to about 70 mg, about 25 mg to about 65 mg, about 25 mg to about 60 mg, about 25 mg to about 55 mg, about 25 mg to about 50 mg, about 25 mg to about 45 mg, about 25 mg to about 40 mg, about 25 mg to about 35 mg, about 25 mg to about 30 mg, about 30 mg to about 100 mg, about 30 mg to about 95 mg, about 30 mg to about 90 mg, about 30 mg to about 85 mg, about 30 mg to about 80 mg, about 30 mg to about 75 mg, about 30 mg to about 70 mg, about 30 mg to about 65 mg, about 30 mg to about 60 mg, about 30 mg to about 55 mg, about 30 mg to about 50 mg, about 30 mg to about 45 mg, about 30 mg to about 40 mg, about 30 mg to about 35 mg, about 35 mg to about 100 mg, about 35 mg to about 95 mg, about 35 mg to about 90 mg, about 35 mg to about 85 mg, about 35 mg to about 80 mg, about 35 mg to about 75 mg, about 35 mg to about 70 mg, about 35 mg to about 65 mg, about 35 mg to about 60 mg, about 35 mg to about 55 mg, about 35 mg to about 50 mg, about 35 mg to about 45 mg, about 35 mg to about 40 mg, about 40 mg to about 100 mg, about 40 mg to about 95 mg, about 40 mg to about 90 mg, about 40 mg to about 85 mg, about 40 mg to about 80 mg, about 40 mg to about 75 mg, about 40 mg to about 70 mg, about 40 mg to about 65 mg, about 40 mg to about 60 mg, about 40 mg to about 55 mg, about 40 mg to about 50 mg, about 40 mg to about 45 mg, about 45 mg to about 100 mg, about 45 mg to about 95 mg, about 45 mg to about 90 mg, about 45 mg to about 85 mg, about 45 mg to about 80 mg, about 45 mg to about 75 mg, about 45 mg to about 70 mg, about 45 mg to about 65 mg, about 45 mg to about 60 mg, about 45 mg to about 55 mg, about 45 mg to about 50 mg, about 50 mg to about 100 mg, about 50 mg to about 95 mg, about 50 mg to about 90 mg, about 50 mg to about 85 mg, about 50 mg to about 80 mg, about 50 mg to about 75 mg, about 50 mg to about 70 mg, about 50 mg to about 65 mg, about 50 mg to about 60 mg, about 50 mg to about 55 mg, about 55 mg to about 100 mg, about 55 mg to about 95 mg, about 55 mg to about 90 mg, about 55 mg to about 85 mg, about 55 mg to about 80 mg, about 55 mg to about 75 mg, about 55 mg to about 70 mg, about 55 mg to about 65 mg, about 55 mg to about 60 mg, about 60 mg to about 100 mg, about 60 mg to about 95 mg, about 60 mg to about 90 mg, about 60 mg to about 85 mg, about 60 mg to about 80 mg, about 60 mg to about 75 mg, about 60 mg to about 70 mg, about 60 mg to about 65 mg, about 65 mg to about 100 mg, about 65 mg to about 95 mg, about 65 mg to about 90 mg, about 65 mg to about 85 mg, about 65 mg to about 80 mg, about 65 mg to about 75 mg, about 65 mg to about 70 mg, about 70 mg to about 100 mg, about 70 mg to about 95 mg, about 70 mg to about 90 mg, about 70 mg to about 85 mg, about 70 mg to about 80 mg, about 70 mg to about 75 mg, about 75 mg to about 100 mg, about 75 mg to about 95 mg, about 75 mg to about 90 mg, about 75 mg to about 85 mg, about 75 mg to about 80 mg, about 80 mg to about 100 mg, about 80 mg to about 95 mg, about 80 mg to about 90 mg, about 80 mg to about 85 mg, about 85 mg to about 100 mg, about 85 mg to about 95 mg, about 85 mg to about 90 mg, about 90 mg to about 100 mg, about 90 mg to about 95 mg, about 95 mg to about 100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg) of ARRY-382. In one embodiment, ARRY-382 is orally administered on a daily basis during the period of time. In one embodiment, ARRY-382 is orally administered in the amount of about 20 mg to about 500 mg (e.g., about 20 mg to about 480 mg, about 20 mg to about 460 mg, about 20 mg to about 440 mg, about 20 mg to about 420 mg, about 20 mg to about 400 mg, about 20 mg to about 380 mg, about 20 mg to about 360 mg, about 20 mg to about 340 mg, about 20 mg to about 320 mg, about 20 mg to about 300 mg, about 20 mg to about 280 mg, about 20 mg to about 260 mg, about 20 mg to about 240 mg, about 20 mg to about 220 mg, about 20 mg to about 200 mg, about 20 mg to about 180 mg, about 20 mg to about 160 mg, about 20 mg to about 140 mg, about 20 mg to about 120 mg, about 20 mg to about 100 mg, about 20 mg to about 80 mg, about 20 mg to about 60 mg, about 20 mg to about 40 mg, about 40 mg to about 500 mg, about 40 mg to about 480 mg, about 40 mg to about 460 mg, about 40 mg to about 440 mg, about 40 mg to about 420 mg, about 40 mg to about 400 mg, about 40 mg to about 380 mg, about 40 mg to about 360 mg, about 40 mg to about 340 mg, about 40 mg to about 320 mg, about 40 mg to about 300 mg, about 40 mg to about 280 mg, about 40 mg to about 260 mg, about 40 mg to about 240 mg, about 40 mg to about 220 mg, about 40 mg to about 200 mg, about 40 mg to about 180 mg, about 40 mg to about 160 mg, about 40 mg to about 140 mg, about 40 mg to about 120 mg, about 40 mg to about 100 mg, about 40 mg to about 80 mg, about 40 mg to about 60 mg, about 60 mg to about 500 mg, about 60 mg to about 480 mg, about 60 mg to about 460 mg, about 60 mg to about 440 mg, about 60 mg to about 420 mg, about 60 mg to about 400 mg, about 60 mg to about 380 mg, about 60 mg to about 360 mg, about 60 mg to about 340 mg, about 60 mg to about 320 mg, about 60 mg to about 300 mg, about 60 mg to about 280 mg, about 60 mg to about 260 mg, about 60 mg to about 240 mg, about 60 mg to about 220 mg, about 60 mg to about 200 mg, about 60 mg to about 180 mg, about 60 mg to about 160 mg, about 60 mg to about 140 mg, about 60 mg to about 120 mg, about 60 mg to about 100 mg, about 60 mg to about 80 mg, about 80 mg to about 500 mg, about 80 mg to about 480 mg, about 80 mg to about 460 mg, about 80 mg to about 440 mg, about 80 mg to about 420 mg, about 80 mg to about 400 mg, about 80 mg to about 380 mg, about 80 mg to about 360 mg, about 80 mg to about 340 mg, about 80 mg to about 320 mg, about 80 mg to about 300 mg, about 80 mg to about 280 mg, about 80 mg to about 260 mg, about 80 mg to about 240 mg, about 80 mg to about 220 mg, about 80 mg to about 200 mg, about 80 mg to about 180 mg, about 80 mg to about 160 mg, about 80 mg to about 140 mg, about 80 mg to about 120 mg, about 80 mg to about 100 mg, about 100 mg to about 500 mg, about 100 mg to about 480 mg, about 100 mg to about 460 mg, about 100 mg to about 440 mg, about 100 mg to about 420 mg, about 100 mg to about 400 mg, about 100 mg to about 380 mg, about 100 mg to about 360 mg, about 100 mg to about 340 mg, about 100 mg to about 320 mg, about 100 mg to about 300 mg, about 100 mg to about 280 mg, about 100 mg to about 260 mg, about 100 mg to about 240 mg, about 100 mg to about 220 mg, about 100 mg to about 200 mg, about 100 mg to about 180 mg, about 100 mg to about 160 mg, about 100 mg to about 140 mg, about 100 mg to about 120 mg, about 120 mg to about 500 mg, about 120 mg to about 480 mg, about 120 mg to about 460 mg, about 120 mg to about 440 mg, about 120 mg to about 420 mg, about 120 mg to about 400 mg, about 120 mg to about 380 mg, about 120 mg to about 360 mg, about 120 mg to about 340 mg, about 120 mg to about 320 mg, about 120 mg to about 300 mg, about 120 mg to about 280 mg, about 120 mg to about 260 mg, about 120 mg to about 240 mg, about 120 mg to about 220 mg, about 120 mg to about 200 mg, about 120 mg to about 180 mg, about 120 mg to about 160 mg, about 120 mg to about 140 mg, about 140 mg to about 500 mg, about 140 mg to about 480 mg, about 140 mg to about 460 mg, about 140 mg to about 440 mg, about 140 mg to about 420 mg, about 140 mg to about 400 mg, about 140 mg to about 380 mg, about 140 mg to about 360 mg, about 140 mg to about 340 mg, about 140 mg to about 320 mg, about 140 mg to about 300 mg, about 140 mg to about 280 mg, about 140 mg to about 260 mg, about 140 mg to about 240 mg, about 140 mg to about 220 mg, about 140 mg to about 200 mg, about 140 mg to about 180 mg, about 140 mg to about 160 mg, about 160 mg to about 500 mg, about 160 mg to about 480 mg, about 160 mg to about 460 mg, about 160 mg to about 440 mg, about 160 mg to about 420 mg, about 160 mg to about 400 mg, about 160 mg to about 380 mg, about 160 mg to about 360 mg, about 160 mg to about 340 mg, about 160 mg to about 320 mg, about 160 mg to about 300 mg, about 160 mg to about 280 mg, about 160 mg to about 260 mg, about 160 mg to about 240 mg, about 160 mg to about 220 mg, about 160 mg to about 200 mg, about 160 mg to about 180 mg, about 180 mg to about 500 mg, about 180 mg to about 480 mg, about 180 mg to about 460 mg, about 180 mg to about 440 mg, about 180 mg to about 420 mg, about 180 mg to about 400 mg, about 180 mg to about 380 mg, about 180 mg to about 360 mg, about 180 mg to about 340 mg, about 180 mg to about 320 mg, about 180 mg to about 300 mg, about 180 mg to about 280 mg, about 180 mg to about 260 mg, about 180 mg to about 240 mg, about 180 mg to about 220 mg, about 180 mg to about 200 mg, about 200 mg to about 500 mg, about 200 mg to about 480 mg, about 200 mg to about 460 mg, about 200 mg to about 440 mg, about 200 mg to about 420 mg, about 200 mg to about 400 mg, about 200 mg to about 380 mg, about 200 mg to about 360 mg, about 200 mg to about 340 mg, about 200 mg to about 320 mg, about 200 mg to about 300 mg, about 200 mg to about 280 mg, about 200 mg to about 260 mg, about 200 mg to about 240 mg, about 200 mg to about 220 mg, about 220 mg to about 500 mg, about 220 mg to about 480 mg, about 220 mg to about 460 mg, about 220 mg to about 440 mg, about 220 mg to about 420 mg, about 220 mg to about 400 mg, about 220 mg to about 380 mg, about 220 mg to about 360 mg, about 220 mg to about 340 mg, about 220 mg to about 320 mg, about 220 mg to about 300 mg, about 220 mg to about 280 mg, about 220 mg to about 260 mg, about 220 mg to about 240 mg, about 240 mg to about 500 mg, about 240 mg to about 480 mg, about 240 mg to about 460 mg, about 240 mg to about 440 mg, about 240 mg to about 420 mg, about 240 mg to about 400 mg, about 240 mg to about 380 mg, about 240 mg to about 360 mg, about 240 mg to about 340 mg, about 240 mg to about 320 mg, about 240 mg to about 300 mg, about 240 mg to about 280 mg, about 240 mg to about 260 mg, about 260 mg to about 500 mg, about 260 mg to about 480 mg, about 260 mg to about 460 mg, about 260 mg to about 440 mg, about 260 mg to about 420 mg, about 260 mg to about 400 mg, about 260 mg to about 380 mg, about 260 mg to about 360 mg, about 260 mg to about 340 mg, about 260 mg to about 320 mg, about 260 mg to about 300 mg, about 260 mg to about 280 mg, about 280 mg to about 500 mg, about 280 mg to about 480 mg, about 280 mg to about 460 mg, about 280 mg to about 440 mg, about 280 mg to about 420 mg, about 280 mg to about 400 mg, about 280 mg to about 380 mg, about 280 mg to about 360 mg, about 280 mg to about 340 mg, about 280 mg to about 320 mg, about 280 mg to about 300 mg, about 300 mg to about 500 mg, about 300 mg to about 480 mg, about 300 mg to about 460 mg, about 300 mg to about 440 mg, about 300 mg to about 420 mg, about 300 mg to about 400 mg, about 300 mg to about 380 mg, about 300 mg to about 360 mg, about 300 mg to about 340 mg, about 300 mg to about 320 mg, about 320 mg to about 500 mg, about 320 mg to about 480 mg, about 320 mg to about 460 mg, about 320 mg to about 440 mg, about 320 mg to about 420 mg, about 320 mg to about 400 mg, about 320 mg to about 380 mg, about 320 mg to about 360 mg, about 320 mg to about 340 mg, about 340 mg to about 500 mg, about 340 mg to about 480 mg, about 340 mg to about 460 mg, about 340 mg to about 440 mg, about 340 mg to about 420 mg, about 340 mg to about 400 mg, about 340 mg to about 380 mg, about 340 mg to about 360 mg, about 360 mg to about 500 mg, about 360 mg to about 480 mg, about 360 mg to about 460 mg, about 360 mg to about 440 mg, about 360 mg to about 420 mg, about 360 mg to about 400 mg, about 360 mg to about 380 mg, about 380 mg to about 500 mg, about 380 mg to about 480 mg, about 380 mg to about 460 mg, about 380 mg to about 440 mg, about 380 mg to about 420 mg, about 380 mg to about 400 mg, about 400 mg to about 500 mg, about 400 mg to about 480 mg, about 400 mg to about 460 mg, about 400 mg to about 440 mg, about 400 mg to about 420 mg, about 420 mg to about 500 mg, about 420 mg to about 480 mg, about 420 mg to about 460 mg, about 420 mg to about 440 mg, about 440 mg to about 500 mg, about 440 mg to about 480 mg, about 440 mg to about 460 mg, about 460 mg to about 500 mg, about 460 mg to about 480 mg, about 480 mg to about 500 mg, about 25, about 50, about 75, about 100, about 150, about 200, about 250, about 300, about 350, about 400, about 450, or about 500 mg), during the period of time. In one embodiment, ARRY-382 is orally administered once daily, during the period of time. In one embodiment, ARRY-382 is orally administered once daily in the amount of about 100 mg, during the period of time. In one embodiment, ARRY-382 is orally administered once daily in the amount of about 200 mg, during the period of time. In one embodiment, ARRY-382 is orally administered once daily in the amount of about 300 mg, during the period of time. In one embodiment, ARRY-382 is orally administered once daily in the amount of about 400 mg, during the period of time.

In some embodiments, the PD-1 binding antagonist is nivolumab. In one embodiment, nivolumab or a biosimilar thereof is administered, during the period of time, intravenously at a dose of about 1 mg/mg to about 40 mg/mg (e.g., about 1 mg/kg to about 38 mg/kg, about 1 mg/kg to about 36 mg/kg, about 1 mg/kg to about 34 mg/kg, about 1 mg/kg to about 32 mg/kg, about 1 mg/kg to about 30 mg/kg, about 1 mg/kg to about 28 mg/kg, about 1 mg/kg to about 26 mg/kg, about 1 mg/kg to about 24 mg/kg, about 1 mg/kg to about 22 mg/kg, about 1 mg/kg to about 20 mg/kg, about 1 mg/kg to about 18 mg/kg, about 1 mg/kg to about 16 mg/kg, about 1 mg/kg to about 14 mg/kg, about 1 mg/kg to about 12 mg/kg, about 1 mg/kg to about 10 mg/kg, about 1 mg/kg to about 8 mg/kg, about 1 mg/kg to about 6 mg/kg, about 1 mg/kg to about 4 mg/kg, about 2 mg/kg to about 40 mg/kg, about 2 mg/kg to about 38 mg/kg, about 2 mg/kg to about 36 mg/kg, about 2 mg/kg to about 34 mg/kg, about 2 mg/kg to about 32 mg/kg, about 2 mg/kg to about 30 mg/kg, about 2 mg/kg to about 28 mg/kg, about 2 mg/kg to about 26 mg/kg, about 2 mg/kg to about 24 mg/kg, about 2 mg/kg to about 22 mg/kg, about 2 mg/kg to about 20 mg/kg, about 2 mg/kg to about 18 mg/kg, about 2 mg/kg to about 16 mg/kg, about 2 mg/kg to about 14 mg/kg, about 2 mg/kg to about 12 mg/kg, about 2 mg/kg to about 10 mg/kg, about 2 mg/kg to about 8 mg/kg, about 2 mg/kg to about 6 mg/kg, about 2 mg/kg to about 4 mg/kg, about 4 mg/kg to about 40 mg/kg, about 4 mg/kg to about 38 mg/kg, about 4 mg/kg to about 36 mg/kg, about 4 mg/kg to about 34 mg/kg, about 4 mg/kg to about 32 mg/kg, about 4 mg/kg to about 30 mg/kg, about 4 mg/kg to about 28 mg/kg, about 4 mg/kg to about 26 mg/kg, about 4 mg/kg to about 24 mg/kg, about 4 mg/kg to about 22 mg/kg, about 4 mg/kg to about 20 mg/kg, about 4 mg/kg to about 18 mg/kg, about 4 mg/kg to about 16 mg/kg, about 4 mg/kg to about 14 mg/kg, about 4 mg/kg to about 12 mg/kg, about 4 mg/kg to about 10 mg/kg, about 4 mg/kg to about 8 mg/kg, about 4 mg/kg to about 6 mg/kg, about 6 mg/kg to about 40 mg/kg, about 6 mg/kg to about 38 mg/kg, about 6 mg/kg to about 36 mg/kg, about 6 mg/kg to about 34 mg/kg, about 6 mg/kg to about 32 mg/kg, about 6 mg/kg to about 30 mg/kg, about 6 mg/kg to about 28 mg/kg, about 6 mg/kg to about 26 mg/kg, about 6 mg/kg to about 24 mg/kg, about 6 mg/kg to about 22 mg/kg, about 6 mg/kg to about 20 mg/kg, about 6 mg/kg to about 18 mg/kg, about 6 mg/kg to about 16 mg/kg, about 6 mg/kg to about 14 mg/kg, about 6 mg/kg to about 12 mg/kg, about 6 mg/kg to about 10 mg/kg, about 6 mg/kg to about 8 mg/kg, about 8 mg/kg to about 40 mg/kg, about 8 mg/kg to about 38 mg/kg, about 8 mg/kg to about 36 mg/kg, about 8 mg/kg to about 34 mg/kg, about 8 mg/kg to about 32 mg/kg, about 8 mg/kg to about 30 mg/kg, about 8 mg/kg to about 28 mg/kg, about 8 mg/kg to about 26 mg/kg, about 8 mg/kg to about 24 mg/kg, about 8 mg/kg to about 22 mg/kg, about 8 mg/kg to about 20 mg/kg, about 8 mg/kg to about 18 mg/kg, about 8 mg/kg to about 16 mg/kg, about 8 mg/kg to about 14 mg/kg, about 8 mg/kg to about 12 mg/kg, about 8 mg/kg to about 10 mg/kg, about 10 mg/kg to about 40 mg/kg, about 10 mg/kg to about 38 mg/kg, about 10 mg/kg to about 36 mg/kg, about 10 mg/kg to about 34 mg/kg, about 10 mg/kg to about 32 mg/kg, about 10 mg/kg to about 30 mg/kg, about 10 mg/kg to about 28 mg/kg, about 10 mg/kg to about 26 mg/kg, about 10 mg/kg to about 24 mg/kg, about 10 mg/kg to about 22 mg/kg, about 10 mg/kg to about 20 mg/kg, about 10 mg/kg to about 18 mg/kg, about 10 mg/kg to about 16 mg/kg, about 10 mg/kg to about 14 mg/kg, about 10 mg/kg to about 12 mg/kg, about 12 mg/kg to about 40 mg/kg, about 12 mg/kg to about 38 mg/kg, about 12 mg/kg to about 36 mg/kg, about 12 mg/kg to about 34 mg/kg, about 12 mg/kg to about 32 mg/kg, about 12 mg/kg to about 30 mg/kg, about 12 mg/kg to about 28 mg/kg, about 12 mg/kg to about 26 mg/kg, about 12 mg/kg to about 24 mg/kg, about 12 mg/kg to about 22 mg/kg, about 12 mg/kg to about 20 mg/kg, about 12 mg/kg to about 18 mg/kg, about 12 mg/kg to about 16 mg/kg, about 12 mg/kg to about 14 mg/kg, about 14 mg/kg to about 40 mg/kg, about 14 mg/kg to about 38 mg/kg, about 14 mg/kg to about 36 mg/kg, about 14 mg/kg to about 34 mg/kg, about 14 mg/kg to about 32 mg/kg, about 14 mg/kg to about 30 mg/kg, about 14 mg/kg to about 28 mg/kg, about 14 mg/kg to about 26 mg/kg, about 14 mg/kg to about 24 mg/kg, about 14 mg/kg to about 22 mg/kg, about 14 mg/kg to about 20 mg/kg, about 14 mg/kg to about 18 mg/kg, about 14 mg/kg to about 16 mg/kg, about 16 mg/kg to about 40 mg/kg, about 16 mg/kg to about 38 mg/kg, about 16 mg/kg to about 36 mg/kg, about 16 mg/kg to about 34 mg/kg, about 16 mg/kg to about 32 mg/kg, about 16 mg/kg to about 30 mg/kg, about 16 mg/kg to about 28 mg/kg, about 16 mg/kg to about 26 mg/kg, about 16 mg/kg to about 24 mg/kg, about 16 mg/kg to about 22 mg/kg, about 16 mg/kg to about 20 mg/kg, about 16 mg/kg to about 18 mg/kg, about 18 mg/kg to about 40 mg/kg, about 18 mg/kg to about 38 mg/kg, about 18 mg/kg to about 36 mg/kg, about 18 mg/kg to about 34 mg/kg, about 18 mg/kg to about 32 mg/kg, about 18 mg/kg to about 30 mg/kg, about 18 mg/kg to about 28 mg/kg, about 18 mg/kg to about 26 mg/kg, about 18 mg/kg to about 24 mg/kg, about 18 mg/kg to about 22 mg/kg, about 18 mg/kg to about 20 mg/kg, about 20 mg/kg to about 40 mg/kg, about 20 mg/kg to about 38 mg/kg, about 20 mg/kg to about 36 mg/kg, about 20 mg/kg to about 34 mg/kg, about 20 mg/kg to about 32 mg/kg, about 20 mg/kg to about 30 mg/kg, about 20 mg/kg to about 28 mg/kg, about 20 mg/kg to about 26 mg/kg, about 20 mg/kg to about 24 mg/kg, about 20 mg/kg to about 22 mg/kg, about 22 mg/kg to about 40 mg/kg, about 22 mg/kg to about 38 mg/kg, about 22 mg/kg to about 36 mg/kg, about 22 mg/kg to about 34 mg/kg, about 22 mg/kg to about 32 mg/kg, about 22 mg/kg to about 30 mg/kg, about 22 mg/kg to about 28 mg/kg, about 22 mg/kg to about 26 mg/kg, about 22 mg/kg to about 24 mg/kg, about 24 mg/kg to about 40 mg/kg, about 24 mg/kg to about 38 mg/kg, about 24 mg/kg to about 36 mg/kg, about 24 mg/kg to about 34 mg/kg, about 24 mg/kg to about 32 mg/kg, about 24 mg/kg to about 30 mg/kg, about 24 mg/kg to about 28 mg/kg, about 24 mg/kg to about 26 mg/kg, about 26 mg/kg to about 40 mg/kg, about 26 mg/kg to about 38 mg/kg, about 26 mg/kg to about 36 mg/kg, about 26 mg/kg to about 34 mg/kg, about 26 mg/kg to about 32 mg/kg, about 26 mg/kg to about 30 mg/kg, about 26 mg/kg to about 28 mg/kg, about 28 mg/kg to about 40 mg/kg, about 28 mg/kg to about 38 mg/kg, about 28 mg/kg to about 36 mg/kg, about 28 mg/kg to about 34 mg/kg, about 28 mg/kg to about 32 mg/kg, about 28 mg/kg to about 30 mg/kg, about 30 mg/kg to about 40 mg/kg, about 30 mg/kg to about 38 mg/kg, about 30 mg/kg to about 36 mg/kg, about 30 mg/kg to about 34 mg/kg, about 30 mg/kg to about 32 mg/kg, about 32 mg/kg to about 40 mg/kg, about 32 mg/kg to about 38 mg/kg, about 32 mg/kg to about 36 mg/kg, about 32 mg/kg to about 34 mg/kg, about 34 mg/kg to about 40 mg/kg, about 34 mg/kg to about 38 mg/kg, about 34 mg/kg to about 36 mg/kg, about 36 mg/kg to about 40 mg/kg, about 36 mg/kg to about 38 mg/kg, about 38 mg/kg to about 40 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg at intervals of about 7 days (±2 days), about 14 days (±2 days), about 21 days (±2 days), or about 30 days (±2 days) during the period of time. In one embodiment, nivolumab or a biosimilar thereof is administered intravenously at a dose of about 3 mg/kg during the period of time. In one embodiment, nivolumab or a biosimilar thereof is administered intravenously as a flat dose of about 20 mg to about 500 mg (e.g., about 20 mg to about 480 mg, about 20 mg to about 460 mg, about 20 mg to about 440 mg, about 20 mg to about 420 mg, about 20 mg to about 400 mg, about 20 mg to about 380 mg, about 20 mg to about 360 mg, about 20 mg to about 340 mg, about 20 mg to about 320 mg, about 20 mg to about 300 mg, about 20 mg to about 280 mg, about 20 mg to about 260 mg, about 20 mg to about 240 mg, about 20 mg to about 220 mg, about 20 mg to about 200 mg, about 20 mg to about 180 mg, about 20 mg to about 160 mg, about 20 mg to about 140 mg, about 20 mg to about 120 mg, about 20 mg to about 100 mg, about 20 mg to about 80 mg, about 20 mg to about 60 mg, about 20 mg to about 40 mg, about 40 mg to about 500 mg, about 40 mg to about 480 mg, about 40 mg to about 460 mg, about 40 mg to about 440 mg, about 40 mg to about 420 mg, about 40 mg to about 400 mg, about 40 mg to about 380 mg, about 40 mg to about 360 mg, about 40 mg to about 340 mg, about 40 mg to about 320 mg, about 40 mg to about 300 mg, about 40 mg to about 280 mg, about 40 mg to about 260 mg, about 40 mg to about 240 mg, about 40 mg to about 220 mg, about 40 mg to about 200 mg, about 40 mg to about 180 mg, about 40 mg to about 160 mg, about 40 mg to about 140 mg, about 40 mg to about 120 mg, about 40 mg to about 100 mg, about 40 mg to about 80 mg, about 40 mg to about 60 mg, about 60 mg to about 500 mg, about 60 mg to about 480 mg, about 60 mg to about 460 mg, about 60 mg to about 440 mg, about 60 mg to about 420 mg, about 60 mg to about 400 mg, about 60 mg to about 380 mg, about 60 mg to about 360 mg, about 60 mg to about 340 mg, about 60 mg to about 320 mg, about 60 mg to about 300 mg, about 60 mg to about 280 mg, about 60 mg to about 260 mg, about 60 mg to about 240 mg, about 60 mg to about 220 mg, about 60 mg to about 200 mg, about 60 mg to about 180 mg, about 60 mg to about 160 mg, about 60 mg to about 140 mg, about 60 mg to about 120 mg, about 60 mg to about 100 mg, about 60 mg to about 80 mg, about 80 mg to about 500 mg, about 80 mg to about 480 mg, about 80 mg to about 460 mg, about 80 mg to about 440 mg, about 80 mg to about 420 mg, about 80 mg to about 400 mg, about 80 mg to about 380 mg, about 80 mg to about 360 mg, about 80 mg to about 340 mg, about 80 mg to about 320 mg, about 80 mg to about 300 mg, about 80 mg to about 280 mg, about 80 mg to about 260 mg, about 80 mg to about 240 mg, about 80 mg to about 220 mg, about 80 mg to about 200 mg, about 80 mg to about 180 mg, about 80 mg to about 160 mg, about 80 mg to about 140 mg, about 80 mg to about 120 mg, about 80 mg to about 100 mg, about 100 mg to about 500 mg, about 100 mg to about 480 mg, about 100 mg to about 460 mg, about 100 mg to about 440 mg, about 100 mg to about 420 mg, about 100 mg to about 400 mg, about 100 mg to about 380 mg, about 100 mg to about 360 mg, about 100 mg to about 340 mg, about 100 mg to about 320 mg, about 100 mg to about 300 mg, about 100 mg to about 280 mg, about 100 mg to about 260 mg, about 100 mg to about 240 mg, about 100 mg to about 220 mg, about 100 mg to about 200 mg, about 100 mg to about 180 mg, about 100 mg to about 160 mg, about 100 mg to about 140 mg, about 100 mg to about 120 mg, about 120 mg to about 500 mg, about 120 mg to about 480 mg, about 120 mg to about 460 mg, about 120 mg to about 440 mg, about 120 mg to about 420 mg, about 120 mg to about 400 mg, about 120 mg to about 380 mg, about 120 mg to about 360 mg, about 120 mg to about 340 mg, about 120 mg to about 320 mg, about 120 mg to about 300 mg, about 120 mg to about 280 mg, about 120 mg to about 260 mg, about 120 mg to about 240 mg, about 120 mg to about 220 mg, about 120 mg to about 200 mg, about 120 mg to about 180 mg, about 120 mg to about 160 mg, about 120 mg to about 140 mg, about 140 mg to about 500 mg, about 140 mg to about 480 mg, about 140 mg to about 460 mg, about 140 mg to about 440 mg, about 140 mg to about 420 mg, about 140 mg to about 400 mg, about 140 mg to about 380 mg, about 140 mg to about 360 mg, about 140 mg to about 340 mg, about 140 mg to about 320 mg, about 140 mg to about 300 mg, about 140 mg to about 280 mg, about 140 mg to about 260 mg, about 140 mg to about 240 mg, about 140 mg to about 220 mg, about 140 mg to about 200 mg, about 140 mg to about 180 mg, about 140 mg to about 160 mg, about 160 mg to about 500 mg, about 160 mg to about 480 mg, about 160 mg to about 460 mg, about 160 mg to about 440 mg, about 160 mg to about 420 mg, about 160 mg to about 400 mg, about 160 mg to about 380 mg, about 160 mg to about 360 mg, about 160 mg to about 340 mg, about 160 mg to about 320 mg, about 160 mg to about 300 mg, about 160 mg to about 280 mg, about 160 mg to about 260 mg, about 160 mg to about 240 mg, about 160 mg to about 220 mg, about 160 mg to about 200 mg, about 160 mg to about 180 mg, about 180 mg to about 500 mg, about 180 mg to about 480 mg, about 180 mg to about 460 mg, about 180 mg to about 440 mg, about 180 mg to about 420 mg, about 180 mg to about 400 mg, about 180 mg to about 380 mg, about 180 mg to about 360 mg, about 180 mg to about 340 mg, about 180 mg to about 320 mg, about 180 mg to about 300 mg, about 180 mg to about 280 mg, about 180 mg to about 260 mg, about 180 mg to about 240 mg, about 180 mg to about 220 mg, about 180 mg to about 200 mg, about 200 mg to about 500 mg, about 200 mg to about 480 mg, about 200 mg to about 460 mg, about 200 mg to about 440 mg, about 200 mg to about 420 mg, about 200 mg to about 400 mg, about 200 mg to about 380 mg, about 200 mg to about 360 mg, about 200 mg to about 340 mg, about 200 mg to about 320 mg, about 200 mg to about 300 mg, about 200 mg to about 280 mg, about 200 mg to about 260 mg, about 200 mg to about 240 mg, about 200 mg to about 220 mg, about 220 mg to about 500 mg, about 220 mg to about 480 mg, about 220 mg to about 460 mg, about 220 mg to about 440 mg, about 220 mg to about 420 mg, about 220 mg to about 400 mg, about 220 mg to about 380 mg, about 220 mg to about 360 mg, about 220 mg to about 340 mg, about 220 mg to about 320 mg, about 220 mg to about 300 mg, about 220 mg to about 280 mg, about 220 mg to about 260 mg, about 220 mg to about 240 mg, about 240 mg to about 500 mg, about 240 mg to about 480 mg, about 240 mg to about 460 mg, about 240 mg to about 440 mg, about 240 mg to about 420 mg, about 240 mg to about 400 mg, about 240 mg to about 380 mg, about 240 mg to about 360 mg, about 240 mg to about 340 mg, about 240 mg to about 320 mg, about 240 mg to about 300 mg, about 240 mg to about 280 mg, about 240 mg to about 260 mg, about 260 mg to about 500 mg, about 260 mg to about 480 mg, about 260 mg to about 460 mg, about 260 mg to about 440 mg, about 260 mg to about 420 mg, about 260 mg to about 400 mg, about 260 mg to about 380 mg, about 260 mg to about 360 mg, about 260 mg to about 340 mg, about 260 mg to about 320 mg, about 260 mg to about 300 mg, about 260 mg to about 280 mg, about 280 mg to about 500 mg, about 280 mg to about 480 mg, about 280 mg to about 460 mg, about 280 mg to about 440 mg, about 280 mg to about 420 mg, about 280 mg to about 400 mg, about 280 mg to about 380 mg, about 280 mg to about 360 mg, about 280 mg to about 340 mg, about 280 mg to about 320 mg, about 280 mg to about 300 mg, about 300 mg to about 500 mg, about 300 mg to about 480 mg, about 300 mg to about 460 mg, about 300 mg to about 440 mg, about 300 mg to about 420 mg, about 300 mg to about 400 mg, about 300 mg to about 380 mg, about 300 mg to about 360 mg, about 300 mg to about 340 mg, about 300 mg to about 320 mg, about 320 mg to about 500 mg, about 320 mg to about 480 mg, about 320 mg to about 460 mg, about 320 mg to about 440 mg, about 320 mg to about 420 mg, about 320 mg to about 400 mg, about 320 mg to about 380 mg, about 320 mg to about 360 mg, about 320 mg to about 340 mg, about 340 mg to about 500 mg, about 340 mg to about 480 mg, about 340 mg to about 460 mg, about 340 mg to about 440 mg, about 340 mg to about 420 mg, about 340 mg to about 400 mg, about 340 mg to about 380 mg, about 340 mg to about 360 mg, about 360 mg to about 500 mg, about 360 mg to about 480 mg, about 360 mg to about 460 mg, about 360 mg to about 440 mg, about 360 mg to about 420 mg, about 360 mg to about 400 mg, about 360 mg to about 380 mg, about 380 mg to about 500 mg, about 380 mg to about 480 mg, about 380 mg to about 460 mg, about 380 mg to about 440 mg, about 380 mg to about 420 mg, about 380 mg to about 400 mg, about 400 mg to about 500 mg, about 400 mg to about 480 mg, about 400 mg to about 460 mg, about 400 mg to about 440 mg, about 400 mg to about 420 mg, about 420 mg to about 500 mg, about 420 mg to about 480 mg, about 420 mg to about 460 mg, about 420 mg to about 440 mg, about 440 mg to about 500 mg, about 440 mg to about 480 mg, about 440 mg to about 460 mg, about 460 mg to about 500 mg, about 460 mg to about 480 mg, about 480 mg to about 500 mg, about 80 mg, about 150 mg, about 160 mg, about 200 mg, about 240 mg, about 250 mg, or about 300 mg), at intervals of about 7 days (±2 days), about 14 days (±2 days), about 21 days (±2 days), or about 30 days (±2 days) during the period of time. In one embodiment, nivolumab or a biosimilar thereof is administered intravenously as a flat dose of about 240 mg, during the period of time. In one embodiment, nivolumab or a biosimilar thereof is administered intravenously as a flat dose of about 480 mg, during the period of time. In one embodiment, nivolumab or a biosimilar thereof is administered intravenously over 60 minutes every two weeks, during the period of time. In one embodiment, nivolumab or a biosimilar thereof is administered intravenously over 60 minutes every four weeks, during the period of time. In one embodiment, nivolumab or a biosimilar thereof is administered intravenously over 30 minutes every four weeks, during the period of time. In one embodiment, nivolumab or a biosimilar thereof is administered intravenously as a flat dose of about 480 mg, wherein the nivolumab or a biosimilar thereof is administered intravenously over 30 minutes every four weeks, during the period of time.

In some embodiments, the PD-1 binding antagonist is pembrolizumab or a biosimilar thereof. In one embodiment, pembrolizumab or a biosimilar thereof is administered intravenously at a dose of about 1 mg/mg to about 40 mg/mg (e.g., or any of the subranges of this range described herein, e.g., about 1, 2, 3, 4, 5, 6, 7, 8, 9, 10, 11, 12, 13, 14, 15, 16, 17, 18, 19 or 20 mg/kg) at intervals of about 7 days (±2 days), about 14 days (±2 days), about 21 days (±2 days), or about 30 days (±2 days) during the period of time. In one embodiment, pembrolizumab or a biosimilar thereof is administered intravenously at a dose of about 2 mg/kg during the period of time. In one embodiment, pembrolizumab or a biosimilar thereof is administered intravenously as a flat dose of about 20 mg to about 500 mg (e.g., or any of the subranges of this range described herein, e.g., about 80, 150, 160, 200, 240, 250, or 300 mg) at intervals of about 7 days (±2 days), about 14 days (±2 days), about 21 days (±2 days), or about 30 days (±2 days) during the period of time. In one embodiment, pembrolizumab or a biosimilar thereof is administered intravenously as a flat dose of about 200 mg during the period of time. In one embodiment, pembrolizumab or a biosimilar thereof is administered intravenously every three weeks during the period of time.

In one embodiment, the invention provides a method for treating cancer comprising or consisting essentially of administering to a patient in need thereof, during a period of time, a combination therapy comprising, consisting essentially of, or consisting of therapeutically effective amounts, independently or in combination, of a CSF-1R inhibitor and a PD-1 binding antagonist, wherein the CSF-1R inhibitor is ARRY-382 or a pharmaceutically acceptable salt thereof. In one embodiment, the CSF-1R inhibitor is ARRY-382 as the free base. In one embodiment, ARRY-382 is orally administered once daily (QD) in the amount of about 200 mg or about 300 mg or about 400 mg during the period of time. In one embodiment, the PD-1 binding antagonist is nivolumab. In one embodiment, the PD-1 binding antagonist is pembrolizumab. In one embodiment, the amounts of the CSF-1R inhibitor and the PD-1 binding antagonist together achieve a synergistic effect in the treatment of cancer. In one embodiment, 200 mg of ARRY-382 is orally administered once daily during the period of time. In one embodiment, 300 mg of ARRY-382 is orally administered once daily during the period of time. In one embodiment, 400 mg of ARRY-382 is orally administered once daily during the period of time. In one embodiment, the patient, before the period of time, was previously administered one or more therapeutic agents that do not include or consist of a combination of a PD-1 binding antagonist and ARRY-382 or a pharmaceutically acceptable salt thereof.

In one embodiment, a method for treating cancer comprises or consists essentially of administering to a patient in need thereof, during a period of time, a combination therapy comprising, consisting essentially of, or consisting of therapeutically effective amounts, independently or in combination, of (a) a CSF-1R inhibitor which is ARRY-382 or a pharmaceutically acceptable salt thereof, and (b) a PD-1 binding antagonist which is nivolumab or a biosimilar thereof, wherein nivolumab or a biosimilar thereof is administered intravenously every two weeks during the period of time. In one embodiment, nivolumab or a biosimilar thereof is administered intravenously at a dose of about 3 mg/kg during the period of time. In one embodiment, nivolumab or a biosimilar thereof is administered intravenously as a flat dose of about 240 mg during the period of time. In one embodiment, the amounts of ARRY-382 and nivolumab or a biosimilar thereof together achieve a synergistic effect in the treatment of cancer (e.g., during the period of time). In one embodiment, the patient, before the period of time, was previously administered one or more therapeutic agents that do not include or consist of a combination of a PD-1 binding antagonist and ARRY-382 or a pharmaceutically acceptable salt thereof.

In one embodiment, a method for treating cancer comprises or consists essentially of administering to a patient in need thereof, during a period of time, a combination therapy comprising, consisting essentially of, or consisting of therapeutically effective amounts, independently or in combination, of (a) a CSF-1R inhibitor which is ARRY-382 or a pharmaceutically acceptable salt thereof, and (b) a PD-1 binding antagonist which is pembrolizumab or a biosimilar thereof, wherein pembrolizumab or a biosimilar thereof is administered intravenously every three weeks during the period of time. In one embodiment, pembrolizumab or a biosimilar thereof is administered intravenously at a dose of about 2 mg/kg, during the period of time. In one embodiment, pembrolizumab or a biosimilar thereof is administered intravenously as a flat dose of about 200 mg, during the period of time. In one embodiment, the amounts of ARRY-382 and nivolumab or a biosimilar thereof together achieve a synergistic effect in the treatment of cancer (e.g., during the period of time). In one embodiment, before the period of time, the patient was previously administered one or more therapeutic agents that do not include a combination of a PD-1 binding antagonist and ARRY-382 or a pharmaceutically acceptable salt thereof.

In one embodiment, a method for treating cancer comprises or consists essentially of administering to a patient in need thereof, during a period of time a combination therapy comprising, consisting essentially of, or consisting of therapeutically effective amounts, independently or in combination, of (a) a CSF-1R inhibitor which is ARRY-382 or a pharmaceutically acceptable salt thereof, wherein ARRY-382 is orally administered once daily in the amount of about 200 mg or about 300 mg or about 400 mg during the period of time, and (b) a PD-1 binding antagonist which is nivolumab or a biosimilar thereof, wherein nivolumab or a biosimilar thereof is administered intravenously every two weeks at a dose of about 3 mg/kg or as a flat dose of about 240 mg during the period of time. In one embodiment, the amounts of ARRY-382 and nivolumab or a biosimilar thereof together achieve a synergistic effect in the treatment of cancer (e.g., during the period of time). In one embodiment, 200 mg of ARRY-382 is orally administered once daily, during the period of time. In one embodiment, 300 mg of ARRY-382 is orally administered once daily, during the period of time. In one embodiment, 400 mg of ARRY-382 is orally administered once daily, during the period of time. In one embodiment, before the period of time, the subject was previously administered one or more therapeutic agents that do not include or consist of a combination of a PD-1 binding antagonist and ARRY-382 or a pharmaceutically acceptable salt thereof.

In one embodiment, a method for treating cancer comprises or consists essentially of administering to a patient in need thereof, during a period of time, a combination therapy comprising, consisting essentially of, or consisting of therapeutically effective amounts, independently or in combination, of (a) a CSF-1R inhibitor which is ARRY-382 or a pharmaceutically acceptable salt thereof, wherein ARRY-382 is orally administered once daily in the amount of about 200 mg or about 300 mg or about 400 mg during the period of time, and (b) a PD-1 binding antagonist which is pembrolizumab or a biosimilar thereof, wherein pembrolizumab or a biosimilar thereof is administered intravenously every three weeks during the period of time. In one embodiment, pembrolizumab or a biosimilar thereof is administered intravenously at a dose of about 2 mg/kg, during the period of time. In one embodiment, pembrolizumab or a biosimilar thereof is administered intravenously as a flat dose of about 200 mg, during the period of time. In one embodiment, 200 mg of ARRY-382 is orally administered once daily, during the period of time. In one embodiment, 300 mg of ARRY-382 is orally administered once daily, during the period of time. In one embodiment, 400 mg of ARRY-382 is orally administered once daily, during the period of time. In one embodiment, the amounts of ARRY-382 and pembrolizumab or biosimilar thereof together achieve a synergistic effect in the treatment of cancer (e.g., during the period of time). In one embodiment, before the period of time, the patient was previously administered one or more therapeutic agents that do not include or consist of a combination of a PD-1 binding antagonist and ARRY-382 or a pharmaceutically acceptable salt thereof.

In an embodiment, the invention is related to a method for treating cancer comprising or consisting essentially of administering to a patient in need thereof, during a period of time, a combination therapy comprising, consisting essentially of, or consisting of an amount of a CSF-1R inhibitor which is ARRY-382 or a pharmaceutically acceptable salt thereof and an amount of a PD-1 binding antagonist that is effective in treating cancer. In another embodiment, the invention is related to combination method comprising or consisting essentially of administering a CSF-1R inhibitor which is ARRY-382 or a pharmaceutically acceptable salt thereof, and a PD-1 binding antagonist, to a patient in need thereof, for a period of time. In another embodiment, the invention is related to a method for treating cancer comprising or consisting essentially of administering to a patient in need thereof, during a period of time, a combination therapy comprising, consisting essentially of, or consisting of an amount of a CSF-1R inhibitor which is ARRY-382 or a pharmaceutically acceptable salt thereof, and an amount of a PD-1 binding antagonist, wherein the amounts together achieve synergistic effects in the treatment of cancer. In another embodiment, the invention is related to a combination therapy method comprising or consisting essentially of administering, during a period of time, a CSF-1R inhibitor which is ARRY-382 or a pharmaceutically acceptable salt thereof, and a PD-1 binding antagonist, to a patient in need thereof, wherein the amounts of CSF-1R and ARRY-382 administered to the patient result in a synergistic effect (e.g., during the period of time). In one embodiment, the method or use of the invention is related to a synergistic combination therapy method comprising or consisting essentially of administering, during a period of time, a CSF-1R inhibitor which is ARRY-382 or a pharmaceutically acceptable salt thereof, in combination with a PD-1 binding antagonist, to a patient in need thereof. In one aspect of all the embodiments of this paragraph, the PD-1 binding antagonist is nivolumab or a biosimilar thereof. In one aspect of all the embodiments of this paragraph, the PD-1 binding antagonist is pembrolizumab or a biosimilar thereof.

Those skilled in the art will be able to determine, according to known methods, the appropriate amount, dose or dosage of each compound, as used in the combination of the present invention, to administer to a patient, taking into account factors such as age, weight, general health, the compound administered, the route of administration, the nature and advancement of the cancer requiring treatment, and the presence of other medications.

The practice of the method of this invention may be accomplished through various administration or dosing regimens. The compounds of the combination of the present invention can be administered concurrently, sequentially, or intermittently, and in any order.

Repetition of the administration or dosing regimens may be conducted as necessary to achieve the desired effect. A “continuous dosing schedule”, as used herein, is an administration or dosing regimen without dose interruptions, e.g., without days off treatment. Repetition of 21 or 28 day treatment cycles without dose interruptions between the treatment cycles is an example of a continuous dosing schedule. In an embodiment, one or both components of the combination of the present invention can be administered in a continuous dosing schedule.

In one embodiment, of any of the dosing regimens of a combination therapy as described herein, on days when the PD-1 binding antagonist is administered during the period of time, the PD-1 binding antagonist is administered at least 5 minutes, or at least 10 minutes, or at least 15 minutes, or at least 20 minutes, or at least 25 minutes, or at least 30 minutes, or at least 35 minutes, or at least 40 minutes, or at least 45 minutes, or at least 50 minutes, or at least 55 minutes, or at least 60 minutes, or at least 85 minutes, or at least 90 minutes after the administration of a therapeutically effective amount of the CSF-1R inhibitor which is ARRY-382 or a pharmaceutically acceptable salt thereof, during the period of time.

In one embodiment of any of the dosing regimens of a combination therapy as described herein, on days when the PD-1 binding antagonist is administered during the period of time, the PD-1 binding antagonist is administered 5 minutes, or at least 10 minutes, or at least 15 minutes, or at least 20 minutes, or at least 25 minutes, or at least 30 minutes, or at least 35 minutes, or at least 40 minutes, or at least 45 minutes, or at least 50 minutes, or at least 55 minutes, or at least 60 minutes, or at least 85 minutes, or at least 90 minutes before the administration of a therapeutically effective amount of the first therapeutically effective dose of the CSF-1R inhibitor which is ARRY-382 or a pharmaceutically acceptable salt thereof, during the period of time.

In one embodiment, the dose of the CSF-1R inhibitor which is ARRY-382 is escalated until the Maximum Tolerated Dosage is reached during the period of time, and the PD-1 binding antagonist is administered as a fixed dose during the period of time. Alternatively, the CSF-1R inhibitor which is ARRY-382 may be administered as a fixed dose during the period of time, and the dose of the PD-1 binding antagonist may be escalated until the Maximum Tolerated Dosage is reached during the period of time.

In some embodiments, a patient is administered one or more agents to ameliorate side effects of treatment during the period of time (e.g., one or more of corticosteroids, serotonin antagonists, dopamine antagonists, NK-1 inhibitors, cannabinoids, anti-anxiety drugs (e.g., lorazepam or diazepam), antibiotics, anti-fungal agents, colony-stimulating factor, iron supplements, Procrit, epoetin alfa, and darbepoetin alfa) during the period of time. In one embodiment, the one or more agents to ameliorate side effects of treatment is administered no sooner than 1 hour after administration of the CSF-1R inhibitor which is ARRY-382, during the period of time. In one embodiment, the one or more agents to ameliorate side effects of treatment is administered 30-60 minutes prior to the administration of the PD-1 binding antagonist, during the period of time. In one embodiment, the one or more agents to ameliorate side effects of treatment is administered 30 minutes prior administration of the PD-1 binding antagonist, during the period of time. In one embodiment, the one or more agents to ameliorate side effects of treatment is selected from one or more of a H1 antagonist (e.g., antihistamines such as diphenhydramine) and acetaminophen.

In one embodiment, before the period of time, the patient has previously been administered one or more therapeutic agents, e.g., chemotherapy. In one embodiment, before the period of time, the patient has previously been administered one or more therapeutic agents, e.g., a platinum-based chemotherapy. In one embodiment, before the period of time, the patient has previously been administered one or more therapeutic agents, e.g., a fluoropyrimidine-containing chemotherapy. In one embodiment, before the period of time, the patient has previously been administered one or more therapeutic agents, e.g., FOLFIRINOX or FOLFOXIRI (a chemotherapy regimen of folinic acid (leucovorin), fluorouracil (5-FU), irinotecan, and oxaliplatin). In one embodiment, before the period of time, the patient has previously been administered one or more therapeutic agents, e.g., irinotecan, oxaliplatin, and 5-fluorouracil). In one embodiment, the cancer has progressed after treatment with a platinum-based chemotherapy.

An improvement in a cancer or cancer-related disease can be characterized as a complete or partial response. “Complete response” or “CR” refers to an absence of clinically detectable disease with normalization of any previously abnormal radiographic studies, bone marrow, and cerebrospinal fluid (CSF) or abnormal monoclonal protein measurements. “Partial response” refers to at least about a 10%, 20%, 30%, 40%, 50%, 60%, 70%, 80%, or 90% decrease in all measurable tumor burden (i.e., the number of malignant cells present in the subject, or the measured bulk of tumor masses or the quantity of abnormal monoclonal protein) in the absence of new lesions.

Treatment may be assessed with one or more clinical endpoints, for example by inhibition of disease progression, inhibition of tumor growth, reduction of primary tumor, relief of tumor-related symptoms, inhibition of tumor secreted factors (including expression levels of checkpoint proteins as identified herein), delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), improved Time to tumor response (TTR), increased duration of response (DR), increased Progression Free Survival (PFS), increased Overall Survival (OS), Objective Response Rate (ORR), among others. OS as used herein means the time from treatment onset until death from any cause. TTP as used herein means the time from treatment onset until tumor progression; TTP does not comprise deaths. As used herein, TTR is defined for patients with confirmed objective response (CR or PR) as the time from the date of randomization or date of first dose of study treatment to the first documentation of objective tumor response. As used herein, DR means the time from documentation of tumor response to disease progression. As used herein, PFS means the time from treatment onset until tumor progression or death. As used herein, ORR means the proportion of patients with tumor size reduction of a predefined amount and for a minimum time period, where response duration usually is measured from the time of initial response until documented tumor progression. In the extreme, complete inhibition, is referred to herein as prevention or chemoprevention.

Thus, provided herein are methods for achieving one or more clinical endpoints associated with treating a cancer with a combination therapy described herein. In one embodiment, a patient described herein can show a positive tumor response, such as inhibition of tumor growth or a reduction in tumor size after treatment with a combination described herein. In certain embodiments, a patient described herein can achieve a Response Evaluation Criteria in Solid Tumors (for example, RECIST 1.1) of complete response, partial response or stable disease after administration of an effective amount a combination therapy described herein. In certain embodiments, a patient described herein can show increased survival without tumor progression. In some embodiments, a patient described herein can show inhibition of disease progression, inhibition of tumor growth, reduction of primary tumor, relief of tumor-related symptoms, inhibition of tumor secreted factors (including tumor secreted hormones, such as those that contribute to carcinoid syndrome), delayed appearance of primary or secondary tumors, slowed development of primary or secondary tumors, decreased occurrence of primary or secondary tumors, slowed or decreased severity of secondary effects of disease, arrested tumor growth and regression of tumors, decreased Time to Tumor Response (TTR), increased Duration of Response (DR), increased Progression Free Survival (PFS), increased Time To Progression (TTP), and/or increased Overall Survival (OS), among others.

In another embodiment, methods are provided for decreasing Time to Tumor Response (TTR), increasing Duration of Response (DR), increasing Progression Free Survival (PFS) of a patient having a cancer described herein, comprising administering an effective amount of a combination therapy as described herein. In one embodiment, a method is provided for decreasing Time to Tumor Response (TTR) of a patient having a cancer described herein, comprising administering an effective amount of a combination therapy as described herein. In one embodiment, is a method for increasing Progression Free Survival (PFS) of a patient a cancer described herein, comprising administering an effective amount of a combination therapy as described herein. In one embodiment, is a method for increasing Progression Free Survival (PFS) of a patient having a cancer described herein, comprising administering an effective amount of a combination therapy as described herein.

In one embodiment, the methods of treating cancer according to the invention also include surgery or radiotherapy. Non-limiting examples of surgery include, e.g., open surgery or minimally invasive surgery. Surgery can include, e.g., removing an entire tumor, debulking of a tumor, or removing a tumor that is causing pain or pressure in the subject. Methods for performing open surgery and minimally invasive surgery on a subject having a cancer are known in the art. Non-limiting examples of radiation therapy include external radiation beam therapy (e.g., external beam therapy using kilovoltage X-rays or megavoltage X-rays) or internal radiation therapy. Internal radiation therapy (also called brachytherapy) can include the use of, e.g., low-dose internal radiation therapy or high-dose internal radiation therapy. Low-dose internal radiation therapy includes, e.g., inserting small radioactive pellets (also called seeds) into or proximal to a cancer tissue in the subject. High-dose internal radiation therapy includes, e.g., inserting a thin tube (e.g., a catheter) or an implant into or proximal to a cancer tissue in the subject, and delivering a high dose of radiation to the thin tube or implant using a radiation machine. Methods for performing radiation therapy on a subject having a cancer are known in the art.

It may be shown by established test models that a combination therapy described herein results in the beneficial effects described herein before. The person skilled in the art is fully enabled to select a relevant test model to prove such beneficial effects. The pharmacological activity of a combination therapy described herein may, for example, be demonstrated in an animal model and/or a clinical study or in a test procedure, for example as described below.

Suitable clinical studies are, for example, open label, dose escalation studies in patients with a proliferative disease. Such studies may demonstrate in particular the synergism of the therapeutic agents of a combination therapy described herein. The beneficial effects on proliferative diseases may be determined directly through the results of these studies. Such studies may, in particular, be suitable for comparing the effects of a monotherapy using the CSF-1R inhibitor which is ARRY-382 and/or the PD-1 binding antagonist versus the effects of a combination therapy comprising the CSF-1R inhibitor which is ARRY-382 and the PD-1 binding antagonist.

The efficacy of the treatment may be determined in such studies, e.g., after 6, 12, 18 or 24 weeks by evaluation of symptom scores, e.g., every 6 weeks.

In some embodiments of any of the methods described herein, the patient is identified as having a tumor or a cancer cell that has increased level of PD-L1 and/or PD-L2 protein, e.g., as compared to a non-cancerous cell. Methods for determining a level of PD-L1 and PD-L2 in a tumor (e.g., a biopsy sample) or cancer cell are known in the art. Such methods include, e.g., immunoblotting, protein array, mass spectrometry, immunofluorescence microscopy, and fluorescence-assisted cell sorting (FACS). Additional methods for determining a level of PD-L1 and PD-L2 in a tumor (e.g., a biopsy sample) or a cancer cell are known in the art. Some embodiments of any of the methods described herein further include identifying a patient as having a tumor or a cancer cell that has an increased level of PD-L1 and/or PD-L2, and selecting the identified patient for treatment using any of the methods described herein. Some embodiments of any of the methods described herein can further include a step of selecting a subject identified as having a tumor or a cancer cell that has an increased level of PD-L1 and/or PD-L2, and the treating the patient using any of the methods described herein.

In some embodiments of any of the methods described herein, the patient is identified as having a tumor or a cancer cell that has increased level of CSF-1R and/or CSF-1 protein, or an increased level of CSF-1R activity, e.g., as compared to a non-cancerous cell. Methods for determining a level of CSF-1R and CSR-1, and the level of CSF-1R activity in a tumor (e.g., a biopsy sample) or cancer cell are known in the art. Such methods include, e.g., immunoblotting, protein array, mass spectrometry, immunofluorescence microscopy, fluorescence-assisted cell sorting (FACS), and RT-PCR. Additional methods for determining a level of CSF-1R and CSF-1, and the level of CSF-1R activity in a tumor (e.g., a biopsy sample) or a cancer cell are known in the art. Some embodiments of any of the methods described herein further include identifying a patient as having a tumor or a cancer cell that has an increased level of CSF-1R and/or CSF-1, and/or an increased level of CSF-1R activity, and selecting the identified patient for treatment using any of the methods described herein. Some embodiments of any of the methods described herein can further include a step of selecting a subject identified as having a tumor or a cancer cell that has an increased level of CSF-1R and/or CSF-1, and/or increased CSF-1R activity, and the treating the patient using any of the methods described herein.

In some embodiments of any of the methods described herein, the cancer is breast cancer, ovarian cancer, endometrial cancer, cervical cancer, acute myeloid leukemia, chronic myelocytic leukemia, myelodysplasia, hepatocellular cancer, idiopathic myelofibrosis, myelomonoblastic leukemia, pigmented villonodular synovitis, tenosynovial giant cell tumors, multiple myeloma, lung cancer, prostate cancer, gastric cancer, bladder cancer, Kaposi's sarcoma, or ovarian cancer.

In some embodiments of any of the methods described herein, the cancer is lung cancer, non-small cell lung (NSCL) cancer, bronchioloalveolar cell lung cancer, bone cancer, pancreatic cancer, skin cancer, cancer of the head or neck, cutaneous or intraocular melanoma, unresectable or metastatic melanoma, non-small cell lung cancer (NSCLC), metastatic NSCLC, head and neck squamous cell cancer, classical Hodgkin lymphoma, urothelial carcinoma, microsatellite instability-high cancer, hepatocellular cancer, microsatellite instability—high/mismatch repair deficient colorectal cancer, head and neck cancer, renal cell carcinoma, non-squamous non-small cell lung cancer, squamous non-small cell lung cancer, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, or lymphocytic leukemia, including refractory versions of any of the above cancers.

In some embodiments, the cancer is selected from the group consisting of: renal cancer, lung cancer, head and neck cancer, colon cancer, pancreatic cancer, breast cancer, prostate cancer, lung cancer, brain cancer, ovarian cancer, cervical cancer, testicular cancer, renal cancer, lymphoma, leukemia, melanoma, non-small cell lung cancer (NSCLC), colon cancer, colon carcinoma, colorectal carcinoma, skin cancer, metastatic melanoma, breast cancer, liver cancer, hepatoma, stomach cancer, head and neck cancer, bladder cancer, haematological cancer, lymphoma, and Hodgkin's lymphoma, osteosarcoma, neuroblastoma, glioma, glioblastoma multiforme, epitheloid carcinoma, esophageal cancer, and rectal cancer.

The compounds of the method or combination of the present invention may be formulated prior to administration. The formulation will preferably be adapted to the particular mode of administration. These compounds may be formulated with pharmaceutically acceptable carriers as known in the art and administered in a wide variety of dosage forms as known in the art. In making the pharmaceutical compositions of the present invention, the active ingredient will usually be mixed with a pharmaceutically acceptable carrier, or diluted by a carrier or enclosed within a carrier. Such carriers include, but are not limited to, solid diluents or fillers, excipients, sterile aqueous media and various non-toxic organic solvents. Dosage unit forms or pharmaceutical compositions include tablets, capsules, such as gelatin capsules, pills, powders, granules, aqueous and nonaqueous oral solutions and suspensions, lozenges, troches, hard candies, sprays, creams, salves, suppositories, jellies, gels, pastes, lotions, ointments, injectable solutions, elixirs, syrups, and parenteral solutions packaged in containers adapted for subdivision into individual doses.

Parenteral formulations include pharmaceutically acceptable aqueous or nonaqueous solutions, dispersion, suspensions, emulsions, and sterile powders for the preparation thereof. Examples of carriers include water, ethanol, polyols (propylene glycol, polyethylene glycol), vegetable oils, and injectable organic esters such as ethyl oleate. Fluidity can be maintained by the use of a coating such as lecithin, a surfactant, or maintaining appropriate particle size. Exemplary parenteral administration forms include solutions or suspensions of the compounds of the invention in sterile aqueous solutions, for example, aqueous propylene glycol or dextrose solutions. Such dosage forms can be suitably buffered, if desired.

Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art.

In one embodiment, the CSF-1R inhibitor which is ARRY-382 or a pharmaceutically acceptable salt thereof is formulated for oral administration. In one embodiment, ARRY-382 is formulated as a tablet or capsule. In one embodiment, ARRY-382 is formulated as a tablet. In one embodiment, the tablet is a coated tablet. In one embodiment, the CSF-1R inhibitor is ARRY-382 as the free base. In one embodiment, the CSF-1R inhibitor is a pharmaceutically acceptable salt of ARRY-382. In one embodiment, a capsule formulation of ARRY-382 comprises 25 mg of ARRY-382. In one embodiment, a capsule formulation of ARRY-382 comprises 50 mg of ARRY-382. In one embodiment, a capsule formulation of ARRY-382 comprises 100 mg of ARRY-382. In one embodiment, a capsule formulation of ARRY-382 comprises 200 mg of ARRY-382.

The invention also relates to a kit comprising the therapeutic agents of the combination of the present invention and written instructions for administration of the therapeutic agents. In one embodiment, the written instructions elaborate and qualify the modes of administration of the therapeutic agents, for example, for simultaneous or sequential administration of the therapeutic agents of the present invention. In one embodiment, the written instructions elaborate and qualify the modes of administration of the therapeutic agents, for example, by specifying the days of administration for each of the therapeutic agents during a 28 day cycle.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

### EXAMPLES

### Example 1. A Phase 1b/2 Dose-Escalation Study of ARRY-382, an Oral Inhibitor of Colony-Stimulating Factor-1 Receptor, in Combination with Pembrolizumab for the Treatment of Patients with Advanced Solid Tumors

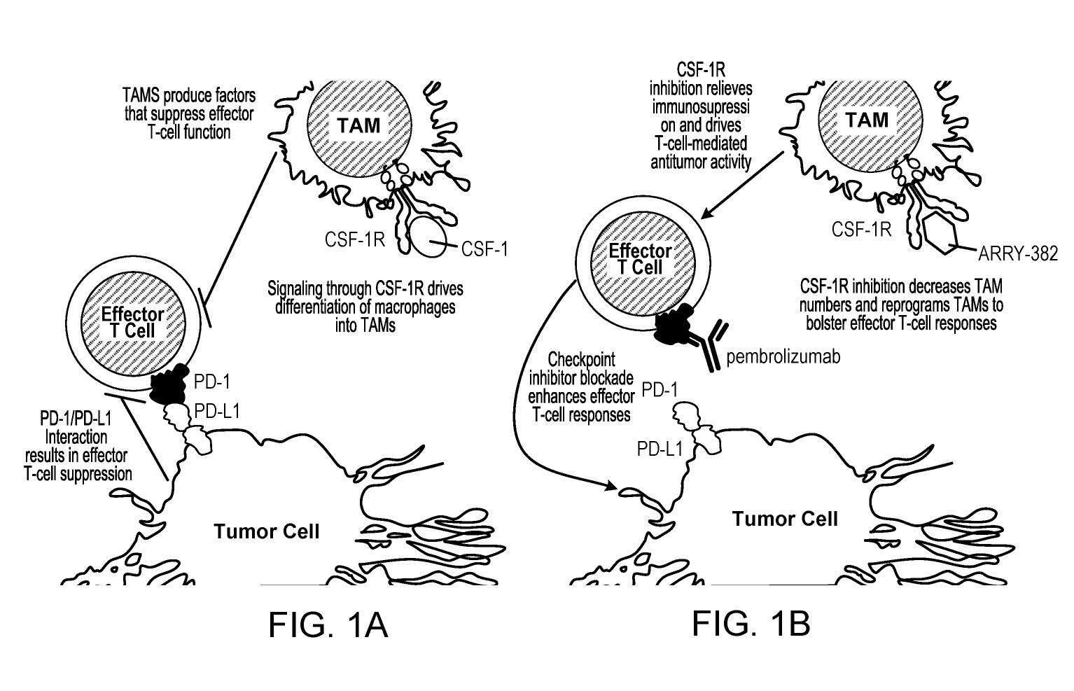

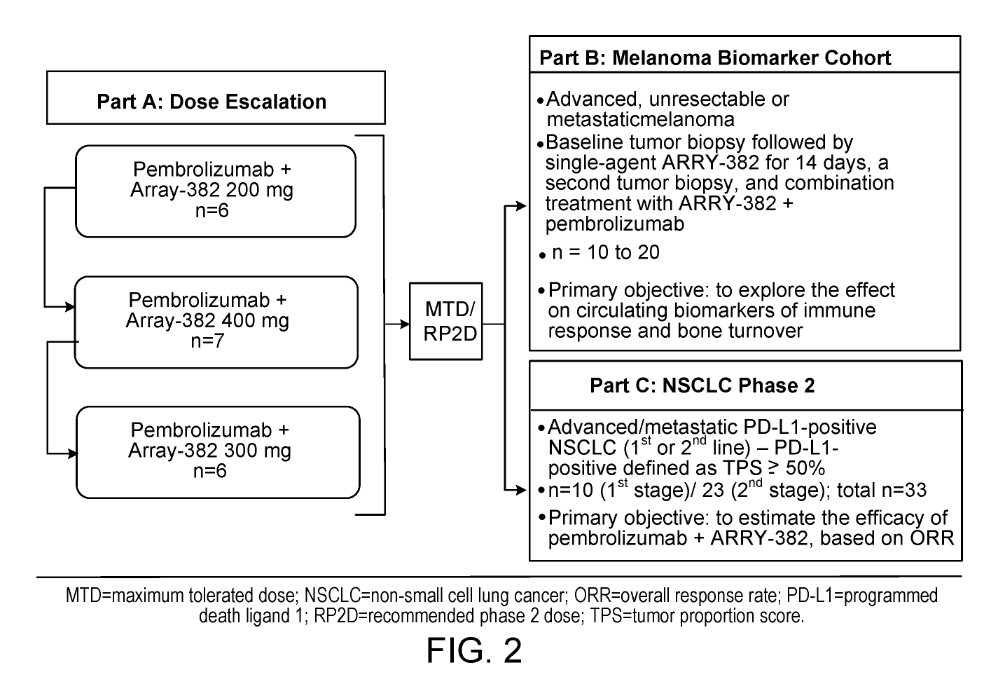

Tumor-associated macrophages (TAMs) and myeloid-derived suppressor cells are critical modulators of immune response in the tumor microenvironment (1). Colony-stimulating-factor-1 receptor (CSF-1R) signaling drives recruitment and differentiation of TAMs, which in turn enables tumor progression by suppressing antitumor immune responses, promoting angiogenesis, and facilitating tumor-cell metastasis (Hao et al., Clin Dev Immunol. 2012; 2012:948098, 3). Inhibition of CSF-1R signaling reduces the number of TAMs and alters their function, alleviating immunosuppression, enhancing antigen presentation, and inducing antitumor T-cell responses (4). CSF-1R inhibition also upregulates molecules associated with T-cell checkpoint signaling, which acts as a negative regulator of T-cell activity and cytokine production (Zhu et al., Cancer Res. 2014; 74(18):5057-5069; Postow et al., J Clin Oncol. 2015; 33(17):1974-1982). As shown in FIG. 1A, effector T-cell functions are inhibited by both TAMs and tumor cells. First, signaling through CSF-1R drives differentiation of macrophages into TAMs. Then, TAMs produce factors that suppress effector T-cell function. Tumor cells that express programmed death ligand (PD-L1) interact with programmed cell death protein 1 (PD-1) expressed on effector T-cells. This PD-1/PD-L1 interaction results in suppression of effector T-cell functions. CSF-1R inhibition also relieves immunosuppression and drives T-cell mediated antitumor activity.

The PD-1 blocking antibody pembrolizumab is approved for the treatment of multiple tumor types; however, the majority of patients do not respond to treatment (Sharma et al., Cell 2017; 168(4):707-723). ARRY-382 is a highly selective oral inhibitor of CSF-1R intracellular tyrosine kinase (Bendell et al., Mol Cancer Ther. 2013; 12(11)). The maximum tolerated dose (MTD) of ARRY-382 is 400 mg once daily (QD), with biological activity at doses greater than or equal to 200 mg QD (Bendell et al., Mol Cancer Ther. 2013; 12(11)).

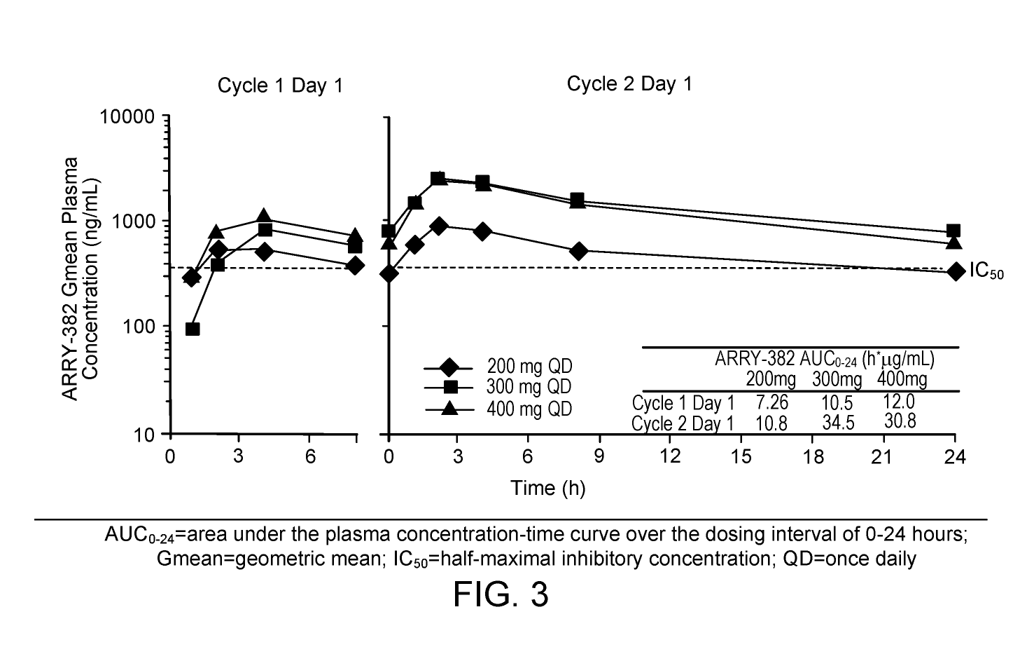

This study presents the initial findings from a phase 1b/2 study assessing ARRY-382 in combination with pembrolizumab in patients with advanced solid tumors. The objective of the phase 1b (Part A) portion of the study was to determine the MTD and/or recommended phase 2 dose (RP2D) of ARRY-382 in combination with intravenous pembrolizumab 2 mg/kg every 3 weeks, based on the incidence of dose-limiting toxicities (DLTs). This was a 3-part, open-label, multicenter study (NCT02880371; FIG. 2). Part A (phase 1b) included a dose-escalation component with 2 planned dose cohorts (intravenous pembrolizumab 2 mg/kg every 3 weeks with oral ARRY-382 400 mg QD) and the potential for a third planned cohort. A third cohort was enrolled with pembrolizumab 2 mg/kg every 3 weeks and an intermediate dose of ARRY-382 of 300 mg QD, based on the results from the first 2 dose cohorts.

Key eligibility criteria for Part A was adult patients with confirmed diagnosis of: bladder cancer, head and neck squamous cell cancer, ovarian cancer, triple-negative breast cancer, gastric cancer, metastatic colorectal cancer, or pancreatic ductal adenocarcinoma, refractory to standard of car, with no standard therapy available or for which the patient refused standard therapy; advanced, unresectable, or metastatic melanoma with or without prior treatment; or advanced/metastatic PD-L1-positive non-small cell lung cancer (NSCLC) meeting any of the following criteria: (1) tumors without EGFR or ALK genomic aberrations in patients who had received no prior systemic chemotherapy or had disease progression on or after platinum-containing chemotherapy, or (2) tumors with EGFR or ALK genomic aberrations in patients with disease progression on a US Food and Drug Administration-approved therapy for EGFR or ALK genomic tumor aberrations. Patients who received prior immune checkpoint inhibitor treatment were excluded from the study.

DLTs were determined using standard definitions. Adverse events (AEs) and serious AEs were collected to analyze the safety and tolerability of treatment. Blood samples for plasma pharmacokinetic (PK) analysis for ARRY-382 and its metabolites were drawn predose and at specified time points predose (trough) and postdose (Pollard J W, Nat Rev Immunol. 2009; 9(4):259-270; 2, 4, and 8 hours after administration of ARRY-382) on cycle 1 day 1 and cycle 2 day 1. Blood samples for pharmacodynamics analysis of circulating biomarkers, including CSF-1, were collected on days 1, 8, and 15 of cycle 1 and on day 1 of all subsequent cycles. Preliminary antitumor activity was analyzed using (1) the Response Evaluation Criteria in Solid Tumors (RECIST) version 1.1: investigator-determined overall response rate (ORR) method, and (2) the Immune-related response criteria (irRC): investigator-determined immune-related response rate (Wolchok et al., Clin Cancer Res. 2009; 15(23):7412-7420).

Overall, 19 patients were treated in Part A (Table 3). 52% of patients were male, 63% had an Eastern Cooperative Oncology Group Performance Status (ECOG PS) of 0. The remaining patients were ECOG PS 1. The most common tumor type was pancreatic ductal adenocarcinoma (6 patients; 32%). Patients had received a median of 2 (range 0-5) prior regimens of systemic treatment. 42% had received 3 or more prior regimens of systemic treatment.

Six patients were enrolled in the ARRY-382 200 mg cohort. No DTLs occurred, and the study proceeded with the ARRY-382 400 mg cohort. 2 of 7 patients in the ARRY-382 400 mg cohort experienced a DLT; therefore, an intermediate dose cohort (300 mg) was enrolled (n=6). Of the treated patients, 3 (16%) were still on treatment and 16 discontinued treatment as of the data cutoff of Sep. 11, 2017 (Table 4).

### Adverse Events

The most common all-grade adverse events were increased laboratory values (AST, CK, ALT, alkaline phosphatase (ALP), lipase, amylase), fatigue, pyrexia, and nausea (Table 5). The most common grade 3/4 events (>10%) were increased AST, increased CK, rash, lipase increased, increased ALP, increased ALT, and anemia (Table 5). All of these events were grade 3, with the exception of one grade 4 event of increased blood CK. There were 5 patients with 3 AST elevation: 3 of these patients had hepatic metastases at baseline; grade 3 elevation occurred once in 4 patients. In patient 1, grade 3 elevation resolved following interruption of ARRY-382 and recurred a second time; the second occurrence resolved with a drug reduction. AST and CK elevations resolved in all patients following interruption of ARRY-382. Dose reduction was required in 2 patients with AST elevation. Dose reduction was required in 3 patients with CK elevation.

AST and CK increases may also be caused by on-target effects of CSF-1R inhibition in macrophages and Kupffer cells (Radi et al. Am J Pathol. 2011; 179(1):240-247). Immune-related adverse events, as classified by the investigators, were identified in 5 patients (Table 6).

One patient in the ARRY-382 200 mg cohort with stage IIIA ovarian cancer discontinued treatment because of an AE of pneumonitis with onset in cycle 4 (approximately 3 months after starting therapy). The patient had not received previous radiation therapy and had no known lung metastasis. There were 3 on-study deaths (on treatment or within 30 days of last dose); cause of death was disease progression in all cases. One patient with stage IV metastatic colorectal cancer treated in the ARRY-382 300 mg cohort was discontinued in cycle 3 because of disease progression. One patient with stage IV gastric cancer treated in the ARRY-382 400 mg cohort completed 2 cycles of therapy and was discontinued for disease progression. One patient with stage IV pancreatic cancer treated in the ARRY-382 400 mg cohort completed 14 days of dosing and was discontinued because of changes in the patient's condition determined by the investigator to be due to disease progression. Dose reductions occurred in 3 patients in the ARRY-382 300 mg cohort. Two patients had a dose reduction from ARRY-382 300 mg to 200 mg in cycle 2 (one due to grade 3 pancreatitis and one due to grade 3 rash); one of these patients had a further dose reduction to ARRY-382 100 mg in cycle 6 due to increased CK. The third patient had a dose reduction in cycle 4 (due to grade 3 CK increase) and had a further dose reduction to 100 mg in cycle 8 due to a recurrence of increased CK.

### Pharmacokinetics/Pharmacodynamics

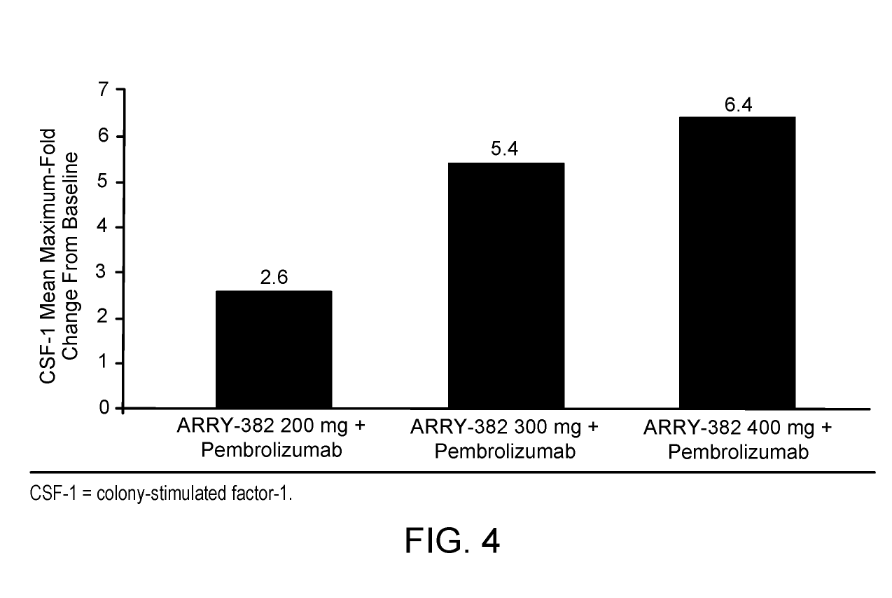

There was no evidence of any PK interactions between ARRY-382 and pembrolizumab. ARRY-382 exposures are generally dose-proportional (FIG. 3). Mean plasma concentration of ARRY-382 were >790 ng/mL over the full dosing interval following repeat dosing of >300 mg QD (cycle 2 day 1 minimum drug concentration (Cₘᵢₙ). The half-maximal inhibitory concentration (IC₅₀) of CSF-1R by ARRY-384 was 0.38 μg/mL determined using an ex vivo cell assay in which ARRY-383 inhibited CSF-1-mediated phosphorylation of extracellular signal-regulated kinase (ERK) in monocytes. The mean ARRY-382 Cₘᵢₙ was greater than the IC₅₀ with the 300 mg QD regimen, which suggested that continual target engagement was achievable (FIG. 3). Treatment with ARRY-382 300 mg QD resulted in an average maximum 5.4 fold increase in CSF-1 levels from baseline (FIG. 4).

### Efficacy

ORRs as measured via RECIST v1.1 and irRC are summarized in Table 7. Two patients achieved a partial response by both RECIST and irRC. One patient treated with ARRY-382 200 mg had stage III pancreatic ductal adenocarcinoma that had previously been treated with gemcitabine and paclitaxel in the neoadjuvant setting and FOLFOXIRI and FOLFOX in the adjuvant setting. The patient remained on study treatment in cycle 14. One patient treated with ARRY-382 300 mg had stage IV ovarian cancer with liver metastasis and had previously been treated with paclitaxel, gemcitabine, carboplatin, cisplatin, and doxorubicin in the neoadjuvant setting. The patient remained on study treatment in cycle 8. In the ARRY-382 300 mg cohort, stable disease was achieved by 2 patients (33%) per RECIST version 1.1 and 3 patients (50%) per irRC. A response to treatment was shown in FIG. 7.

The RP2D of ARRY-382 administered continuously on a daily basis was 300 mg QD in combination with pembrolizumab 2 mg/kg given intravenously every 3 weeks. ARRY-382 plus pembrolizumab had a manageable safety profile. The most common adverse events of any grade, regardless of relationship to study drug, were increased laboratory values (AST, ALT, CK, ALP, lipase, amylase), fatigue, pyrexia, and nausea. Grade 3 elevations of AST and CK were observed and were reversible following ARRY-382 dose interruption and, in some cases, dose reductions. Immune-related adverse events (regardless of relationship to study drug) were observed and were manageable. ARRY-382 PK was dose-proportional; at the 300 mg dose, exposure continually exceeded the IC₅₀ for CSF-1R inhibition in cell-based assays. Exposure was similar in the 300 mg and 400 mg dose cohorts. Increases from baseline in CSF-1 levels were correlated with increasing ARRY-382 exposure. The average maximum increase in CSF-1 levels from baseline was similar in the 300 mg and 400 mg cohorts. There were 2 partial responses, one each in the ARRY-382 200 mg and 300 mg cohort per both RECIST and irRC criteria; 2 and 3 patients (as assessed via RECIST version 1.1 and irRC, respectively) had stable disease.

### Example 2

### Study ARRAY-382-201

Clinical Study ARRAY-382-201 is a non-randomized, open-label, Phase 1b/2 study designed to determine the maximum tolerated dose (MTD) and/or recommended Phase 2 dose (RP2D) of ARRY-382 administered in combination with pembrolizumab in adult patients with select advanced solid tumors (Part A); to explore the effect of the combination on biomarkers of immune response in patients with select advanced solid tumors and to describe the preliminary antitumor activity of the combination in patients with select advanced solid tumors (Part B); and to estimate the efficacy of the combination in patients with programmed cell death ligand 1 (PD-L1)-positive (tumor proportion score [TPS]≥1%) non-small cell lung cancer (NSCLC; Part C) and in patients with microsatellite stable (MSS)/mismatch repair (MMR)-proficient pancreatic ductal adenocarcinoma (Part D).

### Part A

Part A (Phase 1b dose escalation) followed a standard dose-escalation design with 2 planned dose cohorts (ARRY-382 200 mg PO QD or 400 mg PO QD continuously plus pembrolizumab 2 mg/kg intravenously every 3 weeks). Patients in the 200 mg cohort were enrolled on a rolling basis with up to 6 evaluable patients or until dose-limiting toxicities (DLTs) were observed in more than 1 patient during Cycle 1. If fewer than 2 of 6 patients experienced a DLT during Cycle 1, patients in the next cohort were to receive ARRY-382 400 mg QD. If 2 or more patients in 200 mg cohort experienced DLTs during Cycle 1, the dosage of ARRY-382 in the next cohort was to be 100 mg QD. Intermediate doses and doses higher than 400 mg QD were also to be considered upon review of the safety and PK results from the planned dose cohorts.

### Key Eligibility Criteria for Part A:

Adult patients with confirmed diagnosis of the following disorders:
Bladder cancer, head and neck squamous cell cancer, ovarian cancer, triple-negative breast cancer, gastric cancer, metastatic colorectal cancer, or pancreatic ductal adenocarcinoma, refractory to standard of care, with no standard therapy available or for which the patient refused standard therapy
Advanced, unresectable, or metastatic melanoma with or without prior treatment
Advanced/metastatic PD-L1—positive NSCLC meeting any of the following criteria
   Tumors without EGFR or ALK genomic aberrations in patients who had received no prior systemic chemotherapy or had disease progression on or after platinum-containing chemotherapy
   Tumors with EGFR or ALK genomic aberrations in patients with disease progression on a US Food and Drug Administration-approved therapy for EGFR or ALK genomic tumor aberrations
      Patients who received prior immune checkpoint inhibitor treatment were excluded.

### Assessments for Part A

DLTs were determined using standard definitions.
Adverse events (AEs) and serious AEs were collected to analyze the safety and tolerability of treatment.
Blood samples for plasma pharmacokinetic (PK) analysis for ARRY-382 and its metabolites were drawn predose and at specified time points predose (trough) and postdose (1, 2, 4, and 8 hours after administration of ARRY-382) on cycle 1 day 1 and cycle 2 day 1.
Blood samples for pharmacodynamic analysis of circulating biomarkers, including CSF-1, were collected on days 1, 8, and 15 of cycle 1 and on day 1 of all subsequent cycles.
Preliminary antitumor activity was analyzed using the following methods:
   Response Evaluation Criteria in Solid Tumors (RECIST) version 1.1: investigator-determined overall response rate (ORR)
   Immune-related response criteria (irRC)9: investigator-determined immune-related response rate

Overall, 19 patients were treated in Part A (Table 3, above).

Six patients were enrolled in the ARRY-382 200-mg cohort. No DLTs occurred, and the study proceeded with the ARRY-382 400-mg cohort.

Two of seven patients in the ARRY-382 400-mg cohort experienced a DLT; therefore, an intermediate dose cohort (300 mg) was enrolled (n=6).

Of the treated patients, three (16%) were still on treatment and sixteen discontinued treatment as of the data cutoff of Sep. 11, 2017.

Pharmacokinetics/Pharmacodynamics

There was no evidence of any PK interactions between ARRY-382 and pembrolizumab.

ARRY-382 exposures were generally dose-proportional (FIG. 3).

Mean plasma concentrations of ARRY-382 were ≥790 ng/mL over the full dosing interval following repeat dosing of ≥300 mg QD (cycle 2 day 1 minimum drug concentration [Cmin]). The half-maximal inhibitory concentration (IC₅₀) of CSF-1R by ARRY-382 was 0.38 μg/mL determined using an ex vivo cell assay in which ARRY-382 inhibited CSF-1-mediated phosphorylation of extracellular signal-regulated kinase (ERK) in monocytes.

The mean ARRY-382 Cmin was greater than the IC₅₀ with the 300-mg QD regimen, which suggested that continual target engagement is achievable (FIG. 3).

Treatment with ARRY-382 300 mg QD resulted in an average maximum 5.4-fold increase in CSF-1 levels from baseline (FIG. 4).

The objective response rates (ORRs) as measured via RECIST (Response Evaluation Criteria in Solid Tumors) version 1.1 criteria and by irRC (immune-related response criteria) are summarized in Table 7 (above).

Two patients achieved a partial response by both RECIST and irRC. One patient treated with ARRY-382 200 mg had stage III pancreatic ductal adenocarcinoma that had previously been treated with gemcitabine and paclitaxel in the neoadjuvant setting and FOLFOXIRI and FOLFOX in the adjuvant setting. The patient remains on study treatment in cycle 14. One patient treated with ARRY-382 300 mg had stage IV ovarian cancer with liver metastasis and had previously been treated with paclitaxel, gemcitabine, carboplatin, cisplatin, and doxorubicin in the neoadjuvant setting. The patient remains on study treatment in cycle 8.

In the ARRY-382 300-mg cohort, stable disease was achieved by two patients (33%) per RECIST version 1.1 and 3 patients (50%) per irRC.

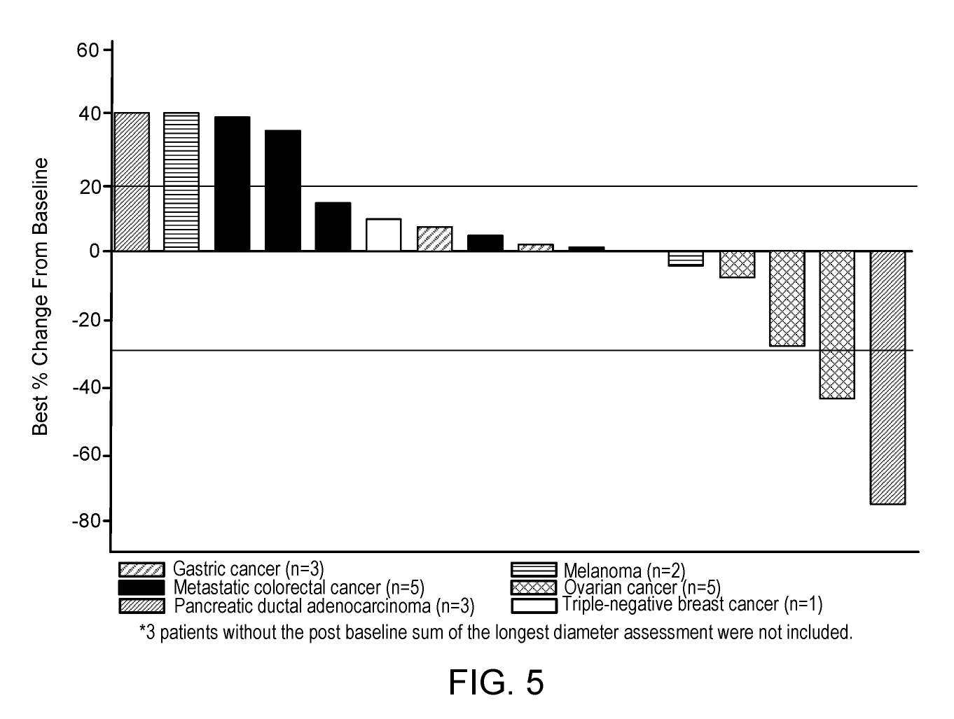

Best response to treatment is illustrated in FIG. 5.

### CONCLUSIONS

The RP2D of ARRY-382 administered continuously on a daily basis is 300 mg QD in combination with pembrolizumab 2 mg/kg given intravenously every 3 weeks.

ARRY-382 plus pembrolizumab has a manageable safety profile.

The most common AEs of any grade, regardless of relationship to study drug, were increased laboratory values (AST, ALT, CK, ALP, lipase, amylase), fatigue, pyrexia, and nausea.

Grade 3 elevations of AST and CK were observed and were reversible following ARRY-382 dose interruption and, in some cases, dose reductions.

Immune-related AEs (regardless of relationship to study drug) were observed and were manageable.

ARRY-382 PK was dose-proportional; at the 300-mg dose, exposure continually exceeded the IC₅₀ for CSF-1R inhibition in cell-based assays.

Exposure was similar in the 300-mg and 400-mg dose cohorts.

Increases from baseline in CSF-1 levels were correlated with increasing ARRY-382 exposure.

The average maximum increase in CSF-1 levels from baseline was similar in the 300-mg and 400-mg cohorts.

There were 2 partial responses, one each in the ARRY-382 200-mg and 300-mg cohort per both RECIST and irRC criteria; 2 and 3 patients (as assessed via RECIST version 1.1 and irRC, respectively) had stable disease.

ARRY-382 300-mg QD plus pembrolizumab 2 mg/kg every 3 weeks is currently being evaluated in Parts B and C of the study in patients with melanoma and NSCLC, respectively.

### Part B

Part B (Phase 1b expansion) consists of patients with select advanced solid tumors separated into 2 groups, B1 and B2, based on prior treatment. Patients with advanced unresectable/metastatic melanoma, urothelial cancer, head and neck squamous cell cancer (HNSCC), ovarian or colorectal cancer (CRC) in both Parts B1 and B2 plus patients with PD-L1-positive non-small cell lung cancer (NSCLC) in Part B2 are receiving single-agent ARRY-382 at the maximum tolerated dose/recommended phase 2 dose (MTD/RP2D) determined during Part A for 14 days (i.e., run-in period). Combination administration of ARRY-382 and pembrolizumab 200 mg IV Q3W will begin at the completion of the 14-day run-in period. Patients enrolled in Part B1 will be naïve to prior checkpoint inhibitors and must have ≥2 lesions that are accessible and amenable to biopsy. Patients enrolled in Part B2 must have been treated with a prior checkpoint inhibitor as their most recent prior therapy and have ≥1 lesion to be followed as a target lesion to assess efficacy or ≥2 lesions that are accessible and amenable to biopsy. Paired biopsies are mandatory in patients with ≥2 lesions that are accessible and amenable to biopsy. For patients with ≥2 lesions amenable to biopsy, metastatic lesions will be biopsied at Baseline and at the end of the 14-day run-in period.

Patients with PD-L1-positive NSCLC in Part C (Phase 2) and patients with MSS/MMR-proficient PDA in Part D (Phase 2) will receive ARRY-382 at the MTD/RP2D determined in Part A in combination with pembrolizumab 200 mg IV Q3W.

### Part C

ARRY-382 300-mg QD plus pembrolizumab 2 mg/kg every 3 weeks is currently being evaluated in Parts C of the study in patients with advanced/metastatic PD-L1-positive NSCLC (1^{st} or 2^{nd} line), where PD-L1 positive is defined as TPS≥50%. The primary objective is to estimate the efficacy of ARRY-382 plus pembrolizumab, based on ORR.

### Part D

Patients with MSS/MMR-proficient PDA in Part D (Phase 2) will receive ARRY-382 at the MTD/RP2D (300-mg QD) determined in Part A in combination with pembrolizumab 200 mg IV Q3W.

What is claimed:

1. A method for treating cancer comprising administering to a patient in need thereof, over a period of time, a combination therapy comprising an amount of a PD-1 binding antagonist and an amount of ARRY-382 or a pharmaceutically acceptable salt thereof, wherein the amounts together are effective in treating cancer.

2. The method of claim 1, wherein said cancer is selected from non-small cell lung cancer, pancreatic cancer, ovarian cancer, colorectal cancer, gastric cancer, melanoma, breast cancer, bladder cancer, head and neck cancer, uterine cancer, cervical cancer, liver cancer, thyroid cancer, kidney cancer, brain cancer, skin cancer, and mesothelioma.

3. The method of claim 2, wherein said cancer is non-small cell lung cancer.

4. The method of claim 3, wherein said non-small cell lung cancer is PD-L1 positive non-small cell lung cancer.

5. The method of claim 4, wherein said non-small cell lung cancer is advanced or metastatic PD-L1 positive non-small cell lung cancer.

6. The method of claim 5, wherein said cancer does not have an EGFR or ALK genomic aberration.

7. The method of claim 6, wherein said patient has not received chemotherapy prior to the period of time.

8. The method of claim 2, wherein said cancer is pancreatic cancer.

9. The method of claim 8, wherein said pancreatic cancer is pancreatic ductal adenocarcinoma.

10. The method of claim 9, wherein said pancreatic cancer is MSS/MMR-proficient pancreatic ductal adenocarcinoma.

11. The method of claim 2, wherein said cancer is ovarian cancer.

12. The method of claim 11, wherein said ovarian cancer is metastatic ovarian cancer.

13. The method of claim 2, wherein said cancer is melanoma.

14. The method of claim 13, wherein said melanoma is advanced, unresectable or metastatic melanoma.

15. The method of claim 2, wherein said cancer is breast cancer.

16. The method of claim 15, wherein said breast cancer is triple negative breast cancer.

17. The method of claim 2, wherein said cancer is bladder cancer.

18. The method of any one of claims 1 to 17, wherein the PD-1 binding antagonist is an anti PD-1 antibody.

19. The method of claim 18, wherein the anti PD-1 antibody is pembrolizumab or a biosimilar thereof.

20. The method of claim 19, wherein pembrolizumab is administered intravenously every three weeks over the period of time.

21. The method of claim 20 wherein pembrolizumab or the biosimilar thereof is administered intravenously at a dose of about 2 mg/kg during the period of time.

22. The method of claim 20, wherein pembrolizumab or the biosimilar thereof is administered intravenously as a flat dose of about 200 mg during the period of time.

23. The method of claim 18, wherein the anti PD-1 antibody is nivolumab or a biosimilar thereof.

24. The method of claim 23, wherein nivolumab or the biosimilar thereof is administered intravenously every two weeks during the period of time.

25. The method of claim 24, wherein nivolumab or the biosimilar thereof is administered intravenously every two weeks at a dose of about 3 mg/kg during the period of time.

26. The method of claim 24, wherein nivolumab or the biosimilar thereof is administered intravenously every two weeks as a flat dose of about 240 mg during the period of time.

27. The method of claim 23, wherein nivolumab or the biosimilar thereof is administered intravenously every four weeks during the period of time.

28. The method of claim 27, wherein nivolumab or the biosimilar thereof is administered intravenously every four weeks as a flat dose of about 480 mg during the period of time.

29. The method according to any one of claims 1-28, wherein ARRY-382 or a pharmaceutically acceptable salt thereof is administered orally once a day on a daily basis during the period of time.

30. The method according to claim 29, wherein ARRY-382 or a pharmaceutically acceptable salt thereof is administered orally in the amount of about 100 mg to about 300 mg once a day on a daily basis during the period of time.

31. The method according to claim 30, wherein ARRY-382 or a pharmaceutically acceptable salt thereof is administered orally in the amount of about 100 mg once a day on a daily basis during the period of time.

32. The method according to claim 30, wherein ARRY-382 or a pharmaceutically acceptable salt thereof is administered orally in the amount of about 200 mg once a day on a daily basis during the period of time.

33. The method according to claim 30, wherein ARRY-382 or a pharmaceutically acceptable salt thereof is administered orally in the amount of about 300 mg once a day on a daily basis during the period of time.

34. The method according to any one of claims 1-33, wherein the method further comprises assessing efficacy of treatment with said combination therapy, during the period of time, by determining one or more of inhibition of disease progression, inhibition of tumor growth, reduction of primary tumor, relief of tumor-related symptoms, inhibition of tumor secreted factors, delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS) or increased Duration of Response (DOR).

35. A method for treating cancer comprising administering to a patient in need thereof, during a period of time, a combination therapy comprising an amount of a PD-1 binding antagonist and an amount of a CSF-1R inhibitor which is ARRY-382 or a pharmaceutically acceptable salt thereof, wherein the amounts together are effective in treating cancer, wherein the patient was treated, prior to the period of time, with one or more therapeutic agent(s) that do not comprise a combination of a PD-1 binding antagonist and ARRY-382.

36. The method of claim 35, wherein the one or more therapeutic agent(s) is/are selected from one or more of a chemotherapy, a targeted anticancer agent, radiation therapy, and surgery.

37. The method of claim 36, wherein said chemotherapy is selected from one or more of a platinum-based chemotherapy and a fluoropyrimidine-containing therapy.

38. The method according to any one of claims 35-37, wherein said cancer is selected from non-small cell lung cancer, pancreatic cancer, ovarian cancer, colorectal cancer, gastric cancer, melanoma, breast cancer, bladder cancer, head and neck cancer, uterine cancer, cervical cancer, liver cancer, thyroid cancer, kidney cancer, brain cancer, skin cancer, and mesothelioma.

39. The method of claim 38, wherein said cancer is non-small cell lung cancer.

40. The method of claim 39, wherein said non-small cell lung cancer is PD-L1 positive non-small cell lung cancer.

41. The method of claim 40, wherein said non-small cell lung cancer is advanced or metastatic PD-L1 positive non-small cell lung cancer.

42. The method of claim 41, wherein said cancer has EGFR or ALK genomic aberration.

43. The method of claim 42, wherein said patient has received treatment with an EGFR inhibitor or an ALK inhibitor prior to treatment with said combination therapy.

44. The method of claim 38, wherein said cancer is pancreatic cancer.

45. The method of claim 44, wherein said cancer is pancreatic ductal adenocarcinoma.

46. The method of claim 45, wherein the one or more therapeutic agent(s) is/are selected from the group consisting of folinic acid, fluorouracil, oxaliplatin and irinotecan.

47. The method of claim 45, wherein the one or more therapeutic agents are folinic acid, fluorouracil, and oxaliplatin.

48. The method of claim 45, wherein the one or more therapeutic agents are gemcitabine and paclitaxel.

49. The method of claim 38, wherein said pancreatic cancer is MSS/MMR-proficient pancreatic ductal adenocarcinoma.

50. The method of claim 38, wherein said cancer is ovarian cancer

51. The method of claim 50, wherein said cancer is metastatic ovarian cancer.

52. The method of claim 51, wherein the one or more therapeutic agent(s) are selected from the group consisting of: paclitaxel, gemcitabine, carboplatin, cisplatin, and doxorubicin.

53. The method according to any one of claims 35-52, wherein the PD-1 binding antagonist is an anti PD-1 antibody.

54. The method according to claim 53, wherein the anti PD-1 antibody is pembrolizumab or a biosimilar thereof.

55. The method of claim 54, wherein pembrolizumab or the biosimilar thereof is administered intravenously every three weeks during the period of time.

56. The method of claim 55, wherein pembrolizumab or the biosimilar thereof is administered intravenously at a dose of about 2 mg/kg during the period of time.

57. The method of claim 55, wherein pembrolizumab or the biosimilar thereof is administered intravenously as a flat dose of about 200 mg during the period of time.

58. The method according to claim 53, wherein the anti PD-1 antibody is nivolumab or a biosimilar thereof.

59. The method of claim 58, wherein nivolumab or the biosimilar thereof is administered intravenously every two weeks during the period of time.

60. The method of claim 59, wherein nivolumab or the biosimilar thereof is administered intravenously every two weeks at a dose of about 3 mg/kg during the period of time.

61. The method of claim 59, wherein nivolumab or the biosimilar thereof is administered intravenously every two weeks as a flat dose of about 240 mg during the period of time.

62. The method of claim 58, wherein nivolumab or the biosimilar thereof is administered intravenously every four weeks during the period of time.

63. The method of claim 62, wherein nivolumab or the biosimilar thereof is administered intravenously every four weeks as a flat dose of about 480 mg during the period of time.

64. The method according to any one of claims 35-63, wherein ARRY-382 or a pharmaceutically acceptable salt thereof is administered orally once a day on a daily basis during the period of time.

65. The method according to claim 64, wherein ARRY-382 or a pharmaceutically acceptable salt thereof is administered orally in the amount of about 100 mg to about 300 mg once a day on a daily basis during the period of time.

66. The method according to claim 65, wherein ARRY-382 or a pharmaceutically acceptable salt thereof is administered orally in the amount of about 100 mg once a day on a daily basis during the period of time.

67. The method according to claim 65, wherein ARRY-382 or a pharmaceutically acceptable salt thereof is administered orally in the amount of about 200 mg once a day on a daily basis during the period of time.

68. The method according to claim 65, wherein ARRY-382 or a pharmaceutically acceptable salt thereof is administered orally in the amount of about 300 mg once a day on a daily basis during the period of time.

69. The method according to any one of claims 35-68, wherein the method further comprises assessing efficacy of treatment with said combination therapy, during the period of time, by determining one or more of inhibition of disease progression, inhibition of tumor growth, reduction of primary tumor, relief of tumor-related symptoms, inhibition of tumor secreted factors, delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS) or increased Duration of Response (DOR).

70. A combination therapy method comprising administering, over a period of time, to a patient in need thereof, therapeutically effective amounts, independently, of:
a CSF-1R inhibitor which is ARRY-382 or a pharmaceutically acceptable salt thereof; and
a PD-1 binding antagonist.

71. The combination therapy method according to claim 70, wherein the PD-1 binding antagonist is an anti PD-1 antibody.

72. The combination therapy method according to claim 71, wherein the anti PD-1 antibody is pembrolizumab or a biosimilar thereof.

73. The combination therapy method according to claim 71, wherein the anti PD-1 antibody is nivolumab or a biosimilar thereof.

74. The combination therapy method according to any one of claims 70-73, wherein said CSF-1R inhibitor is administered as a capsule.

75. The combination therapy method according to claim 74, wherein said capsule comprises about 25 mg, or about 50 mg, or about 75 mg, or about 100 mg of said CSF-1R inhibitor.

76. The combination therapy method according to claim 75, wherein said capsule comprises about 100 mg of said CSF-1R inhibitor.

77. The combination therapy method according to any one of claims 70-76, wherein said anti PD-1 antibody is administered by intravenous administration.

78. The combination therapy method according to any one of claims 70-77, wherein the patient has a cancer.

79. The combination therapy method according to claim 78, wherein said cancer is selected from non-small cell lung cancer, pancreatic cancer, ovarian cancer, colorectal cancer, gastric cancer, melanoma, breast cancer, bladder cancer head and neck cancer, uterine cancer, cervical cancer, liver cancer, thyroid cancer, kidney cancer, brain cancer, skin cancer, and mesothelioma.

80. The combination therapy method of claim 79, wherein said cancer is non-small cell lung cancer.

81. The combination therapy method of claim 80, wherein said non-small cell lung cancer is PD-L1 positive non-small cell lung cancer.

82. The combination therapy method of claim 81, wherein said non-small cell lung cancer is advanced or metastatic PD-L1 positive non-small cell lung cancer.

83. The combination therapy method of claim 82, wherein said cancer does not have an EGFR or ALK genomic aberration.

84. The combination therapy method of claim 82, wherein said cancer has an EGFR or ALK genomic aberration.

85. The combination therapy method of claim 79, wherein said cancer is pancreatic cancer.

86. The combination therapy method of claim 75, wherein said pancreatic cancer is pancreatic ductal adenocarcinoma.

87. The combination therapy method of claim 76, wherein said pancreatic cancer is MSS/MMR-proficient pancreatic ductal adenocarcinoma.

88. The combination therapy method of claim 79, wherein said cancer is ovarian cancer.

89. The combination therapy method of claim 88, wherein said ovarian cancer is metastatic ovarian cancer.

90. The combination therapy method of claim 79, wherein said cancer is melanoma.

91. The combination therapy method of claim 90, wherein said melanoma is advanced, unresectable or metastatic melanoma.

92. The combination therapy method of claim 80, wherein said cancer is breast cancer.

93. The combination therapy method of claim 92, wherein said breast cancer is triple negative breast cancer.

94. The combination therapy method of claim 79, wherein said cancer is bladder cancer.
